SEQUENCE LISTING (56 sequences)

SEQ ID NO: 1 - protein, 393 aa, Homo sapiens [sequence omitted]

SEQ ID NO: 2 - protein, 341 aa, Homo sapiens
MEEPQSDPSVEPPLSQETFSDLWKLLPENNVLSPLPSQAMDDLMLSPDDIEQWFTEDPGPDEAPRMPEAAPPVAPAPAAPTPAAPAPAPSWPLSSSVPSQKTYQGSYGFRLGFLHSGTAKSVTCTYSPALNKMFCQLAKTCPVQLWVDSTPPPGTRVRAMAIYKQSQHMTEVVRRCPHHERCSDSDGLAPPQHLIRVEGNLRVEYLDDRNTFRHSVVVPYEPPEVGSDCTTIHYNYMCNSSCMGGMNRRPILTIITLEDSSGNLLGRNSFEVRVCACPGRDRRTEEENLRKKGEPHHELPPGSTKRALPNNTSSSPQPKKKPLDGEYFTLQDQTSFQKENC

SEQ ID NO: 3 - protein, 346 aa, Homo sapiens
MEEPQSDPSVEPPLSQETFSDLWKLLPENNVLSPLPSQAMDDLMLSPDDIEQWFTEDPGPDEAPRMPEAAPPVAPAPAAPTPAAPAPAPSWPLSSSVPSQKTYQGSYGFRLGFLHSGTAKSVTCTYSPALNKMFCQLAKTCPVQLWVDSTPPPGTRVRAMAIYKQSQHMTEVVRRCPHHERCSDSDGLAPPQHLIRVEGNLRVEYLDDRNTFRHSVVVPYEPPEVGSDCTTIHYNYMCNSSCMGGMNRRPILTIITLEDSSGNLLGRNSFEVRVCACPGRDRRTEEENLRKKGEPHHELPPGSTKRALPNNTSSSPQPKKKPLDGEYFTLQMLLDLRWCYFLINSS

SEQ ID NO: 4 - protein, 354 aa, Homo sapiens
MDDLMLSPDDIEQWFTEDPGPDEAPRMPEAAPPVAPAPAAPTPAAPAPAPSWPLSSSVPSQKTYQGSYGFRLGFLHSGTAKSVTCTYSPALNKMFCQLAKTCPVQLWVDSTPPPGTRVRAMAIYKQSQHMTEVVRRCPHHERCSDSDGLAPPQHLIRVEGNLRVEYLDDRNTFRHSVVVPYEPPEVGSDCTTIHYNYMCNSSCMGGMNRRPILTIITLEDSSGNLLGRNSFEVRVCACPGRDRRTEEENLRKKGEPHHELPPGSTKRALPNNTSSSPQPKKKPLDGEYFTLQIRGRERFEMFRELNEALELKDAQAGKEPGGSRAHSSHLKSKKGQSTSRHKKLMFKTEGPDSD

SEQ ID NO: 5 - protein, 302 aa, Homo sapiens
MDDLMLSPDDIEQWFTEDPGPDEAPRMPEAAPPVAPAPAAPTPAAPAPAPSWPLSSSVPSQKTYQGSYGFRLGFLHSGTAKSVTCTYSPALNKMFCQLAKTCPVQLWVDSTPPPGTRVRAMAIYKQSQHMTEVVRRCPHHERCSDSDGLAPPQHLIRVEGNLRVEYLDDRNTFRHSVVVPYEPPEVGSDCTTIHYNYMCNSSCMGGMNRRPILTIITLEDSSGNLLGRNSFEVRVCACPGRDRRTEEENLRKKGEPHHELPPGSTKRALPNNTSSSPQPKKKPLDGEYFTLQDQTSFQKENC

SEQ ID NO: 6 - protein, 307 aa, Homo sapiens
MDDLMLSPDDIEQWFTEDPGPDEAPRMPEAAPPVAPAPAAPTPAAPAPAPSWPLSSSVPSQKTYQGSYGFRLGFLHSGTAKSVTCTYSPALNKMFCQLAKTCPVQLWVDSTPPPGTRVRAMAIYKQSQHMTEVVRRCPHHERCSDSDGLAPPQHLIRVEGNLRVEYLDDRNTFRHSVVVPYEPPEVGSDCTTIHYNYMCNSSCMGGMNRRPILTIITLEDSSGNLLGRNSFEVRVCACPGRDRRTEEENLRKKGEPHHELPPGSTKRALPNNTSSSPQPKKKPLDGEYFTLQMLLDLRWCYFLINSS

SEQ ID NO: 7 - protein, 261 aa, Homo sapiens
MFCQLAKTCPVQLWVDSTPPPGTRVRAMAIYKQSQHMTEVVRRCPHHERCSDSDGLAPPQHLIRVEGNLRVEYLDDRNTFRHSVVVPYEPPEVGSDCTTIHYNYMCNSSCMGGMNRRPILTIITLEDSSGNLLGRNSFEVRVCACPGRDRRTEEENLRKKGEPHHELPPGSTKRALPNNTSSSPQPKKKPLDGEYFTLQIRGRERFEMFRELNEALELKDAQAGKEPGGSRAHSSHLKSKKGQSTSRHKKLMFKTEGPDSD

SEQ ID NO: 8 - protein, 209 aa, Homo sapiens
MFCQLAKTCPVQLWVDSTPPPGTRVRAMAIYKQSQHMTEVVRRCPHHERCSDSDGLAPPQHLIRVEGNLRVEYLDDRNTFRHSVVVPYEPPEVGSDCTTIHYNYMCNSSCMGGMNRRPILTIITLEDSSGNLLGRNSFEVRVCACPGRDRRTEEENLRKKGEPHHELPPGSTKRALPNNTSSSPQPKKKPLDGEYFTLQDQTSFQKENC

SEQ ID NO: 9 - protein, 214 aa, Homo sapiens
MFCQLAKTCPVQLWVDSTPPPGTRVRAMAIYKQSQHMTEVVRRCPHHERCSDSDGLAPPQHLIRVEGNLRVEYLDDRNTFRHSVVVPYEPPEVGSDCTTIHYNYMCNSSCMGGMNRRPILTIITLEDSSGNLLGRNSFEVRVCACPGRDRRTEEENLRKKGEPHHELPPGSTKRALPNNTSSSPQPKKKPLDGEYFTLQMLLDLRWCYFLINSS

SEQ ID NO: 10 - protein, 393 aa, Homo sapiens [sequence omitted]

SEQ ID NO: 11 - DNA, 19231 bp, Artificial Sequence
  Description of Artificial Sequence Synthetic polynucleotide
ttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagctcgaaattaaccctcactaaagggaacaaaagctggtacgcggccgcaagtgataccagacacagccttcttattttttcttagaaccaacaatatatcggtccttggcccccacccccatgccccttttttccaatgctgggtgttgaacccggagccttagacataacacacgaactctgcagctgagctaccctgccagctccgaaagatttgtatactaagttctatgaaacattaaggtcgttttattcagggtaaggtgtggcttctggcttccctgggtccagtcctttgatggtggtgggtagtactgttcgttccattccgtttggggttttgattgacaagccttgcacctttccaactgttctacctcaagagccaaagataaagggtaaaagattcccttcccttatccctgtcaatagcagcctgcctagcttcctcaggatcaaatgagatgagcccctgagaagagcaaggcccgctgggcctggaaggccagccctggttgtactcaaacctctcgagtctattgcctttcccagccaacattttcttacacatccagcctctgtggatactgtgaccctcctgatctggttcttgtgaaaagtttcatattggcaactgttcttaaggaaacactgaaaacctaattactactaacgacctggaagatagagcaggaagacctcctattgtcagtgtggctatgtctccaagactgagacattgggccgccatcccagcatgtgcgcgcgcgcgcgcgcacacacacacacacacacacagtattcactaagcaagagttccttgacagggtgaactggcattaacgggtagtggttagcgccacagaagcactgctagctcagaacaaatgaggtgctcatacccacagggctgagctcttacatgagtgtgtactgttgggcgtggggtggggctggacccagggcttcacagatgagctccgcccaaggccaaatctcaaaaaatgaaaaggtcaaaaggtaaccctttcagatagataccggagatatgatatttacaaaaaatatgataattaaatgctacagttttaattatgaatctgaagcagctctgggtctccctgacccctttccagggccagccacctcattactcagatgtaggggctatataatacccagtcctttgctttcttcatctagctgattgcaagagaactgtgcctaagagcctgtgacgcactagattgtttgggtggccatctttaataaagagtcattcacctgcccaggggcagatgtgaccatcgagacagatgaaatagcccatatggaaaggtttctcaaactcttaaataaatgagaacttgattgttctacatacaagtgaatatattaaatgttgagattatagtcagtattaacagtaattattctgaaatgcttgcctttgctgttagaaaggaaacatgatgtcccagggggttggccgaactcaatccccagaacccacttcttataagttgtcatctgacctccatacgcatgtcttggcacctttaaataaggcacaataaataagtaaatgtctaagtaaggaagataatagctccgtggttagtaacaagagaaaatgagtgagagagtatcatacggcttaacctctgaaggaggtggtggtggtgtggctggctggagattggctggctgtgactgtctccgaggagctatggcatacaagataaggaagagcctacctcatagcctagggagaacagaaactgtgactttgctcttgtagaggtaaccacactgattcagccaggaggaagtaagtgctccctagagcccttggggaagaaggcagtaggaaacctgctgaatcttcaggaatttgtaaggcgctggggacctgtccctagggggcagatgagacactgatgggcgtacttagagatttgccatgaagtgggtttgaagaatggagctgtgtgtgaaatggtggatggggggggggagtccctccccagagggaagggaagagagatgagatgtagggtgcagatgtaggggggcttggggctagaagtacctccctgattacctgttccttgaagcagtgtgtggttcgagaagctgataggaagccaggccaaggcttgagcagcctgaataaaagacggaagagctgccccattcctgcttctctggaaatggtgtccctcacggccatcttgggtcctgacttcttctcaaaggagcctggccgacttcttggatacttgtaactttgcgctttcccaccctcgcataagtttcctgaaataatgactctgaaactcaaaatatatttacaaacacctcggctgtagctttggtttgttctctgacttgcttataacttaatatccctcttattctaacctaagttctgccacgtggttggttacctctgctcagcccccggcttctgtcctccatgttcctgggggaatccctacactttcagaatttaatttccctactggatgtcccaccttctttttattctaccctttcctataagccataggggtttgtttgtttgtatgttttttaattgacaagttatgcatccatacagtacacaatctcttctctctacagatgactgccatggaggagtcacagtcggatatcagcctcgagctccctctgagccaggagacattttcaggcttatggaaactgtgagtggatctttttggggcccttaagatacatcccgccatacctgtatcctccccttgcctgagagaaacaaaaacagtagtgttcaaacatggtatggtgttgggtgtctgtaaatcctgcggggcggggtggcggggggggggggggactgcagggtctcagaagtttgaggtcatcattgactacatagcaagttggaggccagcctgggataagtgagattctgtcttcaaaaaatggaaggaaatcaggaactaactctctgctcttgttttccagacttcctccagaagatatcctggtaaggcccagagcagaaagggacttgggctttggtgttgggctggtaggctgagaacacagtcctgagggttcttctttgtcccatccacagtcccccttgccgtcccaagcaatggatgatttgatgctgtccccggacgatattgaacaatggttcactgaagacccaggtccagatgaagctcccagaatgccagaggctgctccccccgtggcccctgcaccagcagctcctacaccggcggcccctgcaccagccccctcctggcccctgtcatcttctgtcccttcccagaaaacctaccagggcagctacggtttccgtctgggcttcttgcattctgggacagccaagtctgtgacttgcacggtcagttgccctgaggggctggcttccatgagacttcaatgcctggccgtatccccctgcatttcttttgtttggaactttgggattcctcttcaccctttggcttcctgtcagtgtttttttatagtttacccacttaatgtgtgatctctgactcctgtcccaaagttgaatattccccccttgaatttgggcttttatccatcccatcacaccctcagcatctctcctggggatgcagaacttttctttttcttcatccacgtgtattccttggcttttgaaaataagctcctgaccaggcttggtggctcacacctgcaatcccagcactctcaaagaggccaaggcaggcagatcacctgagcccaggagttcaagaccagcctgggtaacatgatgaaacctcgtctctacaaaaaaatacaaaaaattagccaggcatggtggtgcacacctatagtcccagccacttaggaggctgaggtgggaagatcacttgaggccaggagatggaggctgcagtgagctgtgatcacaccactgtgctccagcctgagtgacagagcaagaccctatctcaaaaaaaaaaaaaaaaaagaaaagctcctgaggtgtagacgccaactctctctagctcgctagtgggttgcaggaggtgcttacgcatgtttgtttctttgctgccgtcttccagttgctttatctgttcacttgtgccctgactttcaactctgtctccttcctcttcctacagtactcccctgccctcaacaagatgttttgccaactggccaagacctgccctgtgcagctgtgggttgattccacacccccgcccggcacccgcgtccgcgccatggccatctacaagcagtcacagcacatgacggaggttgtgaggcgctgcccccaccatgagcgctgctcagatagcgatggtgagcagctggggctggagagacgacagggctggttgcccagggtccccaggcctctgattcctcactgattgctcttaggtctggcccctcctcagcatcttatccgagtggaaggaaatttgcgtgtggagtatttggatgacagaaacacttttcgacatagtgtggtggtgccctgtgagccgcctgaggtctggtttgcaactggggtctctgggaggaggggttaagggtggttgtcagtggccctccaggtgagcagtaggggggctttctcctgctgcttatttgacctccctataaccccatgagatgtgcaaagtaaatgggtttaactattgcacagttgaaaaaactgaagcttacagaggctaagggcctcccctgcttggctgggcgcagtggctcatgcctgtaatcccagcactttgggaggccaaggcaggcggatcacgaggttgggagatcgagaccatcctggctaacggtgaaaccccgtctctactgaaaaatacaaaaaaaaattagccgggcgtggtgctgggcacctgtagtcccagctactcgggaggctgaggaaggagaatggcgtgaacctgggcggtggagcttgcagtgagctgagatcacgccactgcactccagcctgggcgacagagcgagattccatctcaaaaaaaaaaaaaaaaggcctcccctgcttgccacaggtctccccaaggcgcactggcctcatcttgggcctgtgttatctcctaggttggctctgactgtaccaccatccactacaactacatgtgtaacagttcctgcatgggcggcatgaaccggaggcccatcctcaccatcatcacactggaagactccaggtcaggagccacttgccaccctgcacactggcctgctgtgccccagcctctgcttgcctctgacccctgggcccacctcttaccgatttcttccatactactacccatccacctctcatcacatccccggcggggaatctccttactgctcccactcagttttcttttctctggctttgggacctcttaacctgtggcttctcctccacctacctggagctggagcttaggctccagaaaggacaagggtggttgggagtagatggagcctggttttttaaatgggacaggtaggacctgatttccttactgcctcttgcttctcttttcctatcctgagtagtggtaatctactgggacggaacagctttgaggtgcgtgtttgtgcctgtcctgggagagaccggcgcacagaggaagagaatctccgcaagaaaggggagcctcaccacgagctgcccccagggagcactaagcgaggtaagcaagcaggacaagaagcggtggaggagaccaagggtgcagttatgcctcagattcacttttatcacctttccttgcctctttcctagcactgcccaacaacaccagctcctctccccagccaaagaagaaaccactggatggagaatatttcacccttcaggtaccaaggctggagagtcgcatgccagagacaagctgtacccattattgcctctgtctctcgcatgtataaaatagtgttattagcaggttgccaggtctttttcagtggctttatctctagccgtgacattagcttgagagctcatgctctgaggctgtgcctcctccgacagtggttctcagtgtaactaacttgacaacaccaacttacaccataagacaggtgctcctccactggggatgggaacatgtcctaggaaactcaccataaattgaaaataacacacgacaaaaatgtgtttagaggcaggcctggtggcacctgcctgtaattacagcactggtcgacctgcagccaagctatcgaattcctgcagcccaattccgatcatattcaataacccttaatataacttcgtataatgtatgctatacgaagttattaggtctgaagaggagtttacgtccagccaagctagctccatgggccaggcaaatatcccttaccagcctcacagagacctcccccaccccccgcaaccctagagttcttttactagtgagggacaagtggacaatggtgctgttgtgggccccaccctgtgtcccctgtgcccacagtggtcactctgcttggcaggcaggtgttgcaggctggctgctccaggccctggcaggaggtactgaaggacctggtaggctcagatgccctggatgccaaggcactgctggagtacttccaaccggtcagccagtggctggaagagcagaatcagcggaatggcgaagtcctaggctggccagagaatcagtggcgtccaccgttacccgacaactatccagagggcattggtaaagctctgagtgagggtggactgggaccaagagaagtcctggcctctggcctctggcttctgggtcaaagcctcagcatcctggtcactttgctgccagctgagccccagtgtcctttgcttcagtgccaagccacccctgggctcatcctcagggccctaagcagaaatgggtatgtctttctctcagggtcctagagacagtgtgcccaagcctgagggcccttggggtcaggctggctggcacattgctctatgaggtcacactgcaggcttggctcttattggccggtgatgggagcttcagggctctgctttcctgcggccatgcccaagaagaagaggaaggtgtccaatttactgaccgtacaccaaaatttgcctgcattaccggtcgatgcaacgagtgatgaggttcgcaagaacctgatggacatgttcagggatcgccaggcgttttctgagcatacctggaaaatgcttctgtccgtttgccggtcgtgggcggcatggtgcaagttgaataaccggaaatggtttcccgcagaacctgaagatgttcgcgattatcttctatatcttcaggcgcgcggtctggcagtaaaaactatccagcaacatttgggccagctaaacatgcttcatcgtcggtccgggctgccacgaccaagtgacagcaatgctgtttcactggttatgcggcggatccgaaaagaaaacgttgatgccggtgaacgtgcaaaacaggctctagcgttcgaacgcactgatttcgaccaggttcgttcactcatggaaaatagcgatcgctgccaggatatacgtaatctggcatttctggggattgcttataacaccctgttacgtatagccgaaattgccaggatcagggttaaagatatctcacgtactgacggtgggagaatgttaatccatattggcagaacgaaaacgctggttagcaccgcaggtgtagagaaggcacttagcctgggggtaactaaactggtcgagcgatggatttccgtctctggtgtagctgatgatccgaataactacctgttttgccgggtcagaaaaaatggtgttgccgcgccatctgccaccagccagctatcaactcgcgccctggaagggatttttgaagcaactcatcgattgatttacggcgctaaggtaaatataaaatttttaagtgtataatgtgttaaactactgattctaattgtttgtgtattttaggatgactctggtcagagatacctggcctggtctggacacagtgcccgtgtcggagccgcgcgagatatggcccgcgctggagtttcaataccggagatcatgcaagctggtggctggaccaatgtaaatattgtcatgaactatatccgtaacctggatagtgaaacaggggcaatggtgcgcctgctggaagatggcgattagccattaacgcgtaaatgattgctataattatttgatatttatggtgacatatgagaaaggatttcaacatcgacggaaaatatgtagtgctgtctgtaagcactaatattcagtcgccagccgtcattgtcactgtaaagctgagcggcaataaaaagacagaataaaacgcacgggtgttgggtcgtttgttcggtcgagctcgcgaagctagcttggctgcaggtcgtcgaaattctaccgggtaggggaggcgcttttcccaaggcagtctggagcatgcgctttagcagccccgctgggcacttggcgctacacaagtggcctctggcctcgcacacattccacatccaccggtaggcgccaaccggctccgttctttggtggccccttcgcgccaccttctactcctcccctagtcaggaagttcccccccgccccgcagctcgcgtcgtgcaggacgtgacaaatggaagtagcacgtctcactagtctcgtgcagatggacagcaccgctgagcaatggaagcgggtaggcctttggggcagcggccaatagcagctttgctccttcgctttctgggctcagaggctgggaaggggtgggtccgggggcgggctcaggggcgggctcaggggcggggcgggcgcccgaaggtcctccggaggcccggcattctgcacgcttcaaaagcgcacgtctgccgcgctgttctcctcttcctcatctccgggcctttcgacctgcagcctgttgacaattaatcatcggcatagtatatcggcatagtataatacgacaaggtgaggaactaaaccatgggatcggccattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgatgatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgaggggatcaattctctagagctcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaaagaaccagctggggctcgactagagcttgcggaacccttaatataacttcgtataatgtatgctatacgaagttattaggtccctcgagaagagggtggttcaaagctggcaagaaaaagtaacccaaggaaaggacattggggtttaagggtataactcagttccaggatacttgccaagtgtgtgccaggccccggattaggtccccagcggctcacggtgcctttagtccacctaacccacaactggactctacagctcagctcccggttgccctgttaagcgtctgttccctctaactgaaaagatccaagcttgttgtacacgttctactgaatgcctgttactttcacaccatcttattaaagatgatctcccccccccccaaaaaaaaaaacaaaacaaaacaaaacaaaaacctgtaagtggagccagcttaagttgggaaccaactttcagaaagaaagttgttaaaatcgtgaaagtggttgtgtgaccttgtccagtgcttccatctcacttcatctctgctgcagatccgcgggcgtaaacgcttcgagatgttccgggagctgaatgaggccttagagttaaaggatgcccatgctacagaggagtctggagacagcagggctcactccaggtaagtggcctggggcagcgcctgcctgtggtgctctacccagacctccctccagctcagcctttgtagtgaaagataaaaaccccaccctgctagatgcttagggctgcaccctacgagaactgacttcttgactttttaggctctgttaaggggtatgagggacaaggtatggtgtcatgctcctataatctcagcagtaaggaagacaaagtcaggaggatttggggaagtttgaagccttcataaactatataaaactttaggccagctagggctagctacaatagcaagaccctgtctcatggtgatggtgatgatggtggtggtggtgacagttgtgataataatggcagtggtggtgatgatgatggtggtggtgatgatggtgatggtgaaagggaggataaactgattctcagaagtattccagtgtgttctgtgaatatccctacccatagtagaagccatcttaaattcctttttttcagcctccagcctagagccttccaagccttgatcaaggaggaaagcccaaactgctagctcccatcacttcatccctccccttttctgtcttcctatagctacctgaagaccaagaagggccagtctacttcccgccataaaaaaacaatggtcaagaaagtggggcctgactcagactgactgcctctgcatcccgtccccatcaccagcctccccctctccttgctgtcttatgacttcagggctgagacacaatcctcccggtcccttctgctgccttttttaccttgtagctagggctcagccccctctctgagtagtggttcctggcccaagttggggaataggttgatagttgtcaggtctctgctggcccagcgaaattctatccagccagttgttggaccctggcacctacaatgaaatctcaccctaccccacaccctgtaagattctatcttgggccctcatagggtccatatcctccagggcctactttccttccattctgcaaagcctgtctgcatttatccaccccccaccctgtctccctcttttttttttttttacccctttttatatatcaatttcctattttacaataaaattttgttatcacttatatggttttgagaggttgatatcagcataagctgtctgggcccccaggggcaggatgaatttgggaggtacccacctgagtccaggcagtctgttggagtcaggggtggggaacttgggttccagaagaggaacaaagccggggactgggtcagtctgtgggctgcatgacaacaagagagcagggtgactccattcataacttgggaaccaactgtccctcctccctcctgccaggactggcacatggtccttacccacccctacttctagggttgggctcctgctgtcctctggggagcctctcaccaaggattaagggatttaaatgtctgatatagcaaacctgagcctctggagtgaccatctgctccacaagaaaggatcagggtgcctgggttccacagggaagggggtggctgttcctggatgaagagacaagtgggaggcccgccagctggggtcccaggaaactgggagcagttaaggtgaggctaggggccttcccagcatccccaaactccgggcctcacgccaggcaagtgaatgaatcgaagctttgcactttgccaggaaatcctttgaaagggcttcttggagtagggaggagagtcagagttggggagcctaacaccctctcacccacccccttcctcagtgctgctggctcccagagagcgggactaggccaagctctccccatagcctgctgagccagtgccagtagggagagcagtcggtgagcacatggcttggccatggctcttggtaacagagagaggggtactgaggtccagaggcactttggggccactgtcctctgcctgtcccaggcttaaagagccagtgagagtcctttgccttgcccgagggtcctatacaagttgggagggagcagccccttagcctcaaaattttgtacttgtgagtaccaaatattttcgcagcagactagggtctatctttttaactgttgtgggaatgtgtgatgccagtcaaacccactacgcttttcccctgtttttgtcccacagctttactgagaactaaatccaaagaactgtgtttaagggccacaaatttgagcccttatctcaaaaagggaggaggggctaaaacaattgccaaatgaaaagctatctctgcatttgtgcctgtatggtaaacgttaagactgacagagaactactcaacaaaagctcctccacccaactcctttatttttttcagacagtaccttgctaactaagtagcccaggctaccctcaacattggtctctttatttagctttccaagtggagggagggatcgatgggctaccatacttccttaccttccccatttccctatcttttgtccttaattctgtccttgtacctcaagtcttcagaaagcaggtccattttggtacctcaaatcccttctctgagagctgatatcaatggacaaaggataatgaggtttttgcttctcctgcaagctcttctatgtaacttcacatcagtcacttacctcccaggttccacttccgggctgtttccattgatgcatcaactaggcatcgatccataaagtacttgacattcacagggaccctatgtctgctatgatgtctgcccaagaggtaaatacctcaggccttactctattgaggtctaaaatcaaacctcccaatctctaatcttgtaactctaacctacttttacattttaggtcaatatattttttattttttgtattttgagaaatggtggcgtgccagaccatgataagcaagacattgtaccagcaattaaccccccacccccaactccatagctgggtggtgatggagcacacctttaatcctggcacttgggacacagaggcaggcagagttcagcctagtaaacagagtgagttccagtccagccagggctatacagagaaaccctgtctcaaaaaaaacaaaacaaaaacaccaaacaaacaggaaaatcaaccccaggctggagagatggctcagcagttaagagcactgactgttcttccaaaggtcctgagttcaaatcccagcaagcacatggtggctcacaaccatctgacatctgatgccctctacaggtgtgtctgaagatagctacagtgtacttacatataaataataaatctttttttaaaaaaagaagaaaagaaaaatcaacccctattctggcttgatttttgtgatggtttcaagatcttgttgttgctgctgttttgttttgttaatttctgagatgatggagtcttgtacaacacaggctagctctaactcctgattctacctctacctccaaaatgctgagattataataaacaagtttcatgactcccagtacatatattgtttgtttggttggttggtttggtttgttttttttcgagacaaggttcctctgtatagccctggctgtcctggaactcactttgtagaccagtctggcctcgaactcagaaatctgcctgcctctgcctcccaagtgctgggaatacttttttaatttagaaattaatttaagaagcatgttgggtgccgggtgtggtggcgcactcctttaatcccagcactcgggaggcagaggcaggcggatttctgagttcgaggccagactggtctacaaagtgagttccaggacagccagggctacacagagaaaccctgtctcaaaaaaccaaaaaaaaaaaaaaaaaaaaaagcatgttggatctccaaatttgaggccagcctggtctacagcacaagttccaggacaaacacagacaaaccctatttcaaacatacatagggtcaggaggcaaaggcaggtagatctctgtgagtaccagtctcgcttgcctggtctatatagtgagttctaggtattcaggaaataacctagataatgttagccccaagtgctgtcattatttaagagagagagaaaataaaggaacctaggggctagagaagtggctcaatgattatgaaagagtacaagctgctcttccagaggaccagggttctgatcccagcacccatgtcaagtcactcacaaatccatatcaccaagagattcaccctcggcgcgccactcttcgcgacagctagatctcatcgcctaggatcgcccgggttgattcgaggctgctaacaaatcgagtcgagcatcgagcagtgtggttttcaagaggaagcaaaaagcctctccacccaggcctggaatgtttccacccaatgtcgagcagtgtggttttgcaagaggaagcaaaaagcctctccacccaggcctggaatgtttccacccaatgtcgagcaaaccccgcccagcgtcttgtcattggcgaattcgaacacgcagatgcagtcggggcggcgcggtcccaggtccacttcgcatattaaggtgacgcgtgtggcctcgaacaccgagcgaccctgcagcgacccgcttaacagcgtcaacagcgtgccgcagatcttggtggcgtgaaactcccgcacctcttcggccagcgccttgtagaagcgcgtgccatggatcctgatgatgttgttgattcttctaaatcttttgtgatggaaaacttttcttcgtaccacgggactaaacctggttatgtagattccattcaaaaaggtatacaaaagccaaaatctggtacacaaggaaattatgacgatgattggaaagggttttatagtaccgacaataaatacgacgctgcgggatactctgtagataatgaaaacccgctctctggaaaagctggaggcgtggtcaaagtgacgtatccaggactgacgaaggttctcgcactaaaagtggataatgccgaaactattaagaaagagttaggtttaagtctcactgaaccgttgatggagcaagtcggaacggaagagtttatcaaaaggttcggtgatggtgcttcgcgtgtagtgctcagccttcccttcgctgaggggagttctagcgttgaatatattaataactgggaacaggcgaaagcgttaagcgtagaacttgagattaattttgaaacccgtggaaaacgtggccaagatgcgatgtatgagtatatggctcaagcctgtgcaggaaatcgtgtcaggcgatctctttgtgaaggaaccttacttctgtggtgtgacataattggacaaactacctacagagatttaaagctctaaggtaaatataaaatttttaagtgtataatgtgttaaactactgattctaattgtttgtgtattttagattccaacctatggaactgatgaatgggagcagtggtggaatgcagatcctagagctcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaaagaaccagcccgggcggtggagctccaattcgccctatagtgagtcgtattacaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggaaattgtaagcg

SEQ ID NO: 12 - DNA, 19231 bp, Artificial Sequence
  Description of Artificial Sequence Synthetic polynucleotide
ttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagctcgaaattaaccctcactaaagggaacaaaagctggtacgcggccgcaagtgataccagacacagccttcttattttttcttagaaccaacaatatatcggtccttggcccccacccccatgccccttttttccaatgctgggtgttgaacccggagccttagacataacacacgaactctgcagctgagctaccctgccagctccgaaagatttgtatactaagttctatgaaacattaaggtcgttttattcagggtaaggtgtggcttctggcttccctgggtccagtcctttgatggtggtgggtagtactgttcgttccattccgtttggggttttgattgacaagccttgcacctttccaactgttctacctcaagagccaaagataaagggtaaaagattcccttcccttatccctgtcaatagcagcctgcctagcttcctcaggatcaaatgagatgagcccctgagaagagcaaggcccgctgggcctggaaggccagccctggttgtactcaaacctctcgagtctattgcctttcccagccaacattttcttacacatccagcctctgtggatactgtgaccctcctgatctggttcttgtgaaaagtttcatattggcaactgttcttaaggaaacactgaaaacctaattactactaacgacctggaagatagagcaggaagacctcctattgtcagtgtggctatgtctccaagactgagacattgggccgccatcccagcatgtgcgcgcgcgcgcgcgcacacacacacacacacacacagtattcactaagcaagagttccttgacagggtgaactggcattaacgggtagtggttagcgccacagaagcactgctagctcagaacaaatgaggtgctcatacccacagggctgagctcttacatgagtgtgtactgttgggcgtggggtggggctggacccagggcttcacagatgagctccgcccaaggccaaatctcaaaaaatgaaaaggtcaaaaggtaaccctttcagatagataccggagatatgatatttacaaaaaatatgataattaaatgctacagttttaattatgaatctgaagcagctctgggtctccctgacccctttccagggccagccacctcattactcagatgtaggggctatataatacccagtcctttgctttcttcatctagctgattgcaagagaactgtgcctaagagcctgtgacgcactagattgtttgggtggccatctttaataaagagtcattcacctgcccaggggcagatgtgaccatcgagacagatgaaatagcccatatggaaaggtttctcaaactcttaaataaatgagaacttgattgttctacatacaagtgaatatattaaatgttgagattatagtcagtattaacagtaattattctgaaatgcttgcctttgctgttagaaaggaaacatgatgtcccagggggttggccgaactcaatccccagaacccacttcttataagttgtcatctgacctccatacgcatgtcttggcacctttaaataaggcacaataaataagtaaatgtctaagtaaggaagataatagctccgtggttagtaacaagagaaaatgagtgagagagtatcatacggcttaacctctgaaggaggtggtggtggtgtggctggctggagattggctggctgtgactgtctccgaggagctatggcatacaagataaggaagagcctacctcatagcctagggagaacagaaactgtgactttgctcttgtagaggtaaccacactgattcagccaggaggaagtaagtgctccctagagcccttggggaagaaggcagtaggaaacctgctgaatcttcaggaatttgtaaggcgctggggacctgtccctagggggcagatgagacactgatgggcgtacttagagatttgccatgaagtgggtttgaagaatggagctgtgtgtgaaatggtggatggggggggggagtccctccccagagggaagggaagagagatgagatgtagggtgcagatgtaggggggcttggggctagaagtacctccctgattacctgttccttgaagcagtgtgtggttcgagaagctgataggaagccaggccaaggcttgagcagcctgaataaaagacggaagagctgccccattcctgcttctctggaaatggtgtccctcacggccatcttgggtcctgacttcttctcaaaggagcctggccgacttcttggatacttgtaactttgcgctttcccaccctcgcataagtttcctgaaataatgactctgaaactcaaaatatatttacaaacacctcggctgtagctttggtttgttctctgacttgcttataacttaatatccctcttattctaacctaagttctgccacgtggttggttacctctgctcagcccccggcttctgtcctccatgttcctgggggaatccctacactttcagaatttaatttccctactggatgtcccaccttctttttattctaccctttcctataagccataggggtttgtttgtttgtatgttttttaattgacaagttatgcatccatacagtacacaatctcttctctctacagatgactgccatggaggagtcacagtcggatatcagcctcgagctccctctgagccaggagacattttcaggcttatggaaactgtgagtggatctttttggggcccttaagatacatcccgccatacctgtatcctccccttgcctgagagaaacaaaaacagtagtgttcaaacatggtatggtgttgggtgtctgtaaatcctgcggggcggggtggcggggggggggggggactgcagggtctcagaagtttgaggtcatcattgactacatagcaagttggaggccagcctgggataagtgagattctgtcttcaaaaaatggaaggaaatcaggaactaactctctgctcttgttttccagacttcctccagaagatatcctggtaaggcccagagcagaaagggacttgggctttggtgttgggctggtaggctgagaacacagtcctgagggttcttctttgtcccatccacagtcccccttgccgtcccaagcaatggatgatttgatgctgtccccggacgatattgaacaatggttcactgaagacccaggtccagatgaagctcccagaatgccagaggctgctccccgcgtggcccctgcaccagcagctcctacaccggcggcccctgcaccagccccctcctggcccctgtcatcttctgtcccttcccagaaaacctaccagggcagctacggtttccgtctgggcttcttgcattctgggacagccaagtctgtgacttgcacggtcagttgccctgaggggctggcttccatgagacttcaatgcctggccgtatccccctgcatttcttttgtttggaactttgggattcctcttcaccctttggcttcctgtcagtgtttttttatagtttacccacttaatgtgtgatctctgactcctgtcccaaagttgaatattccccccttgaatttgggcttttatccatcccatcacaccctcagcatctctcctggggatgcagaacttttctttttcttcatccacgtgtattccttggcttttgaaaataagctcctgaccaggcttggtggctcacacctgcaatcccagcactctcaaagaggccaaggcaggcagatcacctgagcccaggagttcaagaccagcctgggtaacatgatgaaacctcgtctctacaaaaaaatacaaaaaattagccaggcatggtggtgcacacctatagtcccagccacttaggaggctgaggtgggaagatcacttgaggccaggagatggaggctgcagtgagctgtgatcacaccactgtgctccagcctgagtgacagagcaagaccctatctcaaaaaaaaaaaaaaaaaagaaaagctcctgaggtgtagacgccaactctctctagctcgctagtgggttgcaggaggtgcttacgcatgtttgtttctttgctgccgtcttccagttgctttatctgttcacttgtgccctgactttcaactctgtctccttcctcttcctacagtactcccctgccctcaacaagatgttttgccaactggccaagacctgccctgtgcagctgtgggttgattccacacccccgcccggcacccgcgtccgcgccatggccatctacaagcagtcacagcacatgacggaggttgtgaggcgctgcccccaccatgagcgctgctcagatagcgatggtgagcagctggggctggagagacgacagggctggttgcccagggtccccaggcctctgattcctcactgattgctcttaggtctggcccctcctcagcatcttatccgagtggaaggaaatttgcgtgtggagtatttggatgacagaaacacttttcgacatagtgtggtggtgccctgtgagccgcctgaggtctggtttgcaactggggtctctgggaggaggggttaagggtggttgtcagtggccctccaggtgagcagtaggggggctttctcctgctgcttatttgacctccctataaccccatgagatgtgcaaagtaaatgggtttaactattgcacagttgaaaaaactgaagcttacagaggctaagggcctcccctgcttggctgggcgcagtggctcatgcctgtaatcccagcactttgggaggccaaggcaggcggatcacgaggttgggagatcgagaccatcctggctaacggtgaaaccccgtctctactgaaaaatacaaaaaaaaattagccgggcgtggtgctgggcacctgtagtcccagctactcgggaggctgaggaaggagaatggcgtgaacctgggcggtggagcttgcagtgagctgagatcacgccactgcactccagcctgggcgacagagcgagattccatctcaaaaaaaaaaaaaaaaggcctcccctgcttgccacaggtctccccaaggcgcactggcctcatcttgggcctgtgttatctcctaggttggctctgactgtaccaccatccactacaactacatgtgtaacagttcctgcatgggcggcatgaaccggaggcccatcctcaccatcatcacactggaagactccaggtcaggagccacttgccaccctgcacactggcctgctgtgccccagcctctgcttgcctctgacccctgggcccacctcttaccgatttcttccatactactacccatccacctctcatcacatccccggcggggaatctccttactgctcccactcagttttcttttctctggctttgggacctcttaacctgtggcttctcctccacctacctggagctggagcttaggctccagaaaggacaagggtggttgggagtagatggagcctggttttttaaatgggacaggtaggacctgatttccttactgcctcttgcttctcttttcctatcctgagtagtggtaatctactgggacggaacagctttgaggtgcgtgtttgtgcctgtcctgggagagaccggcgcacagaggaagagaatctccgcaagaaaggggagcctcaccacgagctgcccccagggagcactaagcgaggtaagcaagcaggacaagaagcggtggaggagaccaagggtgcagttatgcctcagattcacttttatcacctttccttgcctctttcctagcactgcccaacaacaccagctcctctccccagccaaagaagaaaccactggatggagaatatttcacccttcaggtaccaaggctggagagtcgcatgccagagacaagctgtacccattattgcctctgtctctcgcatgtataaaatagtgttattagcaggttgccaggtctttttcagtggctttatctctagccgtgacattagcttgagagctcatgctctgaggctgtgcctcctccgacagtggttctcagtgtaactaacttgacaacaccaacttacaccataagacaggtgctcctccactggggatgggaacatgtcctaggaaactcaccataaattgaaaataacacacgacaaaaatgtgtttagaggcaggcctggtggcacctgcctgtaattacagcactggtcgacctgcagccaagctatcgaattcctgcagcccaattccgatcatattcaataacccttaatataacttcgtataatgtatgctatacgaagttattaggtctgaagaggagtttacgtccagccaagctagctccatgggccaggcaaatatcccttaccagcctcacagagacctcccccaccccccgcaaccctagagttcttttactagtgagggacaagtggacaatggtgctgttgtgggccccaccctgtgtcccctgtgcccacagtggtcactctgcttggcaggcaggtgttgcaggctggctgctccaggccctggcaggaggtactgaaggacctggtaggctcagatgccctggatgccaaggcactgctggagtacttccaaccggtcagccagtggctggaagagcagaatcagcggaatggcgaagtcctaggctggccagagaatcagtggcgtccaccgttacccgacaactatccagagggcattggtaaagctctgagtgagggtggactgggaccaagagaagtcctggcctctggcctctggcttctgggtcaaagcctcagcatcctggtcactttgctgccagctgagccccagtgtcctttgcttcagtgccaagccacccctgggctcatcctcagggccctaagcagaaatgggtatgtctttctctcagggtcctagagacagtgtgcccaagcctgagggcccttggggtcaggctggctggcacattgctctatgaggtcacactgcaggcttggctcttattggccggtgatgggagcttcagggctctgctttcctgcggccatgcccaagaagaagaggaaggtgtccaatttactgaccgtacaccaaaatttgcctgcattaccggtcgatgcaacgagtgatgaggttcgcaagaacctgatggacatgttcagggatcgccaggcgttttctgagcatacctggaaaatgcttctgtccgtttgccggtcgtgggcggcatggtgcaagttgaataaccggaaatggtttcccgcagaacctgaagatgttcgcgattatcttctatatcttcaggcgcgcggtctggcagtaaaaactatccagcaacatttgggccagctaaacatgcttcatcgtcggtccgggctgccacgaccaagtgacagcaatgctgtttcactggttatgcggcggatccgaaaagaaaacgttgatgccggtgaacgtgcaaaacaggctctagcgttcgaacgcactgatttcgaccaggttcgttcactcatggaaaatagcgatcgctgccaggatatacgtaatctggcatttctggggattgcttataacaccctgttacgtatagccgaaattgccaggatcagggttaaagatatctcacgtactgacggtgggagaatgttaatccatattggcagaacgaaaacgctggttagcaccgcaggtgtagagaaggcacttagcctgggggtaactaaactggtcgagcgatggatttccgtctctggtgtagctgatgatccgaataactacctgttttgccgggtcagaaaaaatggtgttgccgcgccatctgccaccagccagctatcaactcgcgccctggaagggatttttgaagcaactcatcgattgatttacggcgctaaggtaaatataaaatttttaagtgtataatgtgttaaactactgattctaattgtttgtgtattttaggatgactctggtcagagatacctggcctggtctggacacagtgcccgtgtcggagccgcgcgagatatggcccgcgctggagtttcaataccggagatcatgcaagctggtggctggaccaatgtaaatattgtcatgaactatatccgtaacctggatagtgaaacaggggcaatggtgcgcctgctggaagatggcgattagccattaacgcgtaaatgattgctataattatttgatatttatggtgacatatgagaaaggatttcaacatcgacggaaaatatgtagtgctgtctgtaagcactaatattcagtcgccagccgtcattgtcactgtaaagctgagcggcaataaaaagacagaataaaacgcacgggtgttgggtcgtttgttcggtcgagctcgcgaagctagcttggctgcaggtcgtcgaaattctaccgggtaggggaggcgcttttcccaaggcagtctggagcatgcgctttagcagccccgctgggcacttggcgctacacaagtggcctctggcctcgcacacattccacatccaccggtaggcgccaaccggctccgttctttggtggccccttcgcgccaccttctactcctcccctagtcaggaagttcccccccgccccgcagctcgcgtcgtgcaggacgtgacaaatggaagtagcacgtctcactagtctcgtgcagatggacagcaccgctgagcaatggaagcgggtaggcctttggggcagcggccaatagcagctttgctccttcgctttctgggctcagaggctgggaaggggtgggtccgggggcgggctcaggggcgggctcaggggcggggcgggcgcccgaaggtcctccggaggcccggcattctgcacgcttcaaaagcgcacgtctgccgcgctgttctcctcttcctcatctccgggcctttcgacctgcagcctgttgacaattaatcatcggcatagtatatcggcatagtataatacgacaaggtgaggaactaaaccatgggatcggccattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgatgatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgaggggatcaattctctagagctcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaaagaaccagctggggctcgactagagcttgcggaacccttaatataacttcgtataatgtatgctatacgaagttattaggtccctcgagaagagggtggttcaaagctggcaagaaaaagtaacccaaggaaaggacattggggtttaagggtataactcagttccaggatacttgccaagtgtgtgccaggccccggattaggtccccagcggctcacggtgcctttagtccacctaacccacaactggactctacagctcagctcccggttgccctgttaagcgtctgttccctctaactgaaaagatccaagcttgttgtacacgttctactgaatgcctgttactttcacaccatcttattaaagatgatctcccccccccccaaaaaaaaaaacaaaacaaaacaaaacaaaaacctgtaagtggagccagcttaagttgggaaccaactttcagaaagaaagttgttaaaatcgtgaaagtggttgtgtgaccttgtccagtgcttccatctcacttcatctctgctgcagatccgcgggcgtaaacgcttcgagatgttccgggagctgaatgaggccttagagttaaaggatgcccatgctacagaggagtctggagacagcagggctcactccaggtaagtggcctggggcagcgcctgcctgtggtgctctacccagacctccctccagctcagcctttgtagtgaaagataaaaaccccaccctgctagatgcttagggctgcaccctacgagaactgacttcttgactttttaggctctgttaaggggtatgagggacaaggtatggtgtcatgctcctataatctcagcagtaaggaagacaaagtcaggaggatttggggaagtttgaagccttcataaactatataaaactttaggccagctagggctagctacaatagcaagaccctgtctcatggtgatggtgatgatggtggtggtggtgacagttgtgataataatggcagtggtggtgatgatgatggtggtggtgatgatggtgatggtgaaagggaggataaactgattctcagaagtattccagtgtgttctgtgaatatccctacccatagtagaagccatcttaaattcctttttttcagcctccagcctagagccttccaagccttgatcaaggaggaaagcccaaactgctagctcccatcacttcatccctccccttttctgtcttcctatagctacctgaagaccaagaagggccagtctacttcccgccataaaaaaacaatggtcaagaaagtggggcctgactcagactgactgcctctgcatcccgtccccatcaccagcctccccctctccttgctgtcttatgacttcagggctgagacacaatcctcccggtcccttctgctgccttttttaccttgtagctagggctcagccccctctctgagtagtggttcctggcccaagttggggaataggttgatagttgtcaggtctctgctggcccagcgaaattctatccagccagttgttggaccctggcacctacaatgaaatctcaccctaccccacaccctgtaagattctatcttgggccctcatagggtccatatcctccagggcctactttccttccattctgcaaagcctgtctgcatttatccaccccccaccctgtctccctcttttttttttttttacccctttttatatatcaatttcctattttacaataaaattttgttatcacttatatggttttgagaggttgatatcagcataagctgtctgggcccccaggggcaggatgaatttgggaggtacccacctgagtccaggcagtctgttggagtcaggggtggggaacttgggttccagaagaggaacaaagccggggactgggtcagtctgtgggctgcatgacaacaagagagcagggtgactccattcataacttgggaaccaactgtccctcctccctcctgccaggactggcacatggtccttacccacccctacttctagggttgggctcctgctgtcctctggggagcctctcaccaaggattaagggatttaaatgtctgatatagcaaacctgagcctctggagtgaccatctgctccacaagaaaggatcagggtgcctgggttccacagggaagggggtggctgttcctggatgaagagacaagtgggaggcccgccagctggggtcccaggaaactgggagcagttaaggtgaggctaggggccttcccagcatccccaaactccgggcctcacgccaggcaagtgaatgaatcgaagctttgcactttgccaggaaatcctttgaaagggcttcttggagtagggaggagagtcagagttggggagcctaacaccctctcacccacccccttcctcagtgctgctggctcccagagagcgggactaggccaagctctccccatagcctgctgagccagtgccagtagggagagcagtcggtgagcacatggcttggccatggctcttggtaacagagagaggggtactgaggtccagaggcactttggggccactgtcctctgcctgtcccaggcttaaagagccagtgagagtcctttgccttgcccgagggtcctatacaagttgggagggagcagccccttagcctcaaaattttgtacttgtgagtaccaaatattttcgcagcagactagggtctatctttttaactgttgtgggaatgtgtgatgccagtcaaacccactacgcttttcccctgtttttgtcccacagctttactgagaactaaatccaaagaactgtgtttaagggccacaaatttgagcccttatctcaaaaagggaggaggggctaaaacaattgccaaatgaaaagctatctctgcatttgtgcctgtatggtaaacgttaagactgacagagaactactcaacaaaagctcctccacccaactcctttatttttttcagacagtaccttgctaactaagtagcccaggctaccctcaacattggtctctttatttagctttccaagtggagggagggatcgatgggctaccatacttccttaccttccccatttccctatcttttgtccttaattctgtccttgtacctcaagtcttcagaaagcaggtccattttggtacctcaaatcccttctctgagagctgatatcaatggacaaaggataatgaggtttttgcttctcctgcaagctcttctatgtaacttcacatcagtcacttacctcccaggttccacttccgggctgtttccattgatgcatcaactaggcatcgatccataaagtacttgacattcacagggaccctatgtctgctatgatgtctgcccaagaggtaaatacctcaggccttactctattgaggtctaaaatcaaacctcccaatctctaatcttgtaactctaacctacttttacattttaggtcaatatattttttattttttgtattttgagaaatggtggcgtgccagaccatgataagcaagacattgtaccagcaattaaccccccacccccaactccatagctgggtggtgatggagcacacctttaatcctggcacttgggacacagaggcaggcagagttcagcctagtaaacagagtgagttccagtccagccagggctatacagagaaaccctgtctcaaaaaaaacaaaacaaaaacaccaaacaaacaggaaaatcaaccccaggctggagagatggctcagcagttaagagcactgactgttcttccaaaggtcctgagttcaaatcccagcaagcacatggtggctcacaaccatctgacatctgatgccctctacaggtgtgtctgaagatagctacagtgtacttacatataaataataaatctttttttaaaaaaagaagaaaagaaaaatcaacccctattctggcttgatttttgtgatggtttcaagatcttgttgttgctgctgttttgttttgttaatttctgagatgatggagtcttgtacaacacaggctagctctaactcctgattctacctctacctccaaaatgctgagattataataaacaagtttcatgactcccagtacatatattgtttgtttggttggttggtttggtttgttttttttcgagacaaggttcctctgtatagccctggctgtcctggaactcactttgtagaccagtctggcctcgaactcagaaatctgcctgcctctgcctcccaagtgctgggaatacttttttaatttagaaattaatttaagaagcatgttgggtgccgggtgtggtggcgcactcctttaatcccagcactcgggaggcagaggcaggcggatttctgagttcgaggccagactggtctacaaagtgagttccaggacagccagggctacacagagaaaccctgtctcaaaaaaccaaaaaaaaaaaaaaaaaaaaaagcatgttggatctccaaatttgaggccagcctggtctacagcacaagttccaggacaaacacagacaaaccctatttcaaacatacatagggtcaggaggcaaaggcaggtagatctctgtgagtaccagtctcgcttgcctggtctatatagtgagttctaggtattcaggaaataacctagataatgttagccccaagtgctgtcattatttaagagagagagaaaataaaggaacctaggggctagagaagtggctcaatgattatgaaagagtacaagctgctcttccagaggaccagggttctgatcccagcacccatgtcaagtcactcacaaatccatatcaccaagagattcaccctcggcgcgccactcttcgcgacagctagatctcatcgcctaggatcgcccgggttgattcgaggctgctaacaaatcgagtcgagcatcgagcagtgtggttttcaagaggaagcaaaaagcctctccacccaggcctggaatgtttccacccaatgtcgagcagtgtggttttgcaagaggaagcaaaaagcctctccacccaggcctggaatgtttccacccaatgtcgagcaaaccccgcccagcgtcttgtcattggcgaattcgaacacgcagatgcagtcggggcggcgcggtcccaggtccacttcgcatattaaggtgacgcgtgtggcctcgaacaccgagcgaccctgcagcgacccgcttaacagcgtcaacagcgtgccgcagatcttggtggcgtgaaactcccgcacctcttcggccagcgccttgtagaagcgcgtgccatggatcctgatgatgttgttgattcttctaaatcttttgtgatggaaaacttttcttcgtaccacgggactaaacctggttatgtagattccattcaaaaaggtatacaaaagccaaaatctggtacacaaggaaattatgacgatgattggaaagggttttatagtaccgacaataaatacgacgctgcgggatactctgtagataatgaaaacccgctctctggaaaagctggaggcgtggtcaaagtgacgtatccaggactgacgaaggttctcgcactaaaagtggataatgccgaaactattaagaaagagttaggtttaagtctcactgaaccgttgatggagcaagtcggaacggaagagtttatcaaaaggttcggtgatggtgcttcgcgtgtagtgctcagccttcccttcgctgaggggagttctagcgttgaatatattaataactgggaacaggcgaaagcgttaagcgtagaacttgagattaattttgaaacccgtggaaaacgtggccaagatgcgatgtatgagtatatggctcaagcctgtgcaggaaatcgtgtcaggcgatctctttgtgaaggaaccttacttctgtggtgtgacataattggacaaactacctacagagatttaaagctctaaggtaaatataaaatttttaagtgtataatgtgttaaactactgattctaattgtttgtgtattttagattccaacctatggaactgatgaatgggagcagtggtggaatgcagatcctagagctcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaaagaaccagcccgggcggtggagctccaattcgccctatagtgagtcgtattacaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggaaattgtaagcg

SEQ ID NO: 13 - protein, 396 aa, Artificial Sequence
  Description of Artificial Sequence Synthetic polypeptide
MTAMEESQSDISLELPLSQETFSGLWKLLPPEDILSPLPSQAMDDLMLSPDDIEQWFTEDPGPDEAPRMPEAAPPVAPAPAAPTPAAPAPAPSWPLSSSVPSQKTYQGSYGFRLGFLHSGTAKSVTCTYSPALNKMFCQLAKTCPVQLWVDSTPPPGTRVRAMAIYKQSQHMTEVVRRCPHHERCSDSDGLAPPQHLIRVEGNLRVEYLDDRNTFRHSVVVPCEPPEVGSDCTTIHYNYMCNSSCMGGMNRRPILTIITLEDSSGNLLGRNSFEVRVCACPGRDRRTEEENLRKKGEPHHELPPGSTKRALPNNTSSSPQPKKKPLDGEYFTLQIRGRKRFEMFRELNEALELKDAHATEESGDSRAHSSYLKTKKGQSTSRHKKTMVKKVGPDSD

SEQ ID NO: 14 - protein, 396 aa, Artificial Sequence
  Description of Artificial Sequence Synthetic polypeptide
MTAMEESQSDISLELPLSQETFSGLWKLLPPEDILSPLPSQAMDDLMLSPDDIEQWFTEDPGPDEAPRMPEAAPRVAPAPAAPTPAAPAPAPSWPLSSSVPSQKTYQGSYGFRLGFLHSGTAKSVTCTYSPALNKMFCQLAKTCPVQLWVDSTPPPGTRVRAMAIYKQSQHMTEVVRRCPHHERCSDSDGLAPPQHLIRVEGNLRVEYLDDRNTFRHSVVVPCEPPEVGSDCTTIHYNYMCNSSCMGGMNRRPILTIITLEDSSGNLLGRNSFEVRVCACPGRDRRTEEENLRKKGEPHHELPPGSTKRALPNNTSSSPQPKKKPLDGEYFTLQIRGRKRFEMFRELNEALELKDAHATEESGDSRAHSSYLKTKKGQSTSRHKKTMVKKVGPDSD

SEQ ID NO: 15 - DNA, 157 bp, Mus musculus
tttcccctcccacgtgctcaccctggctaaagttctgtagcttcagttcattgggaccatcctggctgtaggtagcgactacagttagggggcacctagcattcaggccctcatcctcctccttcccagcagggtgtcacgcttctccgaagactgg

SEQ ID NO: 16 - DNA, 83 bp, Mus musculus
atgactgccatggaggagtcacagtcggatatcagcctcgagctccctctgagccaggagacattttcaggcttatggaaact

SEQ ID NO: 17 - protein, 27 aa, Mus musculus
MTAMEESQSDISLELPLSQETFSGLWK

SEQ ID NO: 18 - DNA, 22 bp, Mus musculus
acttcctccagaagatatcctg

SEQ ID NO: 19 - protein, 8 aa, Mus musculus
LLPPEDIL

SEQ ID NO: 20 - DNA, 261 bp, Mus musculus
ccatcacctcactgcatggacgatctgttgctgccccaggatgttgaggagttttttgaaggcccaagtgaagccctccgagtgtcaggagctcctgcagcacaggaccctgtcaccgagacccctgggccagtggcccctgccccagccactccatggcccctgtcatcttttgtcccttctcaaaaaacttaccagggcaactatggcttccacctgggcttcctgcagtctgggacagccaagtctgttatgtgcacg

SEQ ID NO: 21 - protein, 87 aa, Mus musculus
PSPHCMDDLLLPQDVEEFFEGPSEALRVSGAPAAQDPVTETPGPVAPAPATPWPLSSFVPSQKTYQGNYGFHLGFLQSGTAKSVMCT

SEQ ID NO: 22 - DNA, 184 bp, Mus musculus
tactctcctcccctcaataagctattctgccagctggcgaagacgtgccctgtgcagttgtgggtcagcgccacacctccagctgggagccgtgtccgcgccatggccatctacaagaagtcacagcacatgacggaggtcgtgagacgctgcccccaccatgagcgctgctccgatggtgatg

SEQ ID NO: 23 - protein, 61 aa, Mus musculus
YSPPLNKLFCQLAKTCPVQLWVSATPPAGSRVRAMAIYKKSQHMTEVVRRCPHHERCSDGD

SEQ ID NO: 24 - DNA, 113 bp, Mus musculus
gcctggctcctccccagcatcttatccgggtggaaggaaatttgtatcccgagtatctggaagacaggcagacttttcgccacagcgtggtggtaccttatgagccacccgag

SEQ ID NO: 25 - protein, 38 aa, Mus musculus
GLAPPQHLIRVEGNLYPEYLEDRQTFRHSVVVPYEPPE

SEQ ID NO: 26 - DNA, 110 bp, Mus musculus
gccggctctgagtataccaccatccactacaagtacatgtgtaatagctcctgcatggggggcatgaaccgccgacctatccttaccatcatcacactggaagactccag

SEQ ID NO: 27 - protein, 36 aa, Mus musculus
AGSEYTTIHYKYMCNSSCMGGMNRRPILTIITLEDS

SEQ ID NO: 28 - DNA, 137 bp, Mus musculus
tgggaaccttctgggacgggacagctttgaggttcgtgtttgtgcctgccctgggagagaccgccgtacagaagaagaaaatttccgcaaaaaggaagtcctttgccctgaactgcccccagggagcgcaaagagag

SEQ ID NO: 29 - protein, 46 aa, Mus musculus
SGNLLGRDSFEVRVCACPGRDRRTEEENFRKKEVLCPELPPGSAKR

SEQ ID NO: 30 - DNA, 74 bp, Mus musculus
cgctgcccacctgcacaagcgcctctcccccgcaaaagaaaaaaccacttgatggagagtatttcaccctcaag

SEQ ID NO: 31 - protein, 25 aa, Mus musculus
ALPTCTSASPPQKKKPLDGEYFTLK

SEQ ID NO: 32 - DNA, 107 bp, Mus musculus
atccgcgggcgtaaacgcttcgagatgttccgggagctgaatgaggccttagagttaaaggatgcccatgctacagaggagtctggagacagcagggctcactccag

SEQ ID NO: 33 - protein, 35 aa, Mus musculus
IRGRKRFEMFRELNEALELKDAHATEESGDSRAHS

SEQ ID NO: 34 - DNA, 524 bp, Mus musculus
ctacctgaagaccaagaagggccagtctacttcccgccataaaaaaacaatggtcaagaaagtggggcctgactcagactgactgcctctgcatcccgtccccatcaccagcctccccctctccttgctgtcttatgacttcagggctgagacacaatcctcccggtcccttctgctgccttttttaccttgtagctagggctcagccccctctctgagtagtggttcctggcccaagttggggaataggttgatagttgtcaggtctctgctggcccagcgaaattctatccagccagttgttggaccctggcacctacaatgaaatctcaccctaccccacaccctgtaagattctatcttgggccctcatagggtccatatcctccagggcctactttccttccattctgcaaagcctgtctgcatttatccaccccccaccctgtctccctcttttttttttttttacccctttttatatatcaatttcctattttacaataaaattttgttatcactta

SEQ ID NO: 35 - protein, 27 aa, Mus musculus
SYLKTKKGQSTSRHKKTMVKKVGPDSD

SEQ ID NO: 36 - DNA, 114 bp, Homo sapiens
ctcaaaagtctagagccaccgtccagggagcaggtagctgctgggctccggggacactttgcgttcgggctgggagcgtgctttccacgacggtgacacgcttccctggattgg

SEQ ID NO: 37 - DNA, 102 bp, Homo sapiens
cagccagactgccttccgggtcactgccatggaggagccgcagtcagatcctagcgtcgagccccctctgagtcaggaaacattttcagacctatggaaact

SEQ ID NO: 38 - protein, 24 aa, Homo sapiens
MEEPQSDPSVEPPLSQETFSDLWK

SEQ ID NO: 39 - DNA, 22 bp, Homo sapiens
acttcctgaaaacaacgttctg

SEQ ID NO: 40 - protein, 8 aa, Homo sapiens
LLPENNVL

SEQ ID NO: 41 - DNA, 279 bp, Homo sapiens
tcccccttgccgtcccaagcaatggatgatttgatgctgtccccggacgatattgaacaatggttcactgaagacccaggtccagatgaagctcccagaatgccagaggctgctccccccgtggcccctgcaccagcagctcctacaccggcggcccctgcaccagccccctcctggcccctgtcatcttctgtcccttcccagaaaacctaccagggcagctacggtttccgtctgggcttcttgcattctgggacagccaagtctgtgacttgcacg

SEQ ID NO: 42 - protein, 93 aa, Homo sapiens
SPLPSQAMDDLMLSPDDIEQWFTEDPGPDEAPRMPEAAPPVAPAPAAPTPAAPAPAPSWPLSSSVPSQKTYQGSYGFRLGFLHSGTAKSVTCT

SEQ ID NO: 43 - DNA, 184 bp, Homo sapiens
tactcccctgccctcaacaagatgttttgccaactggccaagacctgccctgtgcagctgtgggttgattccacacccccgcccggcacccgcgtccgcgccatggccatctacaagcagtcacagcacatgacggaggttgtgaggcgctgcccccaccatgagcgctgctcagatagcgatg

SEQ ID NO: 44 - protein, 61 aa, Homo sapiens
YSPALNKMFCQLAKTCPVQLWVDSTPPPGTRVRAMAIYKQSQHMTEVVRRCPHHERCSDSD

SEQ ID NO: 45 - DNA, 113 bp, Homo sapiens
gtctggcccctcctcagcatcttatccgagtggaaggaaatttgcgtgtggagtatttggatgacagaaacacttttcgacatagtgtggtggtgccctatgagccgcctgag

SEQ ID NO: 46 - protein, 38 aa, Homo sapiens
GLAPPQHLIRVEGNLRVEYLDDRNTFRHSVVVPYEPPE

SEQ ID NO: 47 - DNA, 110 bp, Homo sapiens
gttggctctgactgtaccaccatccactacaactacatgtgtaacagttcctgcatgggcggcatgaaccggaggcccatcctcaccatcatcacactggaagactccag

SEQ ID NO: 48 - protein, 36 aa, Homo sapiens
VGSDCTTIHYNYMCNSSCMGGMNRRPILTIITLEDS

SEQ ID NO: 49 - DNA, 137 bp, Homo sapiens
tggtaatctactgggacggaacagctttgaggtgcgtgtttgtgcctgtcctgggagagaccggcgcacagaggaagagaatctccgcaagaaaggggagcctcaccacgagctgcccccagggagcactaagcgag

SEQ ID NO: 50 - protein, 46 aa, Homo sapiens
SGNLLGRNSFEVRVCACPGRDRRTEEENLRKKGEPHHELPPGSTKR

SEQ ID NO: 51 - DNA, 74 bp, Homo sapiens
cactgcccaacaacaccagctcctctccccagccaaagaagaaaccactggatggagaatatttcacccttcag

SEQ ID NO: 52 - protein, 25 aa, Homo sapiens
ALPNNTSSSPQPKKKPLDGEYFTLQ

SEQ ID NO: 53 - DNA, 107 bp, Homo sapiens
atccgtgggcgtgagcgcttcgagatgttccgagagctgaatgaggccttggaactcaaggatgcccaggctgggaaggagccaggggggagcagggctcactccag

SEQ ID NO: 54 - protein, 35 aa, Homo sapiens
IRGRERFEMFRELNEALELKDAQAGKEPGGSRAHS

SEQ ID NO: 55 - DNA, 1270 bp, Homo sapiens
ccacctgaagtccaaaaagggtcagtctacctcccgccataaaaaactcatgttcaagacagaagggcctgactcagactgacattctccacttcttgttccccactgacagcctcccacccccatctctccctcccctgccattttgggttttgggtctttgaacccttgcttgcaataggtgtgcgtcagaagcacccaggacttccatttgctttgtcccggggctccactgaacaagttggcctgcactggtgttttgttgtggggaggaggatggggagtaggacataccagcttagattttaaggtttttactgtgagggatgtttgggagatgtaagaaatgttcttgcagttaagggttagtttacaatcagccacattctaggtaggggcccacttcaccgtactaaccagggaagctgtccctcactgttgaattttctctaacttcaaggcccatatctgtgaaatgctggcatttgcacctacctcacagagtgcattgtgagggttaatgaaataatgtacatctggccttgaaaccaccttttattacatggggtctagaacttgacccccttgagggtgcttgttccctctccctgttggtcggtgggttggtagtttctacagttgggcagctggttaggtagagggagttgtcaagtctctgctggcccagccaaaccctgtctgacaacctcttggtgaaccttagtacctaaaaggaaatctcaccccatcccacaccctggaggatttcatctcttgtatatgatgatctggatccaccaagacttgttttatgctcagggtcaatttcttttttcttttttttttttttttttctttttctttgagactgggtctcgctttgttgcccaggctggagtggagtggcgtgatcttggcttactgcagcctttgcctccccggctcgagcagtcctgcctcagcctccggagtagctgggaccacaggttcatgccaccatggccagccaacttttgcatgttttgtagagatggggtctcacagtgttgcccaggctggtctcaaactcctgggctcaggcgatccacctgtctcagcctcccagagtgctgggattacaattgtgagccaccacgtccagctggaagggtcaacatcttttacattctgcaagcacatctgcattttcaccccacccttcccctccttctccctttttatatcccatttttatatcgatctcttattttacaataaaactttgctgcca

SEQ ID NO: 56 - protein, 27 aa, Homo sapiens
SHLKSKKGQSTSRHKKLMFKTEGPDSD

(19) United States
(12) Patent Application Publication
(10) Pub. No.: US 2021/0403945 A1
(43) Pub. Date: Dec. 30, 2021

(54) COMPOSITIONS AND METHODS UTILIZING GENETICALLY-MODIFIED ANIMALS AND CELLS

(71) Applicant: PMV Pharmaceuticals, Inc., Cranbury, NJ (US)

(72) Inventors: Arnold Levine, Doylestown, PA (US); David Mack, Menlo Park, CA (US); Anna Puzio-Kuter, Hillsborough, NJ (US); Lizhong Xu, Jamison, PA (US); Thomas W. Davis, South Orange, NJ (US); Melissa Dumble, Watchung, NJ (US)

(21) Appl. No.: 17/356,920

(22) Filed: Jun. 24, 2021

Related U.S. Application Data
(60) Provisional application No. 63/043,412, filed on Jun. 24, 2020.

(51) Int. Cl. C12N 15/85; C07K 14/82; A01K 67/027

(52) U.S. Cl. CPC C12N 15/8509; C07K 14/82; A01K 67/0276; C12N 2830/003; A01K 2217/075; A01K 2227/105; A01K 2267/0331; A01K 2217/05

(57) ABSTRACT

Provided herein are compositions and methods for studying cancer therapeutics and etiology, for example, mouse cancer models, cancer cell lines, and uses thereof. Human p53 knock-in (Hupki) mice with a Y220 (e.g., Y220C, Y220H, or Y220S) mutation in p53 are provided. These Hupki-Y220 mice can be used, for example, to examine tumorigenesis in different tissues, investigate mechanisms of gain of function, develop mouse models of cancer, generate cancer cell lines that can be implanted into recipient mice, and test potential therapeutics.

### CROSS REFERENCE

This application claims the benefit of U.S. Provisional Patent Application No. 63/043,412, filed on Jun. 24, 2020, which is incorporated herein by reference in its entirety.

### BACKGROUND

Cancer, an uncontrolled proliferation of cells, is a multifactorial disease characterized by tumor formation, growth, and in some instances, metastasis. Replication of cells with cancer-promoting alterations can be inhibited by an elaborate tumor suppression network. A central component of this tumor suppression network is the tumor suppressor p53, and mutations of p53 are implicated in the progression of cancer.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### SUMMARY OF THE INVENTION

Provided herein is an engineered non-human mammalian cell comprising a nucleic acid that encodes a p53 protein, wherein the nucleic acid comprises a sequence with at least 90% sequence identity to exon 6 of human TP53, wherein the sequence with at least 90% sequence identity to exon 6 of human TP53 encodes an amino acid substitution at position 220 relative to human p53.

Provided herein is an assay comprising: (a) contacting a population of engineered non-human mammalian cells with a therapeutic agent, wherein the engineered non-human mammalian cells each comprise a nucleic acid that encodes a p53 protein, wherein the nucleic acid comprises a sequence with at least 90% sequence identity to exon 6 of human TP53, wherein the sequence with at least 90% sequence identity to exon 6 of human TP53 encodes an amino acid substitution at position 220 relative to human p53; and (b) after the contacting, observing an effect of the therapeutic agent on the population of engineered non-human mammalian cells.

Provided herein is a method of evaluating a therapeutic agent, comprising administering a therapeutically-effective amount of the therapeutic agent to a subject with a cancer, wherein the cancer comprises an engineered non-human mammalian cell, wherein the engineered non-human mammalian cell comprises a nucleic acid that encodes a p53 protein, wherein the nucleic acid comprises a sequence with at least 90% sequence identity to exon 6 of human TP53, wherein the sequence with at least 90% sequence identity to exon 6 of human TP53 encodes an amino acid substitution at position 220 relative to human p53. Further provided herein is a non-human animal comprising the engineered non-human mammalian cell comprising a nucleic acid that encodes a p53 protein, wherein the nucleic acid comprises a sequence with at least 90% sequence identity to exon 6 of human TP53, wherein the sequence with at least 90% sequence identity to exon 6 of human TP53 encodes an amino acid substitution at position 220 relative to human p53.

### INCORPORATION BY REFERENCE

Each patent, publication, and non-patent literature cited in the application is hereby incorporated by reference in its entirety as if each was incorporated by reference individually.

### BRIEF DESCRIPTION OF THE DRAWINGS

FIG. 1 provides a linearized diagram of a targeting vector used for generating for Hupki-Y220C mice.

FIG. 2 provides a survival plot of Hupki-Y220C mice across 516 days.

FIG. 3 illustrates the sensitivity of five Hupki-Y220C lymphoma and sarcoma cell lines to Compound 1. IC50 values were calculated from a 5-day MTT assay.

FIG. 4 shows anti-cancer activity of Compound 1 in the MT373 syngeneic model of soft tissue sarcoma. 2Q7D signifies twice daily dosing, once per week.

FIG. 5 shows anti-cancer activity of Compound 1 in the MT245 syngeneic model of soft tissue sarcoma. Q7D signifies dosing once per week.

FIG. 6A and FIG. 6B show the effects of Compound 1 on the relative intra-tumoral expression of genes that are upstream or downstream of p53. FIG. 6A shows expression of Bbc3, Birc5, Cdnk1a, Chek1, Sesn2, and Zmat3. FIG. 6B shows expression of Ccng1, Cdc25c, Egr1, and IL6.

FIG. 7 shows the effects of Compound 1 on the relative intra-tumoral expression of four genes in the NF-κB signaling pathway.

FIG. 8 shows the effects of Compound 1 and/or anti-PD-1 antibody on tumor volume in mice bearing MT373 tumors. 2Q7D signifies twice daily, once per week treatment. Q3D signifies treatment every 3 days.

FIG. 9 shows the effects of Compound 1 and/or anti-PD-1 antibody on tumor volume in mice bearing MT245 tumors. Q7D signifies once daily, once per week treatment. Q3D signifies treatment every 3 days.

FIG. 10 shows the effects of Compound 1 and/or anti-CTLA4 antibody on tumor volume in mice bearing MT373 tumors. Q7D signifies once daily, once per week treatment. Q3D signifies treatment every 3 days.

FIG. 11A and FIG. 11B demonstrate the effects of treatment with Compound 1 on populations of intra-tumor leukocytes. FIG. 11A shows the proportion of CD45+ cells that are total T cells (CD3+), CD4+ T cells, CD8+T cells, and T effector cells. FIG. 11B shows the proportion of total CD45+ cells that are m-MDSCs, macrophages, and NKT cells.

FIG. 12A and FIG. 12B show the effects of treatment with Compound 1 on intra-tumor cytokine concentrations over time. FIG. 12A shows the concentrations of MCP-1. GM-CSF, VEGF, IFNγ, IFNB1, and LIF. FIG. 12B shows the concentrations of M-CSF, IL-1a, and RANTES.

FIG. 13 shows the effects of Compound 1, anti-PD-1 antibody, and CD8+ T cell depletion on growth of MT373 tumors.

### DETAILED DESCRIPTION

Provided herein are compositions and methods for studying cancer therapeutics and etiology, for example, mouse cancer models, cancer cell lines, and uses thereof.

Cancer is a collection of related diseases characterized by uncontrolled proliferation of cells with the potential to metastasize throughout the body. Cancer can be classified into five broad categories including, for example: carcinomas, which can arise from cells that cover internal and external parts of the body such as the lung, breast, and colon; sarcomas, which can arise from cells that are located in bone, cartilage, fat, connective tissue, muscle, and other supportive tissues; lymphomas, which can arise in the lymph nodes and immune system tissues; leukemia, which can arise in the bone marrow and accumulate in the bloodstream; and adenomas, which can arise in the thyroid, the pituitary gland, the adrenal gland, and other glandular tissues.

Although cancers can develop in virtually any of the body's tissues, and contain unique features, the basic processes that cause cancer can be similar in all forms of the disease. Cancer begins when a cell breaks free from the normal restraints on cell division and begins to grow and divide out of control. Genetic mutations in the cell can preclude the ability of the cell to repair damaged DNA or initiate apoptosis, and can result in uncontrolled growth and division of cells.

The ability of tumor cell populations to multiply can be determined not only by the rate of cell proliferation but also by the rate of cell attrition. Programmed cell death, or apoptosis, represents a major mechanism of cellular attrition. Cancer cells can evade apoptosis through a variety of strategies, for example, through the suppression of p53 function, thereby suppressing expression of pro-apoptotic proteins.

Oncogenes and tumor suppressor genes can regulate the proliferation of cells. Genetic mutations can result in abnormal function of oncogenes and tumor suppressor genes, potentially facilitating uncontrolled cell division. Whereas oncogenes assist in cellular growth, tumor suppressor genes slow cell division to allow for repair of damaged DNA and activating apoptosis. Cellular oncogenes that can be mutated in cancer include, for example, Cdk1, Cdk2, Cdk3, Cdk4, Cdk6, EGFR, PDGFR, VEGF, HER2, Raf kinase, K-Ras, and myc. Tumor suppressor genes that can be mutated in cancer include, for example, BRCA1, BRCA2, cyclin-dependent kinase inhibitor 1C, Retinoblastoma protein (Rb or Rb1), PTEN, p16, p27, p53, and p73.

Provided herein are compositions and methods for studying cancer therapeutics and etiology, for example, mouse cancer models, cancer cell lines, and uses thereof. Human p53 knock-in (Hupki) mice with a Y220 (e.g., Y220C) mutation in p53, cells derived therefrom, and uses thereof are provided. These Hupki-Y220 mice can be used, for example, to examine tumorigenesis in different tissues, investigate mechanisms of gain of function, develop mouse models of cancer, generate cancer cell lines that can be implanted into recipient mice, and test potential therapeutics and combination therapies.

### Tumor Suppressor p53

The tumor suppressor protein p53 is a 393 amino acid transcription factor that can regulate cell growth in response to cellular stresses including, for example, UV radiation, hypoxia, oncogene activation, and DNA damage. p53 has various mechanisms for inhibiting the progression of cancer including, for example, initiation of apoptosis, maintenance of genomic stability, cell cycle arrest, induction of senescence, and inhibition of angiogenesis. Due to the critical role of p53 in tumor suppression, p53 is inactivated in many cancers either by direct mutation or through perturbation of associated signaling pathways involved in tumor suppression. Homozygous loss of the p53 gene function occurs in many types of cancer, including carcinomas of the breast, colon, and lung. The presence of certain p53 mutations in several types of human cancer can correlate with less favorable patient prognosis.

In the absence of stress signals, p53 levels are maintained at low levels via the interaction of p53 with Mdm2, an E3 ubiquitin ligase. In an unstressed cell, Mdm2 can target p53 for degradation by the proteasome. Under stress conditions, the interaction between Mdm2 and p53 is disrupted, and p53 accumulates. The critical event leading to the activation of p53 is phosphorylation of the N-terminal domain of p53 by protein kinases, thereby transducing upstream stress signals. The phosphorylation of p53 leads to a conformational change, which can promote DNA binding by p53 and allow transcription of downstream effectors. The activation of p53 can induce, for example, the intrinsic apoptotic pathway, the extrinsic apoptotic pathway, cell cycle arrest, senescence, and DNA repair. p53 can activate proteins involved in the above pathways including, for example, Fas/Apol, KILLER/DRS, Bax, Puma, Noxa, Bid, caspase-3, caspase-6, caspase-7, caspase-8, caspase-9, and p21 (WAF1). Additionally, p53 can repress the transcription of a variety of genes including, for example, c-MYC, Cyclin B, VEGF, RAD51, and hTERT.

Each chain of the p53 tetramer is composed of several functional domains including the transactivation domain (amino acids 1-100), the DNA-binding domain (amino acids 101-306), and the tetramerization domain (amino acids 307-355), which are highly mobile and largely unstructured. Most p53 cancer mutations are located in the DNA-binding core domain of the protein, which contains a central (3-sandwich of anti-parallel β-sheets that serves as a basic scaffold for the DNA-binding surface. The DNA-binding surface is composed of two β-turn loops, L2 and L3, which are stabilized by a zinc ion, for example, at Arg175 and Arg248, and a loop-sheet-helix motif. Altogether, these structural elements form an extended DNA-binding surface that is rich in positively-charged amino acids, and makes specific contact with various p53 response elements.

Mutations in p53 located in the DNA-binding domain of the protein or periphery of the DNA-binding surface can result in aberrant protein folding, which can interfere with DNA recognition and binding. Mutations in p53 can occur, for example, at amino acids Va1143, His168, Arg175, Tyr220, Gly245, Arg248, Arg249, Phe270, Arg273, and Arg282. p53 mutations that can abrogate the activity of p53 include, for example, R175H, Y220C, Y220S, Y220H, G245S, R248Q, R248W, R273H, and R282W. These p53 mutations can either distort the structure of the DNA-binding site or thermodynamically destabilize the folded protein at body temperature.

Several human isoforms of p53 exist. Non-limiting examples of amino acid sequences of human p53 proteins are provided in Table 1. In some embodiments, SEQ ID NO: 1 provides the canonical p53 sequence, and a reference herein to an amino acid number, e.g., Y220, refers to the corresponding position in SEQ ID NO: 1. In some embodiments, a p53 protein includes arginine instead of proline at position 72 (P72R).

TABLE 1

| SEQ | | |
|---|---|---|
| ID | | |
| NO: | Description | Sequence |
| 1 | Human p53 | MEEPQSDPSVEPPLSQETFSDLWKLLPENNVLSPLPSQAMDDL |
| | isoform 1 | MLSPDDIEQWFTEDPGPDEAPRMPEAAPPVAPAPAAPTPAAPA |
| | | PAPSWPLSSSVPSQKTYQGSYGFRLGFLHSGTAKSVTCTYSPA |
| | | LNKMFCQLAKTCPVQLWVDSTPPPGTRVRAMAIYKQSQHMT |
| | | EVVRRCPHHERCSDSDGLAPPQHLIRVEGNLRVEYLDDRNTFR |
| | | HSVVVPYEPPEVGSDCTTIHYNYMCNSSCMGGMNRRPILTIITL |
| | | EDSSGNLLGRNSFEVRVCACPGRDRRTEEENLRKKGEPHHELP |
| | | PGSTKRALPNNTSSSPQPKKKPLDGEYFTLQIRGRERFEMFREL |
| | | NEALELKDAQAGKEPGGSRAHSSHLKSKKGQSTSRHKKLMFK |
| | | TEGPDSD |
| 10 | Human p53 | MEEPQSDPSVEPPLSQETFSDLWKLLPENNVLSPLPSQAMDDL |
| | isoform 1 | MLSPDDIEQWFTEDPGPDEAPRMPEAAPRVAPAPAAPTPAAPA |
| | with P72R | PAPSWPLSSSVPSQKTYQGSYGFRLGFLHSGTAKSVTCTYSPA |
| | substitution | LNKMFCQLAKTCPVQLWVDSTPPPGTRVRAMAIYKQSQHMT |
| | | EVVRRCPHHERCSDSDGLAPPQHLIRVEGNLRVEYLDDRNTFR |
| | | HSVVVPYEPPEVGSDCTTIHYNYMCNSSCMGGMNRRPILTIITL |
| | | EDSSGNLLGRNSFEVRVCACPGRDRRTEEENLRKKGEPHHELP |
| | | PGSTKRALPNNTSSSPQPKKKPLDGEYFTLQIRGRERFEMFREL |
| | | NEALELKDAQAGKEPGGSRAHSSHLKSKKGQSTSRHKKLMFK |
| | | TEGPDSD |
| 2 | Human p53 | MEEPQSDPSVEPPLSQETFSDLWKLLPENNVLSPLPSQAMDDL |
| | isoform 2 | MLSPDDIEQWFTEDPGPDEAPRMPEAAPPVAPAPAAPTPAAPA |
| | | PAPSWPLSSSVPSQKTYQGSYGFRLGFLHSGTAKSVTCTYSPA |
| | | LNKMFCQLAKTCPVQLWVDSTPPPGTRVRAMAIYKQSQHMT |
| | | EVVRRCPHHERCSDSDGLAPPQHLIRVEGNLRVEYLDDRNTFR |
| | | HSVVVPYEPPEVGSDCTTIHYNYMCNSSCMGGMNRRPILTIITL |
| | | EDSSGNLLGRNSFEVRVCACPGRDRRTEEENLRKKGEPHHELP |
| | | PGSTKRALPNNTSSSPQPKKKPLDGEYFTLQDQTSFQKENC |
| 3 | Human p53 | MEEPQSDPSVEPPLSQETFSDLWKLLPENNVLSPLPSQAMDDL |
| | isoform 3 | MLSPDDIEQWFTEDPGPDEAPRMPEAAPPVAPAPAAPTPAAPA |
| | | PAPSWPLSSSVPSQKTYQGSYGFRLGFLHSGTAKSVTCTYSPA |
| | | LNKMFCQLAKTCPVQLWVDSTPPPGTRVRAMAIYKQSQHMT |
| | | EVVRRCPHHERCSDSDGLAPPQHLIRVEGNLRVEYLDDRNTFR |
| | | HSVVVPYEPPEVGSDCTTIHYNYMCNSSCMGGMNRRPILTIITL |
| | | EDSSGNLLGRNSFEVRVCACPGRDRRTEEENLRKKGEPHHELP |
| | | PGSTKRALPNNTSSSPQPKKKPLDGEYFTLQMLLDLRWCYFLI |
| | | NSS |
| 4 | Human p53 | MDDLMLSPDDIEQWFTEDPGPDEAPRMPEAAPPVAPAPAAPTP |
| | isoform 4 | AAPAPAPSWPLSSSVPSQKTYQGSYGFRLGFLHSGTAKSVTCT |
| | | YSPALNKMFCQLAKTCPVQLWVDSTPPPGTRVRAMAIYKQSQ |
| | | HMTEVVRRCPHHERCSDSDGLAPPQHLIRVEGNLRVEYLDDR |
| | | NTFRHSVVVPYEPPEVGSDCTTIHYNYMCNSSCMGGMNRRPIL |
| | | TIITLEDSSGNLLGRNSFEVRVCACPGRDRRTEEENLRKKGEPH |
| | | HELPPGSTKRALPNNTSSSPQPKKKPLDGEYFTLQIRGRERFEM |
| | | FRELNEALELKDAQAGKEPGGSRAHSSHLKSKKGQSTSRHKK |
| | | LMFKTEGPDSD |
| 5 | Human p53 | MDDLMLSPDDIEQWFTEDPGPDEAPRMPEAAPPVAPAPAAPTP |
| | isoform 5 | AAPAPAPSWPLSSSVPSQKTYQGSYGFRLGFLHSGTAKSVTCT |
| | | YSPALNKMFCQLAKTCPVQLWVDSTPPPGTRVRAMAIYKQSQ |
| | | HMTEVVRRCPHHERCSDSDGLAPPQHLIRVEGNLRVEYLDDR |
| | | NTFRHSVVVPYEPPEVGSDCTTIHYNYMCNSSCMGGMNRRPIL |
| | | TIITLEDSSGNLLGRNSFEVRVCACPGRDRRTEEENLRKKGEPH |
| | | HELPPGSTKRALPNNTSSSPQPKKKPLDGEYFTLQDQTSFQKE |
| | | NC |
| 6 | Human p53 | MDDLMLSPDDIEQWFTEDPGPDEAPRMPEAAPPVAPAPAAPTP |
| | isoform 6 | AAPAPAPSWPLSSSVPSQKTYQGSYGFRLGFLHSGTAKSVTCT |
| | | YSPALNKMFCQLAKTCPVQLWVDSTPPPGTRVRAMAIYKQSQ |
| | | HMTEVVRRCPHHERCSDSDGLAPPQHLIRVEGNLRVEYLDDR |
| | | NTFRHSVVVPYEPPEVGSDCTTIHYNYMCNSSCMGGMNRRPIL |
| | | TIITLEDSSGNLLGRNSFEVRVCACPGRDRRTEEENLRKKGEPH |
| | | HELPPGSTKRALPNNTSSSPQPKKKPLDGEYFTLQMLLDLRWC |
| | | YFLINSS |
| 7 | Human p53 | MFCQLAKTCPVQLWVDSTPPPGTRVRAMAIYKQSQHMTEVV |
| | isoform 7 | RRCPHHERCSDSDGLAPPQHLIRVEGNLRVEYLDDRNTFRHSV |
| | | VVPYEPPEVGSDCTTIHYNYMCNSSCMGGMNRRPILTIITLEDS |
| | | SGNLLGRNSFEVRVCACPGRDRRTEEENLRKKGEPHHELPPGS |
| | | TKRALPNNTSSSPQPKKKPLDGEYFTLQIRGRERFEMFRELNEA |
| | | LELKDAQAGKEPGGSRAHSSHLKSKKGQSTSRHKKLMFKTEG |
| | | PDSD |
| 8 | Human p53 | MFCQLAKTCPVQLWVDSTPPPGTRVRAMAIYKQSQHMTEVV |
| | isoform 8 | RRCPHHERCSDSDGLAPPQHLIRVEGNLRVEYLDDRNTFRHSV |
| | | VVPYEPPEVGSDCTTIHYNYMCNSSCMGGMNRRPILTIITLEDS |
| | | SGNLLGRNSFEVRVCACPGRDRRTEEENLRKKGEPHHELPPGS |
| | | TKRALPNNTSSSPQPKKKPLDGEYFTLQDQTSFQKENC |
| 9 | Human p53 | MFCQLAKTCPVQLWVDSTPPPGTRVRAMAIYKQSQHMTEVV |
| | isoform 9 | RRCPHHERCSDSDGLAPPQHLIRVEGNLRVEYLDDRNTFRHSV |
| | | VVPYEPPEVGSDCTTIHYNYMCNSSCMGGMNRRPILTIITLEDS |
| | | SGNLLGRNSFEVRVCACPGRDRRTEEENLRKKGEPHHELPPGS |
| | | TKRALPNNTSSSPQPKKKPLDGEYFTLQMLLDLRWCYFLINSS |

Compositions and methods disclosed herein include human p53 knock-in (Hupki) mice and cancer cell lines derived therefrom that can be used for studying cancer etiology and for testing potential cancer therapeutic agents.

### Engineered Non-Human Animals and Cells

Provided herein are engineered non-human animals and cells that encode a p53 protein of the disclosure, for example, a p53 protein with a sequence from human p53 that comprises an amino acid substitution at position 220 relative to human p53.

### Human p53 Knock-in (HUPKI) Non-human Animal

Hupki mice are a biological tool for studying cancer therapeutics and etiology. In Hupki mice, parts of the endogenous mouse p53 allele (e.g., exons 4-9) can be replaced with the homologous human p53 gene sequence. A wild type Hupki allele can function normally, and the Hupki protein can bind p53 consensus sequences and respond to various stimuli. Wild type Hupki mice allow researchers to examine in vivo spontaneous and induced mutations in human p53 gene sequences, and test pharmaceuticals designed to modulate DNA-binding activity of human p53.

Hupki p53 mutant mice can be generated with relevant hot-spot mutations incorporated into the human p53 gene sequence (e.g., R248Q and G245S), and the resulting mice and cells derived therefrom can be used for studying cancer etiology and for testing potential cancer therapeutic agents and combination therapies.

Disclosed herein, in some embodiments, are Hupki-p53 mice with a Y220 mutation in p53. In some embodiments, the Hupki-p53 mice have a Y220C mutation in p53. In some embodiments, the Hupki-p53 mice have a Y220S mutation in p53. In some embodiments, the Hupki-p53 mice have a Y220H mutation in p53.

In some embodiments, the Hupki-p53 mice have a Y220C mutation in p53. In some embodiments, Hupki-Y220C mice disclosed herein are engineered to harbor exons 4-9 of human p53 and flanking mouse exons (e.g., 1-2 and 10-11), with the Y220C mutation in exon 6. These Hupki-Y220C mice can be used, for example, to examine tumorigenesis in different tissues, investigate mechanisms of gain of function, study downstream genetic and signal transduction pathways, generate cancer cell lines that can be implanted into recipient mice, and test potential therapeutics.

A non-human animal of the disclosure can comprise parts of a human p53 gene sequence, for example, parts of the endogenous p53 allele (e.g., exons 4-9) can be replaced with the homologous human p53 gene sequence. The non-human animal can be generated with relevant hot-spot mutations incorporated into the human p53 gene sequence, and the resulting engineered non-human animal and cells derived therefrom can be used for studying cancer etiology and for testing potential cancer therapeutic agents and combination therapies.

Disclosed herein, in some embodiments, are non-human animals that comprise parts of a human p53 gene sequence with a Y220 mutation relative to human p53. In some embodiments, the non-human animals have a Y220C mutation relative to human p53. In some embodiments, the non-human animals have a Y220S mutation relative to human p53. In some embodiments, the non-human animals have a Y220H mutation relative to human p53.

In some embodiments, non-human animals disclosed herein are engineered to harbor one or more exons (e.g., exons 4-9) of human p53 and flanking exons from p53 that are endogenous to the non-human animal (e.g., exons 1-2 and 10-11), with the Y220C mutation in exon 6. These non-human animals can be used, for example, to examine tumorigenesis in different tissues, investigate mechanisms of gain of function, study downstream genetic and signal transduction pathways, generate cancer cell lines that can be implanted into recipient mice, and test potential therapeutics.

In some embodiments, a non-human animal is a mammal. In some embodiments, a non-human animal is a rodent. In some embodiments, a non-human animal is a mouse. In some embodiments, a non-human animal is a rat. In some embodiments, a non-human animal is a rabbit. In some embodiments, a non-human animal is a guinea pig. In some embodiments, a non-human animal is a hamster. In some embodiments, a non-human animal is a pig.

In some embodiments, a non-human animal is an inbred mouse strain. In some embodiments, a non-human animal is a mouse with a C57BL/6 genetic background. In some embodiments, a non-human animal is an inbred mouse strain. In some embodiments, a non-human animal is a mouse with a C57BL/6, BALB/C, 129Sv, C3H, DBA/2J, A/J, or FVB/N genetic background, or a combination thereof In some embodiments, a non-human animal is a mouse with a 129Sv/C57BL6 mixed background. In some embodiments, a non-human animal is a mouse with a 129Sv/C57BL6 mixed genetic background.

In some embodiments, a non-human animal is an immunocompetent animal. In some embodiments, a non-human animal is an immunodeficient animal.

In some embodiments, the disclosure provides engineered cells, for example, comprising a nucleic acid that encodes a p53 protein of the disclosure.

In some embodiments, the engineered cells are non-human mammalian cells derived from non-human animals disclosed herein. In some embodiments, the engineered cells are not derived from a non-human animals disclosed herein, for example, the cells can be generated in vitro or in vivo.

In some embodiments, the engineered cells are cell lines. In some embodiments, the engineered cells are primary cells. In some embodiments, the engineered cells are cancer cells. In some embodiments, the engineered cells are metastatic cancer cells. In some embodiments, the engineered cells are cells with pre-cancerous mutations.

In some embodiments, the engineered cells are harvested from spontaneous or induced tumors in a non-human animal of the disclosure. Such engineered cells can be used in cancer models of the disclosure, for example to study the anti-cancer activity of a candidate therapeutic agent in vitro. In some embodiments, the engineered cells can be used in an in vivo cancer model, for example, the cells can be administered to syngeneic animals, in which they proliferate to form a cancer that harbors the p53 nucleic acid or protein of the disclosure.

In some embodiments, the engineered cells are harvested from a cancer in a non-human animal of the disclosure, for example, an acute leukemia, astrocytoma, biliary cancer (cholangiocarcinoma), bone cancer, breast cancer, brain stem glioma, bronchioloalveolar cell lung cancer, cancer of the adrenal gland, cancer of the anal region, cancer of the bladder, cancer of the endocrine system, cancer of the esophagus, cancer of the head or neck, cancer of the kidney, cancer of the parathyroid gland, cancer of the penis, cancer of the pleural/peritoneal membranes, cancer of the salivary gland, cancer of the small intestine, cancer of the thyroid gland, cancer of the ureter, cancer of the urethra, carcinoma of the cervix, carcinoma of the endometrium, carcinoma of the fallopian tubes, carcinoma of the renal pelvis, carcinoma of the vagina, carcinoma of the vulva, cervical cancer, chronic leukemia, colon cancer, colorectal cancer, cutaneous melanoma, ependymoma, epidermoid tumors, Ewings sarcoma, gastric cancer, glioblastoma, glioblastoma multiforme, glioma, hematologic malignancies, hepatocellular (liver) carcinoma, hepatoma, Hodgkin's Disease, intraocular melanoma, Kaposi sarcoma, lung cancer, lymphomas, medulloblastoma, melanoma, meningioma, mesothelioma, multiple myeloma, muscle cancer, neoplasms of the central nervous system (CNS), neuronal cancer, small cell lung cancer, non-small cell lung cancer, osteosarcoma, ovarian cancer, pancreatic cancer, pediatric malignancies, pituitary adenoma, prostate cancer, rectal cancer, renal cell carcinoma, sarcoma of soft tissue, schwanoma, skin cancer, spinal axis tumors, squamous cell carcinomas, stomach cancer, synovial sarcoma, testicular cancer, uterine cancer, or tumors and their metastases, including refractory versions of any of the above cancers, or a combination thereof.

Engineered non-human animals and cells of the disclosure can be generated using any suitable techniques, for example, methods that include nuclease gene editing tools, homologous recombination, non-homologous recombination, homology-directed repair, transposon systems, somatic cell nuclear transfer, or any combination thereof. An engineered non-human animal or cell can be a transchromosomal, for example, can comprise an artificial chromosome, for example, a bacterial artificial chromosome or yeast artificial chromosome. In some embodiments, an engineered non-human animal or cell is transgenic. In some embodiments, an engineered non-human animal or cell is a knock-in animal or cell.

A variety of enzymes can catalyze insertion of foreign DNA into a host genome. Non-limiting examples of gene editing tools and techniques include CRISPR, TALEN, zinc finger nuclease (ZFN), meganuclease, Mega-TAL, and transposon-based systems.

A CRISPR system can be utilized to facilitate insertion of a nucleotide sequence into a cell genome. For example, a CRISPR system can introduce a double stranded break at a target site in a genome. There are at least five types of CRISPR systems which all incorporate RNAs and CRISPR-associated proteins (Cas). Types I, III, and IV assemble a multi-Cas protein complex that is capable of cleaving nucleic acids that are complementary to the crRNA. Types I and III both require pre-crRNA processing prior to assembling the processed crRNA into the multi-Cas protein complex. Types II and V CRISPR systems comprise a single Cas protein complexed with at least one guiding RNA. A transposon based system can be utilized for insertion of a nucleic acid into a genome.

Constructs used in generation of non-human animals and cells of the disclosure can be introduced using any suitable method, including but not limited to electroporation, viral vectors, liposomes, microparticles, nanoparticles, dendrimers, lentiviral transduction, sonoporation, use of a gene gun, lipofection, calcium phosphate transfection, and/or microinjection. Viral vector delivery systems can include DNA and RNA viruses, which have either episomal or integrated genomes after delivery to the cell. Examples of viral vectors include, but are not limited to, retroviral vectors, lentiviral vectors, adenovirus vectors, poxvirus vectors; herpesvirus vectors and adeno-associated virus (AAV) vectors, helper-dependent adenovirus vectors, hybrid adenovirus vectors, Epstein-Bar virus vectors, herpes simplex virus vectors, hemagglutinating virus of Japan (HVJ) vectors, and Moloney murine leukemia virus vectors. In some embodiments, the constructs are introduced into embryonic stem cells. In some embodiments, the constructs are introduced into cells that are not embryonic stem cells.

In some embodiments, drug selection or reporter gene selection can be used to select or enrich engineered cells that incorporate a construct.

In some embodiments, embryos comprising a nucleic acid sequence of the disclosure (e.g., that encodes a p53 protein) are introduced into surrogate mothers. Any suitable breeding techniques can be used to generate non-human animals that are heterozygous or homozygous for the nucleic acid sequence of the disclosure, for example, genotyping parental mice, mating F0 animals to appropriate partners to obtain F1 animals, mating F1 animals to obtain F2 animals, backcrossing, etc.

### p53 Proteins and Nucleic Acids

In some embodiments, a p53 protein of the disclosure comprises an amino acid sequence with at least about 70%, at least about 75%, at least about 80%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 98.5%, at least about 99%, at least about 99.5%, or about 100% sequence identity or sequence similarity to SEQ ID NO: 1. The p53 protein can comprise an amino acid substitution at position 220 relative to SEQ ID NO: 1. In some embodiments, the p53 protein includes arginine instead of proline at position 72 relative to SEQ ID NO: 1 (P72R).

In some embodiments, a p53 protein of the disclosure comprises an amino acid sequence with at most about 70%, at most about 75%, at most about 80%, at most about 85%, at most about 86%, at most about 87%, at most about 88%, at most about 89%, at most about 90%, at most about 91%, at most about 92%, at most about 93%, at most about 94%, at most about 95%, at most about 96%, at most about 97%, at most about 98%, at most about 98.5%, at most about 99%, at most about 99.5%, or about 100% sequence identity or sequence similarity to SEQ ID NO: 1. The p53 protein can comprise an amino acid substitution at position 220 relative to SEQ ID NO: 1. In some embodiments, the p53 protein includes arginine instead of proline at position 72 relative to SEQ ID NO: 1 (P72R).

In some embodiments, a p53 protein of the disclosure comprises an amino acid sequence with about 70%, about 75%, about 80%, about 85%, about 86%, about 87%, about 88%, about 89%, about 90%, about 91%, about 92%, about 93%, about 94%, about 95%, about 96%, about 97%, about 98%, about 98.5%, about 99%, about 99.5%, or about 100% sequence identity or sequence similarity to SEQ ID NO: 1. The p53 protein can comprise an amino acid substitution at position 220 relative to SEQ ID NO: 1. In some embodiments, the p53 protein includes arginine instead of proline at position 72 relative to SEQ ID NO: 1 (P72R).

In some embodiments, a p53 protein of the disclosure comprises the amino acid sequence of SEQ ID NO: 1. In some embodiments, a p53 protein of the disclosure consists essentially of the amino acid sequence of SEQ ID NO: 1. In some embodiments, a p53 protein of the disclosure consists of the amino acid sequence of SEQ ID NO: 1.

In some embodiments, a p53 protein of the disclosure is a fusion protein that comprises one or more amino acid sequences from SEQ ID NO: 1 and one or more amino acid sequences from a different p53 protein, for example, from a different species (e.g., a non-human mammal, such as a mouse). In some embodiments, a p53 protein of the disclosure is a fusion protein that comprises one or more amino acid sequences that are encoded by a TP53 gene that codes for SEQ ID NO: 1, and one or more amino acid sequences that are encoded by a different TP53 gene, for example, from a different species (e.g., a non-human mammal, such as a mouse). The p53 protein can comprise an amino acid substitution at position 220 relative to SEQ ID NO: 1. In some embodiments, the p53 protein includes arginine instead of proline at position 72 relative to SEQ ID NO: 1 (P72R).

In some embodiments, a p53 protein of the disclosure is a fusion protein that comprises one or more amino acid sequences that are encoded by one or more exons of a TP53 gene that codes for SEQ ID NO: 1, and one or more amino acid sequences that are encoded by one or more exons of a different TP53 gene, for example, from a different species (e.g., a non-human mammal, such as a mouse). The p53 protein can comprise an amino acid substitution at position 220 relative to SEQ ID NO: 1. In some embodiments, the p53 protein includes arginine instead of proline at position 72 relative to SEQ ID NO: 1 (P72R).

In some embodiments, a p53 protein of the disclosure is a fusion protein that comprises an amino acid sequence with at least about 70%, at least about 75%, at least about 80%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 98.5%, at least about 99%, at least about 99.5%, or about 100% sequence identity to an amino acid sequence encoded by exon 6 of a TP53 gene that codes for SEQ ID NO: 1. The p53 protein can comprise an amino acid substitution at position 220 relative to SEQ ID NO: 1. In some embodiments, the p53 protein includes arginine instead of proline at position 72 relative to SEQ ID NO: 1 (P72R). In some embodiments, an amino acid sequence encoded by exon 6 of the TP53 gene is SEQ ID NO: 25. In some embodiments, an amino acid sequence encoded by exon 6 of the TP53 gene is SEQ ID NO: 46.

In some embodiments, a p53 protein of the disclosure is a fusion protein that comprises an amino acid sequence with at least 90% sequence identity to an amino acid sequence encoded by exon 6 of a TP53 gene that codes for SEQ ID NO: 1. The p53 protein can comprise an amino acid substitution at position 220 relative to SEQ ID NO: 1. In some embodiments, the p53 protein includes arginine instead of proline at position 72 relative to SEQ ID NO: 1 (P72R). In some embodiments, an amino acid sequence encoded by exon 6 of the TP53 gene is SEQ ID NO: 25. In some embodiments, an amino acid sequence encoded by exon 6 of the TP53 gene is SEQ ID NO: 46.

In some embodiments, a p53 protein of the disclosure is a fusion protein that comprises an amino acid sequence with at least about 70%, at least about 75%, at least about 80%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 98.5%, at least about 99%, at least about 99.5%, or about 100% sequence identity to an amino acid sequence encoded by exon 1, exon 2, exon 3, exon 4, exon 5, exon 6, exon 7, exon 8, exon 9 exon 10, or exon 11 of a TP53 gene that codes for SEQ ID NO: 1. The p53 protein can comprise an amino acid substitution at position 220 relative to SEQ ID NO: 1. In some embodiments, the p53 protein includes arginine instead of proline at position 72 relative to SEQ ID NO: 1 (P72R).

In some embodiments, a p53 protein of the disclosure is a fusion protein that comprises amino acid sequences with at least about 70%, at least about 75%, at least about 80%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 98.5%, at least about 99%, at least about 99.5%, or about 100% sequence identity to amino acid sequences encoded by exon 5, exon 6, and/or exon 7 of a TP53 gene that codes for SEQ ID NO: 1. The p53 protein can comprise an amino acid substitution at position 220 relative to SEQ ID NO: 1. In some embodiments, the p53 protein includes arginine instead of proline at position 72 relative to SEQ ID NO: 1 (P72R).

In some embodiments, the amino acid sequence encoded by exon 2 comprises an amino acid sequence of SEQ ID NO: 17. In some embodiments, the amino acid sequence encoded by exon 3 comprises an amino acid sequence of SEQ ID NO: 19. In some embodiments, the amino acid sequence encoded by exon 4 comprises an amino acid sequence of SEQ ID NO: 21. In some embodiments, the amino acid sequence encoded by exon 5 comprises an amino acid sequence of SEQ ID NO: 23. In some embodiments, the amino acid sequence encoded by exon 6 comprises an amino acid sequence of SEQ ID NO: 25. In some embodiments, the amino acid sequence encoded by exon 7 comprises an amino acid sequence of SEQ ID NO: 27. In some embodiments, the amino acid sequence encoded by exon 8 comprises an amino acid sequence of SEQ ID NO: 29. In some embodiments, the amino acid sequence encoded by exon 9 comprises an amino acid sequence of SEQ ID NO: 31. In some embodiments, the amino acid sequence encoded by exon 10 comprises an amino acid sequence of SEQ ID NO: 33. In some embodiments, the amino acid sequence encoded by exon 11 comprises an amino acid sequence of SEQ ID NO: 35.

In some embodiments, a p53 protein of the disclosure is a fusion protein that comprises amino acid sequences with at least about 70%, at least about 75%, at least about 80%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 98.5%, at least about 99%, at least about 99.5%, or about 100% sequence identity to amino acid sequences encoded by exon 4, exon 5, exon 6, exon 7, exon 8, and exon 9 of a TP53 gene that codes for SEQ ID NO: 1. The p53 protein can comprise an amino acid substitution at position 220 relative to SEQ ID NO: 1. In some embodiments, the p53 protein includes arginine instead of proline at position 72 relative to SEQ ID NO: 1 (P72R).

In some embodiments, a p53 protein of the disclosure is a fusion protein that comprises amino acid sequences with at least about 90% sequence identity to amino acid sequences encoded by exon 4, exon 5, exon 6, exon 7, exon 8, and exon 9 of a TP53 gene that codes for SEQ ID NO: 1, and the p53 protein comprises an amino acid substitution at position 220 relative to SEQ ID NO: 1. In some embodiments, the p53 protein includes arginine instead of proline at position 72 relative to SEQ ID NO: 1 (P72R).

In some embodiments, the amino acid sequence encoded by exon 4 comprises an amino acid sequence of SEQ ID NO: 21. In some embodiments, the amino acid sequence encoded by exon 5 comprises an amino acid sequence of SEQ ID NO: 23. In some embodiments, the amino acid sequence encoded by exon 6 comprises an amino acid sequence of SEQ ID NO: 25. In some embodiments, the amino acid sequence encoded by exon 7 comprises an amino acid sequence of SEQ ID NO: 27. In some embodiments, the amino acid sequence encoded by exon 8 comprises an amino acid sequence of SEQ ID NO: 29. In some embodiments, the amino acid sequence encoded by exon 9 comprises an amino acid sequence of SEQ ID NO: 31.

The disclosure also provides nucleic acids that encode p53 proteins. For example, in some embodiments, the disclosure provides a nucleic acid that encodes an amino acid sequence with at least about 70%, at least about 75%, at least about 80%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 98.5%, at least about 99%, at least about 99.5%, or about 100% sequence identity or sequence similarity to SEQ ID NO: 1. In some embodiments, the nucleic acid encodes a p53 protein that includes arginine instead of proline at position 72 relative to SEQ ID NO: 1 (P72R).

In some embodiments, a nucleic acid encodes an amino acid sequence with at most about 70%, at most about 75%, at most about 80%, at most about 85%, at most about 86%, at most about 87%, at most about 88%, at most about 89%, at most about 90%, at most about 91%, at most about 92%, at most about 93%, at most about 94%, at most about 95%, at most about 96%, at most about 97%, at most about 98%, at most about 98.5%, at most about 99%, at most about 99.5%, or about 100% sequence identity or sequence similarity to SEQ ID NO: 1. In some embodiments, the nucleic acid encodes a p53 protein that includes arginine instead of proline at position 72 relative to SEQ ID NO: 1 (P72R).

In some embodiments, a nucleic acid encodes an amino acid sequence with about 70%, about 75%, about 80%, about 85%, about 86%, about 87%, about 88%, about 89%, about 90%, about 91%, about 92%, about 93%, about 94%, about 95%, about 96%, about 97%, about 98%, about 98.5%, about 99%, about 99.5%, or about 100% sequence identity or sequence similarity to SEQ ID NO: 1. In some embodiments, the nucleic acid encodes a p53 protein that includes arginine instead of proline at position 72 relative to SEQ ID NO: 1 (P72R).

In some embodiments, a nucleic acid encodes the amino acid sequence of SEQ ID NO: 1. In some embodiments, a nucleic acid encodes an amino acid sequence that consists essentially of the amino acid sequence of SEQ ID NO: 1. In some embodiments, a nucleic acid encodes an amino acid sequence that consists of the amino acid sequence of SEQ ID NO: 1. In some embodiments, the nucleic acid encodes a p53 protein that includes arginine instead of proline at position 72 relative to SEQ ID NO: 1 (P72R).

In some embodiments, a nucleic acid encodes a fusion protein that comprises one or more amino acid sequences from SEQ ID NO: 1 and one or more amino acid sequences from a different p53 protein, for example, from a different species (e.g., a non-human mammal, such as a mouse).

In some embodiments, a nucleic acid comprises one or more exons of a TP53 gene that codes for SEQ ID NO: 1, and one or more exons of different TP53 gene, for example, from a different species (e.g., a non-human mammal, such as a mouse).

In some embodiments, a nucleic acid comprises a sequence with at least about 70%, at least about 75%, at least about 80%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 98.5%, at least about 99%, at least about 99.5%, or about 100% sequence identity to exon 6 of a TP53 gene that codes for SEQ ID NO: 1. The nucleic acid can encode an amino acid substitution at position 220 relative to SEQ ID NO: 1. In some embodiments, the nucleic acid encodes a p53 protein that includes arginine instead of proline at position 72 relative to SEQ ID NO: 1 (P72R).

In some embodiments, a nucleic acid comprises a sequence with at least about 90% sequence identity to exon 6 of a TP53 gene that codes for SEQ ID NO: 1, the nucleic acid encodes an amino acid substitution at position 220 relative to SEQ ID NO: 1, and the nucleic acid encodes arginine instead of proline at position 72 relative to SEQ ID NO: 1 (P72R).

In some embodiments, a nucleic acid comprises a sequence with at least about 70%, at least about 75%, at least about 80%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 98.5%, at least about 99%, at least about 99.5%, or about 100% sequence identity to exon 1, exon 2, exon 3, exon 4, exon 5, exon 6, exon 7, exon 8, exon 9 exon 10, or exon 11 of a TP53 gene that codes for SEQ ID NO: 1. The nucleic acid can encode an amino acid substitution at position 220 relative to SEQ ID NO: 1. In some embodiments, the nucleic acid encodes arginine instead of proline at position 72 relative to SEQ ID NO: 1 (P72R).

In some embodiments, the nucleic acid sequence encoded by exon 2 comprises a nucleic acid sequence of SEQ ID NO: 16. In some embodiments, the nucleic acid sequence encoded by exon 3 comprises a nucleic acid sequence of SEQ ID NO: 18. In some embodiments, the nucleic acid sequence encoded by exon 4 comprises a nucleic acid sequence of SEQ ID NO: 20. In some embodiments, the nucleic acid sequence encoded by exon 5 comprises a nucleic acid sequence of SEQ ID NO: 22. In some embodiments, the nucleic acid sequence encoded by exon 6 comprises a nucleic acid sequence of SEQ ID NO: 24. In some embodiments, the nucleic acid sequence encoded by exon 7 comprises a nucleic acid sequence of SEQ ID NO: 26. In some embodiments, the nucleic acid sequence encoded by exon 8 comprises a nucleic acid sequence of SEQ ID NO: 28. In some embodiments, the nucleic acid sequence encoded by exon 9 comprises a nucleic acid sequence of SEQ ID NO: 30. In some embodiments, the nucleic acid sequence encoded by exon 10 comprises a nucleic acid sequence of SEQ ID NO: 32. In some embodiments, the nucleic acid sequence encoded by exon 11 comprises a nucleic acid sequence of SEQ ID NO: 34.

In some embodiments, a nucleic acid comprises a sequence with at least about 70%, at least about 75%, at least about 80%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 98.5%, at least about 99%, at least about 99.5%, or about 100% sequence identity to exon 5, exon 6, and/or exon 7 of a TP53 gene that codes for SEQ ID NO: 1. The nucleic acid can encode an amino acid substitution at position 220 relative to SEQ ID NO: 1. In some embodiments, the nucleic acid encodes arginine instead of proline at position 72 relative to SEQ ID NO: 1 (P72R).

In some embodiments, a nucleic acid comprises a sequence with at least about 70%, at least about 75%, at least about 80%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 98.5%, at least about 99%, at least about 99.5%, or about 100% sequence identity to exon 4, exon 5, exon 6, exon 7, exon 8, and/or exon 9 of a TP53 gene that codes for SEQ ID NO: 1. The nucleic acid can encode an amino acid substitution at position 220 relative to SEQ ID NO: 1. In some embodiments, the nucleic acid encodes arginine instead of proline at position 72 relative to SEQ ID NO: 1 (P72R).

In some embodiments, a nucleic acid comprises sequences with at least about 90% sequence identity to each of exon 4, exon 5, exon 6, exon 7, exon 8, and exon 9 of a TP53 gene that codes for SEQ ID NO: 1, and the nucleic acid encodes an amino acid substitution at position 220 relative to SEQ ID NO: 1. In some embodiments, the nucleic acid encodes arginine instead of proline at position 72 relative to SEQ ID NO: 1 (P72R).

In some embodiments, the nucleic acid sequence encoded by exon 4 comprises a nucleic acid sequence of SEQ ID NO: 20. In some embodiments, the nucleic acid sequence encoded by exon 5 comprises a nucleic acid sequence of SEQ ID NO: 22. In some embodiments, the nucleic acid sequence encoded by exon 6 comprises a nucleic acid sequence of SEQ ID NO: 24. In some embodiments, the nucleic acid sequence encoded by exon 7 comprises a nucleic acid sequence of SEQ ID NO: 26. In some embodiments, the nucleic acid sequence encoded by exon 8 comprises a nucleic acid sequence of SEQ ID NO: 28. In some embodiments, the nucleic acid sequence encoded by exon 9 comprises a nucleic acid sequence of SEQ ID NO: 30.

In some embodiments, a p53 protein of the disclosure comprises an amino acid sequence with at least about 70%, at least about 75%, at least about 80%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 98.5%, at least about 99%, at least about 99.5%, or about 100% sequence identity or sequence similarity to any one of SEQ ID NOS: 2-9.

In some embodiments, a p53 protein of the disclosure comprises an amino acid sequence with at most about 70%, at most about 75%, at most about 80%, at most about 85%, at most about 86%, at most about 87%, at most about 88%, at most about 89%, at most about 90%, at most about 91%, at most about 92%, at most about 93%, at most about 94%, at most about 95%, at most about 96%, at most about 97%, at most about 98%, at most about 98.5%, at most about 99%, at most about 99.5%, or about 100% sequence identity or sequence similarity to any one of SEQ ID NOS: 2-9.

In some embodiments, a p53 protein of the disclosure comprises an amino acid sequence with about 70%, about 75%, about 80%, about 85%, about 86%, about 87%, about 88%, about 89%, about 90%, about 91%, about 92%, about 93%, about 94%, about 95%, about 96%, about 97%, about 98%, about 98.5%, about 99%, about 99.5%, or about 100% sequence identity or sequence similarity to any one of SEQ ID NOs: 2-9.

In some embodiments, a p53 protein of the disclosure comprises the amino acid sequence of any one of SEQ ID NOs: 2-9. In some embodiments, a p53 protein of the disclosure consists essentially of the amino acid sequence of any one of SEQ ID NOs: 2-9. In some embodiments, a p53 protein of the disclosure consists of the amino acid sequence of any one of SEQ ID NOs: 2-9.

In some embodiments, a p53 protein of the disclosure is a fusion protein that comprises one or more amino acid sequences from any one of SEQ ID NOs: 2-9 and one or more amino acid sequences from a different p53 protein, for example, from a different species (e.g., a non-human mammal, such as a mouse). In some embodiments, a p53 protein of the disclosure is a fusion protein that comprises one or more amino acid sequences that are encoded by a TP53 gene that codes for any one of SEQ ID NOs: 2-9, and one or more amino acid sequences that are encoded by a different TP53 gene, for example, from a different species (e.g., a non-human mammal, such as a mouse).

In some embodiments, a p53 protein of the disclosure is a fusion protein that comprises one or more amino acid sequences that are encoded by one or more exons of a TP53 gene that codes for any one of SEQ ID NOs: 2-9, and one or more amino acid sequences that are encoded by one or more exons of a different TP53 gene, for example, from a different species (e.g., a non-human mammal, such as a mouse).

In some embodiments, a p53 protein of the disclosure is a fusion protein that comprises an amino acid sequence with at least about 70%, at least about 75%, at least about 80%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 98.5%, at least about 99%, at least about 99.5%, or about 100% sequence identity to an amino acid sequence encoded by exon 6 of a TP53 gene that codes for any one of SEQ ID NOs: 2-9. The p53 protein can comprise an amino acid substitution at position 220 relative SEQ ID NO: 1.

In some embodiments, a p53 protein of the disclosure is a fusion protein that comprises an amino acid sequence with at least about 70%, at least about 75%, at least about 80%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 98.5%, at least about 99%, at least about 99.5%, or about 100% sequence identity to an amino acid sequence encoded by exon 1, exon 2, exon 3, exon 4, exon 5, exon 6, exon 7, exon 8, exon 9 exon 10, or exon 11 of a TP53 gene that codes for any one of SEQ ID NOs: 2-9. The p53 protein can comprise an amino acid substitution at position 220 relative SEQ ID NO: 1.

In some embodiments, a p53 protein of the disclosure is a fusion protein that comprises amino acid sequences with at least about 70%, at least about 75%, at least about 80%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 98.5%, at least about 99%, at least about 99.5%, or about 100% sequence identity to amino acid sequences encoded by exon 5, exon 6, and exon 7 of a TP53 gene that codes for any one of SEQ ID NOs: 2-9. The p53 protein can comprise an amino acid substitution at position 220 relative to SEQ ID NO: 1.

In some embodiments, a p53 protein of the disclosure is a fusion protein that comprises amino acid sequences with at least about 70%, at least about 75%, at least about 80%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 98.5%, at least about 99%, at least about 99.5%, or about 100% sequence identity to amino acid sequences encoded by exon 4, exon 5, exon 6, exon 7, exon 8, and exon 9 of a TP53 gene that codes for any one of SEQ ID NOs: 2-9. The p53 protein can comprise an amino acid substitution at position 220 relative SEQ ID NO: 1.

In some embodiments, the amino acid sequence encoded by exon 4 comprises an amino acid sequence of SEQ ID NO: 21. In some embodiments, the amino acid sequence encoded by exon 5 comprises an amino acid sequence of SEQ ID NO: 23. In some embodiments, the amino acid sequence encoded by exon 6 comprises an amino acid sequence of SEQ ID NO: 25. In some embodiments, the amino acid sequence encoded by exon 7 comprises an amino acid sequence of SEQ ID NO: 27. In some embodiments, the amino acid sequence encoded by exon 8 comprises an amino acid sequence of SEQ ID NO: 29. In some embodiments, the amino acid sequence encoded by exon 9 comprises an amino acid sequence of SEQ ID NO: 31.

In some embodiments, the disclosure provides a nucleic acid that encodes an amino acid sequence with at least about 70%, at least about 75%, at least about 80%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 98.5%, at least about 99%, at least about 99.5%, or about 100% sequence identity or sequence similarity to any one of SEQ ID NOs: 2-9.

In some embodiments, a nucleic acid encodes an amino acid sequence with at most about 70%, at most about 75%, at most about 80%, at most about 85%, at most about 86%, at most about 87%, at most about 88%, at most about 89%, at most about 90%, at most about 91%, at most about 92%, at most about 93%, at most about 94%, at most about 95%, at most about 96%, at most about 97%, at most about 98%, at most about 98.5%, at most about 99%, at most about 99.5%, or about 100% sequence identity or sequence similarity to any one of SEQ ID NOs: 2-9.

In some embodiments, a nucleic acid encodes an amino acid sequence with about 70%, about 75%, about 80%, about 85%, about 86%, about 87%, about 88%, about 89%, about 90%, about 91%, about 92%, about 93%, about 94%, about 95%, about 96%, about 97%, about 98%, about 98.5%, about 99%, about 99.5%, or about 100% sequence identity or sequence similarity to any one of SEQ ID NOs: 2-9.

In some embodiments, a nucleic acid encodes the amino acid sequence of any one of SEQ ID NOs: 2-9. In some embodiments, a nucleic acid encodes an amino acid sequence that consists essentially of the amino acid sequence of any one of SEQ ID NOs: 2-9. In some embodiments, a nucleic acid encodes an amino acid sequence that consists of the amino acid sequence of any one of SEQ ID NOs: 2-9.

In some embodiments, a nucleic acid encodes a fusion protein that comprises one or more amino acid sequences from any one of SEQ ID NOs: 2-9 and one or more amino acid sequences from a different p53 protein, for example, from a different species (e.g., a non-human mammal, such as a mouse).

In some embodiments, a nucleic acid comprises one or more exons of a TP53 gene that codes for any one of SEQ ID NOs: 2-9, and one or more exons of different TP53 gene, for example, from a different species (e.g., a non-human mammal, such as a mouse).

In some embodiments, a nucleic acid comprises a sequence with at least about 70%, at least about 75%, at least about 80%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 98.5%, at least about 99%, at least about 99.5%, or about 100% sequence identity to exon 6 of a TP53 gene that codes for any one of SEQ ID NOs: 2-9. The nucleic acid can encode an amino acid substitution at position 220 relative to any one of SEQ ID NO: 1.

In some embodiments, a nucleic acid comprises a sequence with at least about 70%, at least about 75%, at least about 80%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 98.5%, at least about 99%, at least about 99.5%, or about 100% sequence identity to exon 1, exon 2, exon 3, exon 4, exon 5, exon 6, exon 7, exon 8, exon 9 exon 10, or exon 11 of a TP53 gene that codes for any one of SEQ ID NOs: 2-9. The nucleic acid can encode an amino acid substitution at position 220 relative to any one of SEQ ID NO: 1.

In some embodiments, the nucleic acid sequence encoded by exon 2 comprises a nucleic acid sequence of SEQ ID NO: 16. In some embodiments, the nucleic acid sequence encoded by exon 3 comprises a nucleic acid sequence of SEQ ID NO: 18. In some embodiments, the nucleic acid sequence encoded by exon 4 comprises a nucleic acid sequence of SEQ ID NO: 20. In some embodiments, the nucleic acid sequence encoded by exon 5 comprises a nucleic acid sequence of SEQ ID NO: 22. In some embodiments, the nucleic acid sequence encoded by exon 6 comprises a nucleic acid sequence of SEQ ID NO: 24. In some embodiments, the nucleic acid sequence encoded by exon 7 comprises a nucleic acid sequence of SEQ ID NO: 26. In some embodiments, the nucleic acid sequence encoded by exon 8 comprises a nucleic acid sequence of SEQ ID NO: 28. In some embodiments, the nucleic acid sequence encoded by exon 9 comprises a nucleic acid sequence of SEQ ID NO: 30. In some embodiments, the nucleic acid sequence encoded by exon 10 comprises a nucleic acid sequence of SEQ ID NO: 32. In some embodiments, the nucleic acid sequence encoded by exon 11 comprises a nucleic acid sequence of SEQ ID NO: 34.

In some embodiments, a nucleic acid comprises a sequence with at least about 70%, at least about 75%, at least about 80%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 98.5%, at least about 99%, at least about 99.5%, or about 100% sequence identity to exon 5, exon 6, and/or exon 7 of a TP53 gene that codes for any one of SEQ ID NOs: 2-9. The nucleic acid can encode an amino acid substitution at position 220 relative to any one of SEQ ID NO: 1.

In some embodiments, a nucleic acid comprises a sequence with at least about 70%, at least about 75%, at least about 80%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 98.5%, at least about 99%, at least about 99.5%, or about 100% sequence identity to exon 4, exon 5, exon 6, exon 7, exon 8, and/or exon 9 of a TP53 gene that codes for any one of SEQ ID NOs: 2-9. The nucleic acid can encode an amino acid substitution at position 220 relative to any one of SEQ ID NO: 1.

In some embodiments, a p53 protein of the disclosure is a fusion protein that comprises one or more amino acid sequences that are encoded by one or more exons (e.g., exon 1, 2, 3, 4, 5, 6, 7, 8, 9, 10, and/or 11) of a TP53 gene from a non-human mammal, for example, a mouse. In some embodiments, a p53 protein of the disclosure comprises an amino acid sequence encoded by exon 1 of a TP53 gene from a non-human mammal, for example, a mouse. In some embodiments, a p53 protein of the disclosure comprises an amino acid sequence encoded by exon 2 of a TP53 gene from a non-human mammal, for example, a mouse. In some embodiments, a p53 protein of the disclosure comprises an amino acid sequence encoded by exon 3 of a TP53 gene from a non-human mammal, for example, a mouse. In some embodiments, a p53 protein of the disclosure comprises an amino acid sequence encoded by exon 10 of a TP53 gene from a non-human mammal, for example, a mouse. In some embodiments, a p53 protein of the disclosure comprises an amino acid sequence encoded by exon 11 of a TP53 gene from a non-human mammal, for example, a mouse. In some embodiments, the amino acid sequences contain at least about 70%, at least about 75%, at least about 80%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 98.5%, at least about 99%, at least about 99.5%, or about 100% sequence identity to the corresponding sequences in a wild type p53 protein from the non-human mammal.

In some embodiments, a nucleic acid that encodes a p53 protein of the disclosure comprises one or more exons (e.g., exon 1, 2, 3, 4, 5, 6, 7, 8, 9, 10, and/or 11) of a TP53 gene from a non-human mammal, for example, a mouse. In some embodiments, a nucleic acid that encodes a p53 protein of the disclosure comprises exon 1 of a TP53 gene from a non-human mammal, for example, a mouse. In some embodiments, a nucleic acid that encodes a p53 protein of the disclosure comprises exon 2 of a TP53 gene from a non-human mammal, for example, a mouse. In some embodiments, a nucleic acid that encodes a p53 protein of the disclosure comprises exon 3 of a TP53 gene from a non-human mammal, for example, a mouse. In some embodiments, a nucleic acid that encodes a p53 protein of the disclosure comprises exon 10 of a TP53 gene from a non-human mammal, for example, a mouse. In some embodiments, a nucleic acid that encodes a p53 protein of the disclosure comprises exon 11 of a TP53 gene from a non-human mammal, for example, a mouse. In some embodiments, the nucleic acid contain at least about at least about 70%, at least about 75%, at least about 80%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 98.5%, at least about 99%, at least about 99.5%, or about 100% sequence identity to the corresponding sequences in a TP53 gene from the non-human mammal.

The degree of sequence identity or sequence similarity between two sequences can be determined, for example, by comparing the two sequences using computer programs commonly employed for this purpose, such as global or local alignment algorithms. Non-limiting examples include BLASTp, BLASTn, Clustal W, MAFFT, Clustal Omega, AlignMe, Praline, GAP, BESTFIT, or another suitable method or algorithm. A Needleman and Wunsch global alignment algorithm can be used to align two sequences over their entire length, maximizing the number of matches and minimizes the number of gaps. Default settings can be used.

In some embodiments, expression of a p53 protein of the disclosure in a non-human animal or cell is driven by the endogenous p53 promoter from the non-human animal or cell. In some embodiments, expression of a p53 protein of the disclosure in a non-human animal or cell is driven by a promoter that is exogenous to the non-human animal or cell. In some embodiments, expression of a p53 protein of the disclosure in a non-human animal or cell is driven by a promoter that not the endogenous p53 promoter for the non-human animal or cell. In some embodiments, expression of a p53 protein of the disclosure in a non-human animal or cell is driven by a constitutive promoter. In some embodiments, expression of a p53 protein of the disclosure in a non-human animal or cell is driven by an inducible promoter. In some embodiments, expression of a p53 protein of the disclosure in a non-human animal or cell is driven by a cell type-specific promoter. In some embodiments, expression of a p53 protein of the disclosure in a non-human animal or cell is driven by a tissue-specific promoter.

### Candidate Therapeutic Agents

Engineered non-human animals and cells of the disclosure (e.g., Y220 HUPKI mice and cells therefrom) can be used to evaluate candidate therapeutic agents in a variety of in vitro and in vivo assays and cancer models as disclosed herein. A candidate therapeutic agent can be any agent that has been shown to possess anti-cancer activity, that is considered possibly to possess anti-cancer activity, or that is being tested for therapeutic (e.g., anti-cancer) activity. A candidate therapeutic agent can be, for example, a small molecule, a biologic, an antibody, a cell therapy, a radiotherapy, a metabolic therapy, etc.

A candidate therapeutic agent can be an experimental therapeutic agent, for example, a therapeutic agent that is not approved for use in humans, or that is being tested in a manner that is not approved for use in humans (e.g., a different dosing schedule, different cancer type or classification, or as part of a different combination therapy). A candidate therapeutic agent can be a therapeutic agent that is approved for use in treating cancer in humans.

Non-limiting examples of candidate therapeutic agents that can be evaluated using compositions and methods of the disclosure include p53 reactivating agents, chemotherapeutic agents, immunomodulators (e.g., immunotherapies, immune checkpoint inhibitors), AKT inhibitors, alkylating agents, anti-angiogenic agents, antibiotics, antifolates, anti-hormone therapies, anti-inflammatory agents, antimetabolites, anti-VEGF agents, apoptosis promoting agents, aromatase inhibitors, ATM regulators, biologic agents, BRAF inhibitors, BTK inhibitors, CAR-T cells, CDK inhibitors, cell growth arrest inducing-agents, cell therapies, chemotherapy, cytokine therapies, cytotoxic drugs, demethylating agents, differentiation-inducing agents, estrogen receptor antagonists, gene therapy agents, growth factor inhibitors, growth factor receptor inhibitors, HDAC inhibitors, heat shock protein inhibitors, hematopoietic stem cell transplantation (HSCT), hormones, hydrazine, kinase inhibitor, KRAS inhibitors, matrix metalloproteinase inhibitors, MEK inhibitors, mitotic inhibitors, mTOR inhibitors, multi-specific (e.g., bispecific) immune cell engagers, multi-specific (e.g., bispecific) killer cell engagers, multi-specific (e.g., bispecific) T cell engagers, nitrogen mustards, oncolytic viruses, oxazaphosphorines, plant alkaloids, platinum-based agents, proteasome inhibitors, purine analogs, purine antagonists, pyrimidine antagonists, radiation therapies, ribonucleotide reductase inhibitors, signal transduction inhibitors, surgery, taxanes, therapeutic antibodies, topoisomerase inhibitors, transgenic T cells, tyrosine kinase inhibitors, vinca alkaloids, and combination therapies comprising any combination thereof. In some embodiments, non-human animals or cells of the disclosure (e.g., Hupki-Y220C, Y220S, or Y220H mice or cells derived therefrom) can be used to evaluate p53 reactivating agents. Due to the prevalence of p53 mutations in many types of cancer, the reactivation or partial reactivation of wild type p53 function in a cancerous cell can be an effective therapy.

In some embodiments, non-human animals or cells of the disclosure (e.g., Hupki-Y220C mice or cells derived therefrom) allow testing of human Y220C-p53 reactivators in relevant in vivo and in vitro models, and identification of effective therapeutic agents and combination therapies. In some embodiments, non-human animals or cells of the disclosure (e.g., Hupki-Y220S mice or cells derived therefrom) allow testing of human Y220S-p53 reactivators in relevant in vivo and in vitro models, and identification of effective therapeutic agents and combination therapies. In some embodiments, non-human animals or cells of the disclosure (e.g., Hupki-Y220H mice or cells derived therefrom) allow testing of human Y220H-p53 reactivators in relevant in vivo and in vitro models, and identification of effective therapeutic agents and combination therapies. For example, compositions and methods disclosed herein can be used to identify and/or characterize p53 reactivators that inhibit tumor growth, prolong survival (e.g., progression-free survival), or a combination thereof.

Wild-type function of p53 mutants can be recovered by binding of the p53 mutant to a compound that can shift the folding-unfolding equilibrium towards the folded state, thereby reducing the rate of unfolding and destabilization.

In some embodiments, p53 reactivating agents can selectively bind to a p53 mutant and can recover wild-type activity of the p53 mutant including, for example, DNA binding function and activation of downstream targets involved in tumor suppression. In some embodiments, p53 reactivating agents selectively bind to the p53 Y220C mutant. The Y220C mutation can be temperature sensitive, e.g. can bind to DNA at lower temperature and be denatured at body temperature. In some embodiments, a p53 reactivating agent can stabilize the Y220C mutant to reduce the likelihood of denaturation of the protein at body temperature.

In some embodiments, p53 reactivating agents selectively bind to the p53 Y220S mutant. The Y220S mutation can be temperature sensitive, e.g. can bind to DNA at lower temperature and be denatured at body temperature. In some embodiments, a p53 reactivating agent can stabilize the Y220S mutant to reduce the likelihood of denaturation of the protein at body temperature. In some embodiments, p53 reactivating agents selectively bind to the p53 Y220H mutant. The Y220H mutation can be temperature sensitive, e.g. can bind to DNA at lower temperature and be denatured at body temperature. In some embodiments, a p53 reactivating agent can stabilize the Y220H mutant to reduce the likelihood of denaturation of the protein at body temperature.

Located in the periphery of the p53 (3-sandwich connecting β-strands S7 and S8, the aromatic ring of Y220 is an integral part of the hydrophobic core of the β-sandwich. Y220 mutations, such as the Y220C substitution, can be highly destabilizing, due to the formation of an internal surface cavity. In some embodiments, a p53 reactivating agent can bind to and occupy this surface crevice to stabilize the (3-sandwich, thereby restoring (e.g., partially restoring or fully restoring) wild-type p53 DNA-binding activity.

To determine the ability of a candidate p53 reactivating agent to bind and stabilize mutant p53, assays can be employed to detect, for example, a conformational change in the p53 mutant or activation of wild-type p53 targets. Conformational changes in p53 can be measured by, for example, differential scanning fluorimetry (DSF), isothermal titration calorimetry (ITC), nuclear magnetic resonance spectrometry (NMR), or X-ray crystallography. Additionally, antibodies specific for the wild type or mutant conformation of p53 can be used to detect a conformational change via, for example, immunoprecipitation (IP), immunofluorescence (IF), immunoblotting, or enzyme-linked immunosorbent assay (ELISA).

Methods used to detect the ability of the p53 mutant to bind DNA can include, for example, DNA affinity immunoblotting, modified enzyme-linked immunosorbent assay (ELISA), electrophoretic mobility shift assay (EMSA), fluorescence resonance energy transfer (FRET), homogeneous time-resolved fluorescence (HTRF), and a chromatin immunoprecipitation (ChIP) assay.

To determine whether a candidate p53 reactivating agent is able to reactivate the transcriptional activity of p53, the activation of downstream targets in the p53 signaling cascade can be measured (e.g., in vitro or in vivo). Activation of p53 effector proteins can be detected by, for example, immunohistochemistry (IHC-P), reverse transcription polymerase chain reaction (RT-PCR), RNA-seq, and western blotting. The activation of p53 can also be measured by the induction of apoptosis via the caspase cascade and using methods including, for example, Annexin V staining, TUNEL assays, pro-caspase and caspase levels, and cytochrome c levels. Another consequence of p53 activation is senescence, which can be measured using methods such as β-galactosidase staining. In some embodiments, non-human animals or cells of the disclosure (e.g., Hupki-Y220C, Y220S, or Y220H mice or cells derived therefrom) can be used to evaluate immunomodulatory effects of candidate therapeutic agents. For example, non-human animals or cells (e.g., Hupki-Y220C, Y220S, or Y220H mice, or cells derived therefrom) can be generated on an immunocompetent background (e.g., C57BL6, or other suitable strains), allowing evaluation of the effects of candidate therapeutic agents on the anti-cancer immune responses, such as immune cell infiltration into tumors and production of cytokines.

Compositions and methods disclosed herein can be used to identify and characterize p53 reactivators that increase anti-cancer immune responses. In addition to roles in cell-cycle arrest, apoptosis and metabolism, p53 can serve as a regulator of the immune system. For example, p53 can contribute to immune responses by directly activating regulators of immune signaling pathways, such as pathways that alter cytokine production, inflammation, immune cell chemotaxis, or a combination thereof In some embodiments, reactivation of p53 increases an anti-cancer immune response via an immune-regulatory role of p53. For example, in breast cancer patients, mutations in TP53 or loss of heterozygosity can correspond to low T-cell infiltration. In a p53-deficient mouse model of hepatocarcinoma, restoration of p53 expression can result in upregulation of inflammatory cytokines, an intratumoral innate immune response, and tumor regression. Deletion of p53 in a mouse model of pancreatic cancer can promote the recruitment of immune-suppressive myeloid cells T regulatory cells (Tregs), and attenuate anti-cancer CD4+ T helper 1 (Th1) and CD8+ T cell responses.

Compositions and methods disclosed herein can be used to identify and characterize the effects of immunomodulatory agents that promote anti-cancer immune responses, for example, immunotherapies, such as immune checkpoint inhibitors and immune cell therapies. An immune checkpoint inhibitor can bind to an immune checkpoint target and promote an anti-cancer immune response (e.g., by blocking an inhibitory signal and allowing the immune response to proceed). Non-limiting examples of immune checkpoint targets include 2B4, B7-1, B7-H3, BTLA, CD160, CTLA-4, DR6, Fas, LAG3, LAIR1, Ly108, PD-1, PD-L1, PD1H, TIGIT, TIM1, TIM2, and TIM3. An immune checkpoint inhibitor can be, for example, an antibody (or antigen-binding fragment or derivative thereof), a designed ankyrin repeat domain protein (DARPin), an aptamer, a small molecule, an affibody, an avimer, an adnectin, an anticalin, a Fynomer, a Kunitz domain, a knottin, a β-hairpin mimetic, a receptor, or a derivative thereof. Non-limiting examples of immune checkpoint inhibitors include Cemiplimab, Pembrolizumab, Nivolumab, Atezolizumab, Avelumab, Durvalumab, and Ipilimumab.

In some embodiments, compositions and methods disclosed herein can be used to identify and/or characterize the effects of immune stimulating agents, cytokine therapies, cytokine muteins, or cell therapies (e.g., allogeneic hematopoietic stem cell transplants, engineered immune cells such as CAR-T cells, NK cells, T cells, etc) on an anti-cancer immune response.

In some embodiments, compositions and methods of the disclosure are used to evaluate the effects of a monotherapy on an anti-cancer immune response. In some embodiments, compositions and methods of the disclosure are used to evaluate the effects of combination therapies on the anti-cancer immune response, for example, combinations of p53 reactivators and other agents, such as checkpoint inhibitors.

### Assays and Cancer Models

Non-human animals or cells of the disclosure (e.g., Hupki-Y220C, Y220S, or Y220H mice or cells derived therefrom) can be used to study various types of cancers, for example, to evaluate the effects of candidate therapeutic agents using in vitro and in vivo cancer models.

In some embodiments, the disclosure provides an assay comprising: (a) contacting a population of engineered non-human mammalian cells with a therapeutic agent, wherein the engineered non-human mammalian cells each comprise a nucleic acid that encodes a p53 protein, wherein the nucleic acid comprises a sequence with at least 90% sequence identity to exon 6 of human TP53, wherein the sequence with at least 90% sequence identity to exon 6 of human TP53 encodes an amino acid substitution at position 220 relative to human p53; and (b) after the contacting, observing an effect of the therapeutic agent on the population of engineered non-human mammalian cells.

In some embodiments, the observing the effect of the therapeutic agent comprises observing a viability of the population of engineered non-human mammalian cells in response to the therapeutic agent. In some embodiments, the observing the effect of the therapeutic agent comprises observing a metabolic activity of the population of engineered non-human mammalian cells in response to the therapeutic agent. In some embodiments, the observing the effect of the therapeutic agent comprises observing a conformation of the p53 protein in the population of engineered non-human mammalian cells. In some embodiments, the observing the effect of the therapeutic agent comprises determining an expression level of a gene in the population of engineered non-human mammalian cells. In some embodiments, the observing the effect of the therapeutic agent comprises determining an expression level of a p53 target gene in the population of engineered non-human mammalian cells. In some embodiments, the observing the effect of the therapeutic agent comprises determining an expression level of a protein in the population of engineered non-human mammalian cells.

In some embodiments, the therapeutic agent is a p53 modulating agent. In some embodiments, the therapeutic agent is a p53 activating agent. In some embodiments, the therapeutic agent is mutant p53 reactivating agent. In some embodiments, the therapeutic agent is a chemotherapeutic agent. In some embodiments, the therapeutic agent is a radiotherapy. In some embodiments, the therapeutic agent is an immunotherapy.

In some embodiments, cancer models disclosed herein involve generation of spontaneous tumors in non-human animals or cells of the disclosure (e.g., Hupki-Y220C, Y220S, or Y220H mice or cells derived therefrom). In some embodiments, tumors are induced by treating the non-human animals with mutagenic agents. In some embodiments, tumors are induced by treating the non-human animals with agents to generate additional specific mutations. In some embodiments, animals homozygous for a Y220 mutation are used (e.g., homozygous for a Y220C, Y220S, or Y220H substitution). In some embodiments, mice heterozygous for the Y220 (e.g., Y220C) mutation are used.

In some embodiments, cancer cell lines are generated from tumors that arise in the non-human animals (e.g., Hupki-Y220C, Y220S, or Y220H mice). The cell lines can be used in in vitro assays, for example, to test the anti-proliferative activity of candidate therapeutic agents. The cell lines can be used to develop in vivo models, for example, the cells can be injected into recipient animals, such that tumors grow in the recipient animals (e.g., mice). The recipient animals can be syngeneic to the cells. In some embodiments, the recipient animals are allogenic to the cells.

Cancer models utilizing non-human animals or cells of the disclosure (e.g., Hupki-Y220C, Y220S, or Y220H mice or cells derived therefrom) can be used to test various aspects of candidate therapeutic agents. In some embodiments, cancer models utilizing non-human animals or cells of the disclosure (e.g., Hupki-Y220C, Y220S, or Y220H mice or cells derived therefrom) are used to study pharmacodynamics, pharmacokinetics, or a combination thereof of a candidate anti-cancer therapeutic.

In some embodiments, cancer models utilizing non-human animals or cells of the disclosure (e.g., Hupki-Y220C, Y220S, or Y220H mice or cells derived therefrom) are used to study the effects of a candidate anti-cancer therapy on gene expression, signaling pathway activity, apoptosis, cell cycle progression, cancer cell growth and proliferation, ubiquitination, p53 conformation, or a combination thereof.

In some embodiments, cancer models utilizing non-human animals or cells of the disclosure (e.g., Hupki-Y220C, Y220S, or Y220H mice or cells derived therefrom) are used to study the effects of a candidate anti-cancer therapy on survival, progression-free survival, tumor growth inhibition, tumor regression, or a combination thereof.

In some embodiments, cancer models utilizing non-human animals or cells of the disclosure (e.g., Hupki-Y220C, Y220S, or Y220H mice or cells derived therefrom) are used to study the effects of a candidate therapeutic agent on an anti-cancer immune response, for example, inflammation, cytokine production (e.g., pro-inflammatory and/or anti-inflammatory cytokine production), immune cell infiltration, immune cell exhaustion, immune cell reactivation, immune cell proliferation, antigen-specific anti-cancer immune responses, or a combination thereof.

In some embodiments, cancer models utilizing non-human animals or cells of the disclosure (e.g., Hupki-Y220C, Y220S, or Y220H mice or cells derived therefrom) are used to study the effects of a candidate therapeutic agent on angiogenesis.

In some embodiments, compositions and methods of the disclosure are used to evaluate a monotherapy. In some embodiments, compositions and methods of the disclosure are used to evaluate a combination therapy, for example, a combination of a p53 reactivating agent and another agent, such as a checkpoint inhibitor.

In some embodiments, compositions and methods of the disclosure are used to identify a suitable route of administration for an anti-cancer therapeutic. Non-limiting examples of routes of administration that can be identified include local administration, systemic administration, administration in a rapid release formulation, administration in an extended-release formulation, administration in an intermediate-release formulation, oral administration, topical administration, parenteral administration, intravenous injection, intravenous infusion, subcutaneous injection, intramuscular injection, intradermal injection, intraperitoneal injection, intracerebral injection, subarachnoid injection, intraocular injection, intraspinal injection, intrasternal injection, ophthalmic administration, endothelial administration, intranasal administration, intrapulmonary administration, rectal administration, intraarterial administration, intrathecal administration, inhalation, intratumoral administration, intralesional administration, intradermal administration, epidural administration, absorption through epithelial or mucocutaneous linings (e.g., oral mucosa, rectal and intestinal mucosa), intracapsular administration, subcapsular administration, intracardiac administration, transtracheal administration, subcuticular administration, subarachnoid administration, subcapsular administration, intraspinal administration, intrasternal administration, and any combination thereof.

In some embodiments, compositions and methods of the disclosure are used to identify a suitable dosage and/or or dosing schedule for an anti-cancer therapeutic.

Non-human animals or cells of the disclosure (e.g., Hupki-Y220C, Y220S, or Y220H mice or cells derived therefrom) can be used to study any type of cancer. For example, tumors arising from a tissue of origin can be identified, and optionally a cell line can be generated (e.g., for use in a syngeneic mouse model of a particular cancer). In some embodiments, a model of a sarcoma is developed using a sarcoma that develops in the non-human animal (e.g., Hupki-Y220C, Y220S, or Y220H mice). In some embodiments, a model of a lymphoma is developed using a lymphoma that develops in the non-human animal (e.g., Hupki-Y220C, Y220S, or Y220H mice). In some embodiments, a model of a myeloma is developed using a myeloma that develops in the non-human animal (e.g., Hupki-Y220C, Y220S, or Y220H mice). In some embodiments, a model of a leukemia is developed using a leukemia that develops in the non-human animal (e.g., Hupki-Y220C, Y220S, or Y220H mice). In some embodiments, a model of an adenoma is developed using an adenoma that develops in the non-human animal (e.g., Hupki-Y220C, Y220S, or Y220H mice). In some embodiments, a model of a carcinoma is developed using a carcinoma that develops in the non-human animal (e.g., Hupki-Y220C, Y220S, or Y220H mice).

The cancer models can be developed using non-human animals of any suitable genetic background. In some embodiments, a cancer model is developed using Hupki-Y220C, Y220S, or Y220H mice in the C57BL6 genetic background. In some embodiments, a cancer model is developed using Hupki-Y220C, Y220S, or Y220H mice in the a non-C57BL6 background, for example, as disclosed herein. In some embodiments, a cancer model is developed using a Hupki-Y220C, Y220S, or Y220H non-human mammal that is not a mouse, for example, a rat, rodent, rabbit, guinea pig, hamster, or pig.

Also disclosed herein is a method of evaluating a therapeutic agent, comprising administering a therapeutically-effective amount of the therapeutic agent to a subject with a cancer, wherein the cancer comprises an engineered non-human mammalian cell, wherein the engineered non-human mammalian cell comprises a nucleic acid that encodes a p53 protein, wherein the nucleic acid comprises a sequence with at least 90% sequence identity to exon 6 of human TP53, wherein the sequence with at least 90% sequence identity to exon 6 of human TP53 encodes an amino acid substitution at position 220 relative to human p53.

In some embodiments, the cancer is a sarcoma. In some embodiments, the cancer is a carcinoma. In some embodiments, the cancer is a leukemia. In some embodiments, the cancer is a lymphoma. In some embodiments, the cancer is a myeloma.

In some embodiments, the subject is a mouse. In some embodiments, the subject is syngeneic to the engineered non-human mammalian cell.

In some embodiments, the method further comprises administering a therapeutically-effective amount of an additional therapeutic agent to the subject. In some embodiments, the additional therapeutic agent is an immunomodulator. In some embodiments, the additional therapeutic agent is an immune checkpoint inhibitor. In some embodiments, the additional therapeutic agent is a chemotherapeutic. In some embodiments, the additional therapeutic agent is a radiotherapy.

In some embodiments, the method further comprises determining an effect of the therapeutic agent on survival of the subject. In some embodiments, the method further comprises determining an effect of the therapeutic agent on tumor volume in the subject. In some embodiments, the method further comprises determining an effect of the therapeutic agent on an anti-cancer immune response in the subject. In some embodiments, the method further comprises determining a conformation of the p53 protein in the subject in response to the therapeutic agent. In some embodiments, the method further comprises determining an expression level of a gene in the subject in response to the therapeutic agent. In some embodiments, the method further comprises determining an expression level of a p53 target gene in the subject in response to the therapeutic agent. In some embodiments, the method further comprises determining an expression level of a protein in the subject in response to the therapeutic agent. In some embodiments, the method further comprises evaluating a pharmacokinetic parameter of the therapeutic agent in the subject in response to the therapeutic agent.

Compositions and methods of the disclosure can be used to study and develop models of, for example, acute lymphoblastic leukemia, acute myeloid leukemia, adrenocortical carcinoma, AIDS-related cancers, AIDS-related lymphoma, anal cancer, appendix cancer, astrocytomas, basal cell carcinoma, bile duct cancer, bladder cancer, bone cancers, brain tumors, such as cerebellar astrocytoma, cerebral astrocytoma/malignant glioma, ependymoma, medulloblastoma, supratentorial primitive neuroectodermal tumors, visual pathway and hypothalamic glioma, breast cancer, bronchial adenomas, Burkitt lymphoma, carcinoma of unknown primary origin, central nervous system lymphoma, cerebellar astrocytoma, cervical cancer, childhood cancers, chronic lymphocytic leukemia, chronic myelogenous leukemia, chronic myeloproliferative disorders, colon cancer, cutaneous T-cell lymphoma, desmoplastic small round cell tumor, endometrial cancer, ependymoma, esophageal cancer, Ewing's sarcoma, germ cell tumors, gallbladder cancer, gastric cancer, gastrointestinal carcinoid tumor, gastrointestinal stromal tumor, gliomas, hairy cell leukemia, head and neck cancer, heart cancer, hepatocellular (liver) cancer, Hodgkin lymphoma, Hypopharyngeal cancer, intraocular melanoma, islet cell carcinoma, Kaposi sarcoma, kidney cancer, laryngeal cancer, lip and oral cavity cancer, liposarcoma, liver cancer, lung cancers, such as non-small cell and small cell lung cancer, lymphomas, leukemias, macroglobulinemia, malignant fibrous histiocytoma of bone/osteosarcoma, medulloblastoma, melanomas, mesothelioma, metastatic squamous neck cancer with occult primary, mouth cancer, multiple endocrine neoplasia syndrome, myelodysplastic syndromes, myeloid leukemia, nasal cavity and paranasal sinus cancer, nasopharyngeal carcinoma, neuroblastoma, non-Hodgkin lymphoma, non-small cell lung cancer, oral cancer, oropharyngeal cancer, osteosarcoma/malignant fibrous histiocytoma of bone, ovarian cancer, ovarian epithelial cancer, ovarian germ cell tumor, pancreatic cancer, pancreatic cancer islet cell, paranasal sinus and nasal cavity cancer, parathyroid cancer, penile cancer, pharyngeal cancer, pheochromocytoma, pineal astrocytoma, pineal germinoma, pituitary adenoma, pleuropulmonary blastoma, plasma cell neoplasia, primary central nervous system lymphoma, prostate cancer, rectal cancer, renal cell carcinoma, renal pelvis and ureter transitional cell cancer, retinoblastoma, rhabdomyosarcoma, salivary gland cancer, sarcomas, skin cancers, skin carcinoma merkel cell, small intestine cancer, soft tissue sarcoma, squamous cell carcinoma, stomach cancer, T-cell lymphoma, throat cancer, thymoma, thymic carcinoma, thyroid cancer, trophoblastic tumor (gestational), cancers of unknown primary site, urethral cancer, uterine sarcoma, vaginal cancer, vulvar cancer, Waldenström macroglobulinemia, Wilms tumor, and any combination thereof.

In some embodiments, compositions and methods of the disclosure are used to study a sarcoma. In some embodiments, compositions and methods of the disclosure are used to study a lymphoma. In some embodiments, compositions and methods of the disclosure are used to study an adenoma. In some embodiments, compositions and methods of the disclosure are used to study a carcinoma. In some embodiments, compositions and methods of the disclosure are used to study a myeloma. In some embodiments, compositions and methods of the disclosure are used to study a leukemia.

### EXAMPLES

### Example 1

### Generation of a Human p53-Y220C Knock-In Syngeneic Mouse Model

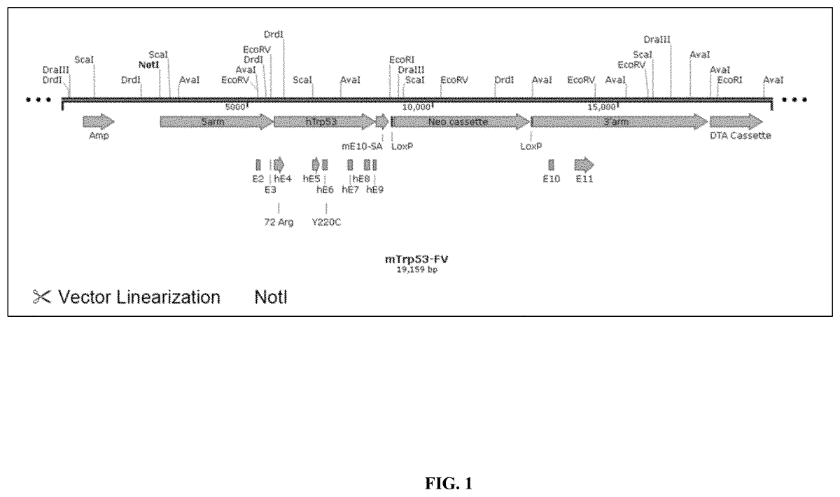

Human p53 knock-in (“Hupki”) mice were generated in which the human binding domain (exons 4-9) was knocked-in to the mouse p53 gene, with the amino acid at position 220 mutated from a tyrosine to a cysteine (Y220C). Exons 4-9 of the mTrp53 gene (GenBank accession number: NM_011640.3, ENSMUSG00000059552) were replaced by the corresponding human genomic DNA fragment containing hTP53 exons 4˜9, along with the Y220C mutation (CDS mutation c.659A>G). Polymorphic codon 72 that encodes arginine instead of proline (c. 215C>G, p. P72R) was also included in the human sequence. Homology arms were generated by PCR using BAC clone RP23-51O13 and RP23-243M15 from the C57BL/6J library as a template. A diagram of the targeting construct is provided in FIG. 1. C57BL/6 embryonic stem (ES) cells were used for electroporation of the targeting construct. The targeting construct was introduced into competent ES cells, followed by appropriate drug selection, introduction into host embryos, and transfer into surrogate mothers. The subsequently generated chimeras (FO) mice were 100% ES derived and germline transmitted. Male chimeras (F0) were identified and subsequently mated to C57BL/6 females to obtain F1mice.

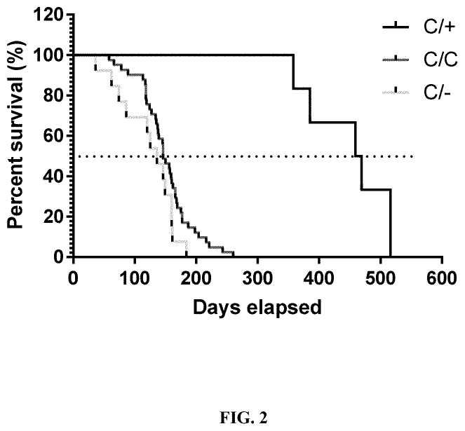

Mice homozygous for the Y220C mutation succumbed to lymphomas and sarcomas within 6 months, mice heterozygous for mutant Y220C succumbed to tumor burden by 1.5 years (FIG. 2). All animal work was conducted in a specific pathogen free (SPF), AAALAC accredited and OLAW assured facility.

### Example 2

### Generation of Cell Lines from Hupki-Y220C Mice

Cell lines were generated from tumors (sarcomas and lymphomas) that arose in mice homozygous for the Y220C mutation generated in Example 1. Sarcoma tumors were taken from Y220C/C homozygous mice, finely chopped with surgical blades, added to 3mL trypsin solution, and incubated at room temperature for 10 minutes. 10 mL of growth medium (DMEM with 10% FBS and 1% penicillin + streptomycin solution) was then added, and cells were incubated at 37 ° C. for two days. Media was changed the next day and cells were cultured in 10 mL fresh medium until sufficient density was achieved for cryopreservation. Cells were frozen in cryopreservation medium (DMEM with 20% FBS and 10% DMSO).

For thymic lymphomas, the thymus cells were dissociated using bend needles and suspended in 10 mL growth media (RPMI with 10% FBS and 1% penicillin + streptomycin solution). Cells were washed with media, transferred to a flask, and grown for 48 hrs, at which time more fresh media was added. After 72 hrs, cell suspensions were split. Cells were cultured until sufficient density was achieved for cryopreservation. Cells were frozen in cryopreservation medium (RPMI with 20% FBS and 10% DMSO).

### Example 3

### Cell Lines Generated from Hupki-Y220C Mice Can Be Used for Screening Activity of p53 Reactivating Compounds

Compound 1 is a small molecule that binds to and reactivates mutant Y220C p53 by changing the mutant conformation of mutant p53 to the corresponding wild type conformation of p53. The single tyrosine to cysteine amino acid change can create a small crevice in the mutant p53 protein, making it thermally unstable and unable to effectively interact with DNA. Compound 1 can restore wild type p53 structure and reactivate its function by selectively binding into this crevice.

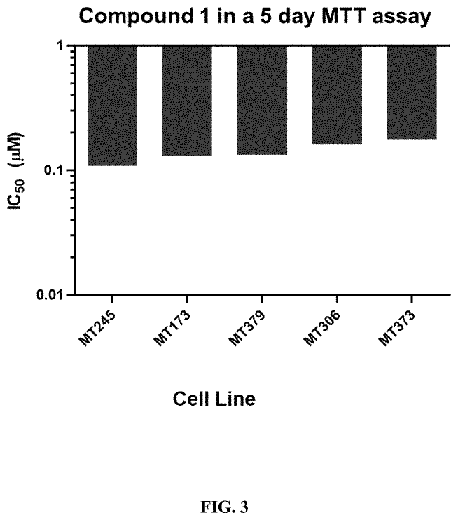

Five representative lymphoma and sarcoma cell lines generated from Hupki-Y220C mice in Example 2 were tested for sensitivity to Compound 1 in a 5-day MTT assay, which is a colorimetric assay for assessing cell metabolic activity based on reduction of MTT (3-(4,5-dimethylthiazol-2-yl)-2,5-diphenyltetrazolium bromide). The cell lines were designated MT245, MT1713, MT379, MT306, and MT373. The cell lines were sensitive to Compound 1 as shown in FIG. 3. Treatment with Compound 1 resulted in an anti-proliferative effect, with IC₅₀ values as follows: MT245 (0.109 μM), MT173 (0.130 μM), MT379 (0.133 μM), MT306 (0.162 μM), and MT373 (0.175 μM). These data demonstrate that Compound 1 has an anti-proliferative effect on tumors with Y220C mutations in p53. These data also demonstrate that tumors and cell lines from Hupki-Y220C mice can be used for testing p53 reactivating compounds for therapeutic effects (e.g., anti-proliferative effects).

### Example 4

### In Vivo Syngeneic Mouse Tumor Models Utilizing Cell Lines Generated in Hupki-Y220C Mice

C57BL/6 mice were acclimatized for 4 weeks and were 8-10 weeks old at initiation of study. Animals were group housed (N=5) in ventilated cages. Fluorescent lighting was provided on a 12-hour cycle (6:30 am-6:30 pm). Temperature and humidity were monitored, recorded daily, and maintained between 68-72° F. (20-22.2° C.) and 30-70% humidity, respectively. 2920X.10 18% soy irradiated rodent feed and autoclaved acidified water (pH2.5-3) were provided ad libitum.

Tumor cell lines established from Hupki-Y220C homozygous mice as described in example 2 were cultured in DMEM media with 10% fetal bovine serum. Cells were spun by centrifuge and resuspended in 50% PBS:50 Matrigel Matrix at a concentration of 5,000,000 viable cells/200 μL for MT373 cells, and 1,000,000 viable cells/100 μL for MT245 cells. Cells were prepared for injections by drawing the cell suspension into a 1 mL tuberculin syringe fitted with a 25G ⅝″ needle. Individual mice were manually restrained, the site of injection (right flank) was disinfected with a 70% ethanol swab, and 100 μL or 200 μL of cell suspension was injected subcutaneously. Implanted animals were monitored for palpable tumors. Six days post-implant, animals with palpable tumors had their tumor sizes determined via digital caliper. Mice were selected and randomized into treatment groups according to tumor size.

Tumor volume was calculated using the following equation: (longest diameter x shortest diameter²)/2. Individual tumor volumes were recorded at study initiation. The calculation for percent tumor growth inhibition (TGI) is as follows: [1−(Tₜ−T₀/Cₜ−C₀))]×100, where Cₜ is the mean tumor volume of the vehicle control group at time t, C₀ is the mean tumor volume of the vehicle control group at time 0, and T is the mean tumor volume of the treatment group. Tumor regression was determined with the equation [(T₀−Tₜ)/T₀]×100 using the same definitions.

All studies were conducted following an approved IACUC protocol, and all experimental data management and reporting procedures were in strict accordance with guidelines and standard operating procedures.

### Example 5

### Effects of a p53 Reactivating Compound in Syngeneic Mouse Tumor Models Utilizing Cell Lines Generated in Hupki-Y220C Mice

Compound 1 was tested in two syngeneic mouse models of soft tissue sarcoma, utilizing the MT373 and MT245 cell lines generated in Example 2. The cell lines were injected subcutaneously into C57BL/6 mice as described in example 4. These tumor cell lines were found to grow well when implanted subcutaneously into C57BL/6 female mice, demonstrating that cell lines from Hupki-Y220C mice can be used to establish syngeneic tumor models.

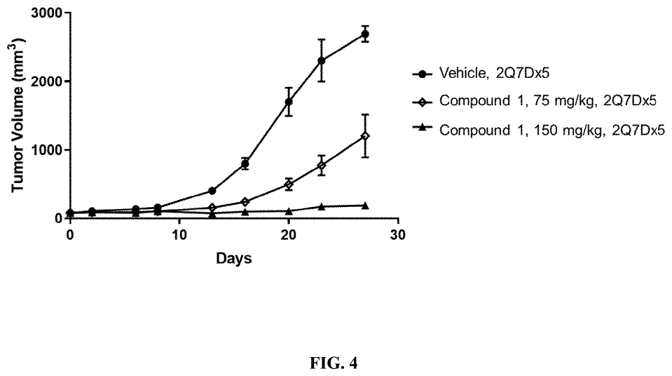

Female C57BL/6 mice bearing MT373 syngeneic tumors were administered Compound 1 at doses of 75 and 150 mg/kg twice daily once a week (2Q7D). FIG. 4 illustrates changes in tumor volume over time. On day 27 of the study, mice dosed with 75mg/kg exhibited 57% tumor growth inhibition (TGI), and mice dosed with 150 mg/kg exhibited 96% TGI. One of ten mice dosed with Compound 1 at 150 mg/kg 2Q7Dx5 had a tumor regress to a complete cure by day 20. Compound 1 was well tolerated with body weight gains across the course of the study. All dosing was discontinued on day 102 and the remaining mice with no palatable tumors were monitored for survival until day 185.

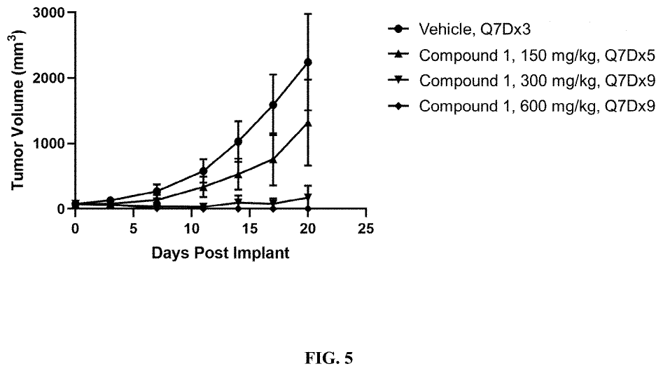

Mice bearing MT245 sarcoma tumor xenografts were administered Compound 1 at 150, 300, and 600 mg/kg once a week (Q7D) which resulted in 43% TGI, 96% TGI, and 100% regression, respectively, as shown in FIG. 5. Tumors in all 10 mice administered the dose of 600 mg/kg Q7Dx9 regressed completely within 17 days of initiating treatment. These mice were dosed for 55 days, after which dosing was ceased and mice were monitored for tumor regrowth for 157 days. The tumors did not regrow within the 157 days of the study duration, indicating durable cures in this mouse syngeneic model of sarcoma.

These data demonstrate that Compound 1 is effective as a treatment for tumors bearing Y220C mutations in p53. These data also demonstrate that syngeneic mouse cancer models can be established utilizing cell lines generated from Hupki-Y220C mice, and that these models can be used to evaluate candidate anti-cancer therapeutics, such as p53 reactivating compounds.

### Example 6

### Pharmacodynamics and Pharmacokinetics of a p53 Reactivating Compound in a Syngeneic Mouse Tumor Model

The Pharmacodynamic (PD) and Pharmacokinetic (PK) relationship of Compound 1 was tested in a mouse syngeneic model of sarcoma utilizing the MT373 cell line generated in example 2. Cells were injected subcutaneously into C57BL/6 mice as described in example 4. Compound 1 was administered orally at 75 or 150 mg/kg twice in one day (BID×1), and was well tolerated through the dosing period. Tumors and plasma were harvested for PD/PK analysis 8, 24, 48, 72, 96, and 144 hours after the first dose. Plasma concentrations of Compound 1 are presented in TABLE 2.

Levels of wild type conformation p53, mutant conformation p53, and total p53 within tumors were determined by ELISA. 96-well ELISA plates were coated via overnight incubation at 4° C. with antibodies specific for wild type (WT) p53 (150 ng/well; clone PAb1620), mutant p53 (250 ng/well; clone PAb240), or total p53 (62.5 ng/well; clone 1C12). Plates were washed with wash buffer (PBS + 0.05% Tween-20), treated with blocking buffer (PBS+1% BSA+0.05% Tween-20) for 1 h, and then washed. Samples were diluted in blocking buffer such that the required protein amount was added to the plate in a 100 μL volume (WT p53 7.5 μg; mutant p53 2.5 μg; total p53 2.5 μg). Samples were incubated overnight at 4° C. with shaking. Plates were again washed and treated with detection antibody (clone D2H90) diluted in blocking buffer (0.625 μg/mL for mutant p53; 0.156 μg/mL for total p53, and 0.3 μg/mL for WT p53) for 1 h. Plates were washed and then incubated in anti-rabbit-HRP (1:100) diluted in blocking buffer for 1 h. Plates were washed, the reaction developed using TMB for approximately 5 minutes, and the reaction quenched with 0.16 M sulfuric acid. Plates were read on a plate reader at 450 nm. A background measurement was subtracted from the treated samples signal and they were normalized to their respective vehicle controls.

The results of the ELISA are provided in TABLE 2. Dose-responsive decreases in mutant conformation p53 and increases in wild type conformation p53 were observed upon treatment with Compound 1. For example, mice administered Compound 1 at 75 mg/kg exhibited a 53.91% decrease in mutant conformation p53 and a 1.25 fold increase in wild type conformation p53 at 8h, and mice administered Compound 1 at 150 mg/kg exhibited an 82.3% decrease in mutant conformation p53 and a 1.45 fold increase in wild type conformation p53 at 8 h, relative to vehicle-treated animals. The changes in p53 conformation were associated with high plasma concentrations of Compound 1 at Cmax (8375 and 11435 ng/mL), and as concentrations of Compound 1 decreased, p53 returned to the mutant conformation. The higher dose of Compound 1 was associated with a greater magnitude and duration of p53 conformation alteration. These data demonstrate that Compound 1 can alter Y220C mutant p53 from a mutant to a wild type conformation. These data also demonstrate that syngeneic mouse cancer models utilizing cell lines generated from Hupki-Y220C mice can be used to study pharmacodynamics and pharmacokinetics of candidate anti-cancer therapeutics, such as p53 reactivating compounds.

TABLE 2
Plasma and tumor concentrations of Compound 1, changes in intra-tumor
mutant and wild type conformation p53, and expression of p53 target genes in mice harboring
MT373 tumors following treatment with Compound 1 orally at 75 or 150 mg/kg twice daily for 1
day (BIDx1).
TABLE 2

| | | | | Fold Change Over Vehicle Control or | | | | | |
|---|---|---|---|---|---|---|---|---|---|
| | | Comp 1 | Comp 1 | Percent Reduction Relative to Vehicle Control (%) | | | | | |
| | Time | Plasma | Tumor | Mutant | | | | | |
| | point | Conc. | Conc. | p53 | WT p53 | Total p53 | p21 | Mdm2 | Mic-1 |
| Group | (h) | (ng/mL) | (ng/g) | Protein | Protein | Protein | mRNA | mRNA | mRNA |
| 75 | 8 | 8375 | 30853 | 53.91% | 1.25 | 35.06% | 5.43 | 6.16 | 1.74 |
| mg/kg | 24 | 1843 | 12035 | 79.5% | 1.13 | 72.38% | 4.04 | 2.44 | 1.14 |
| BIDx1 | 48 | 40 | 318 | 67.23% | 1.02 | 63.03% | 1.43 | 1.12 | 0.85 |
| | 72 | 0 | 97 | 16.87% | 1.11 | 19.65% | 1.18 | 1.08 | 0.65 |
| | 98 | 0 | 0 | 0.00% | 1.13 | 0.00% | 0.99 | 1.11 | 1.05 |
| | 144 | 0 | 0 | 45.58% | 0.88 | 44.67% | 0.98 | 0.85 | 0.88 |
| 150 | 8 | 11435 | 57645 | 82.3% | 1.45 | 56.64% | 6.73 | 8.70 | 3.07 |
| mg/kg | 24 | 5986 | 67778 | 93.74% | 1.08 | 81.75% | 9.61 | 6.88 | 2.29 |
| BIDx1 | 48 | 268 | 2436 | 81.64% | 0.94 | 81.46% | 1.87 | 1.43 | 1.14 |
| | 72 | 7 | 59 | 58.64% | 0.86 | 60.43% | 1.33 | 1.33 | 0.93 |
| | 98 | 3 | 23 | 0.00% | 0.95 | 0.60% | 1.01 | 1.12 | 0.53 |
| | 144 | 0 | 0 | 34.14% | 0.86 | 32.4% | 0.85 | 1.16 | 1.00 |

Binding of Compound 1 to mutant p53 can induce a change to a wild-type conformation, thereby permitting binding of p53 to DNA and initiating expression of p53 target genes. The effect of Compound 1 on expression of p53 target genes in tumor tissue was measured by real time PCR. Tumor samples with the required weight were lysed in RNA isolation buffer with 10 μL/mL of β-mercaptoethanol, using a bead mill. Total RNA was further purified from the lysate via column purification with DNase digestion. RNA concentration was measured by Spectrophotometer. Individual gene expression analysis was done by one-step TaqMan-based real-time RT-qPCR. Purified total RNA was diluted to 2.5 ng/μL in DNase- and RNase-free water, and 10 ng was used for each RT-qPCR assay in a 20 μL reaction. In each assay, a probe detection kit was used along with a specific primer/probe set for each corresponding gene.

Expression of the reference gene (mouse Gapdh) in the ratio to the vehicle control was calculated by the ΔCt method with a normalization to the total RNA input. Expression of a gene of interest relative to the reference gene was calculated by the ΔCt method, and the expression of a gene of interest relative to the reference gene in the ratio to the vehicle control was calculated by the ΔΔCt method. Analysis of downstream p53 transcriptional targets p21, Mdm2 and Mic-1 showed dose responsive increases in p21 and Mdm2, as shown in TABLE 2. 8 hours after the initial dose, mice treated with 75 and 150 mg/kg of Compound 1 exhibited 5.4 and 6.7-fold increases in p21 mRNA, and 6.2 and 8.7-fold increases in Mdm2 mRNA, respectively. Mic1 mRNA levels only increased more than 2-fold in the 150 mg/kg group, with a 3.07-fold increase over vehicle at 8 h. These results are consistent with Compound 1 promoting binding of Y220C-mutant p53 to DNA, and initiating expression of p53 target genes. These data also demonstrate that syngeneic mouse cancer utilizing cell lines generated from Hupki-Y220C mice can be used to study transcriptional effects of candidate anti-cancer therapeutics, such as p53 reactivating compounds.

Changes in the expression of additional genes were evaluated by profiling panels of genes upstream and downstream of p53 and NFkB. Pathway profiling was done using SYBR Green-based real-time qPCR after reverse transcription. cDNA was synthesized from 500 ng purified total RNA of each tumor sample using a first strand cDNA synthesis kit. cDNA and SYBR green master mix were added to mouse p53 signaling pathway array plates and/or mouse NFkB signaling pathway array plates. At least 3 samples in each group were used for the profiling. Reactions were conducted and data collected using a benchtop real time PCR instrument. The average Ct values of 5 reference genes was used to normalize plate-to-plate variation. Similar results were achieved by the ΔΔCt method using 5 housekeeping genes as the first reference control and the vehicle group as the second reference control. A cutoff of fold change=2 and p-value=0.05 was applied to curate the data, with a consideration of eliminating some low expression genes (Ct<30).

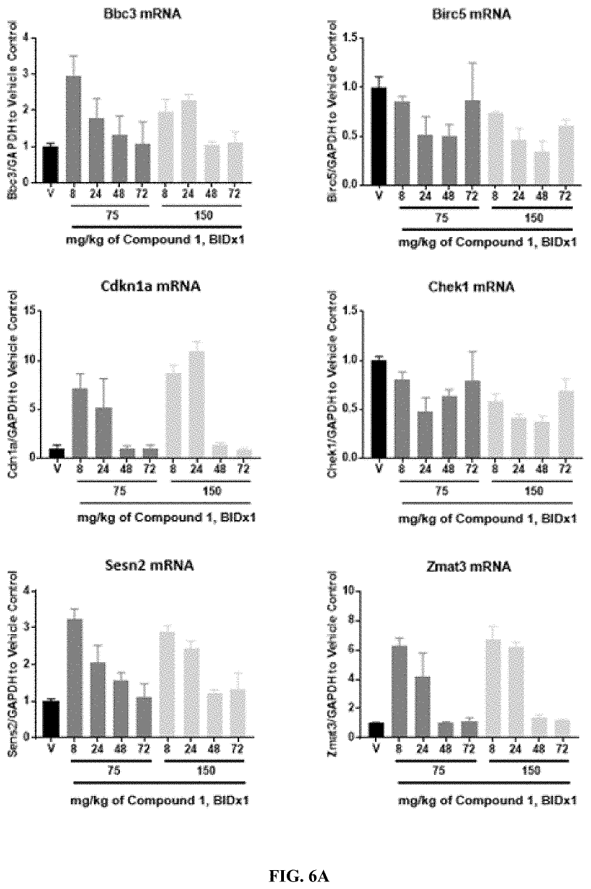

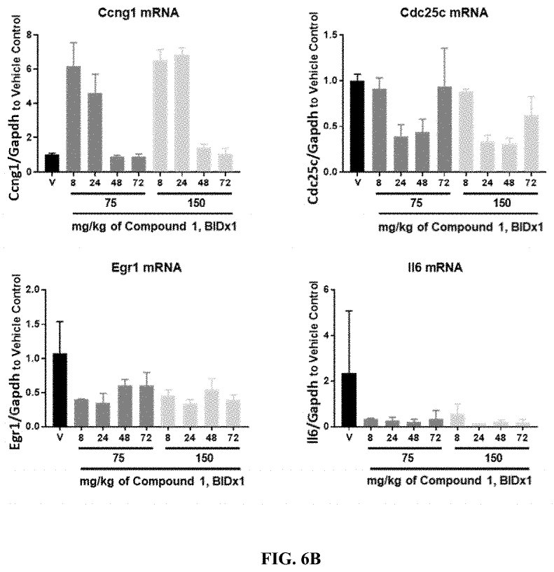

Expression levels were determined for 84 p53-related genes, some of which are regulated by p53 activity, upstream or downstream of p53. Measurement of target gene transcripts downstream of p53 showed that administering Compound 1 led to changes in genes related to apoptosis at several timepoints. For example, mice administered Compound 1 exhibited a 1.8-fold increase (75 mg/kg) or 2.27-fold increase (150 mg/kg) in Bbc3, and a 45.92% decrease (75 mg/kg) or 53.57% decrease (150 mg/kg) in Birc5, at 24 hours post-treatment. Changes were also observed in genes related to cell cycle control at several time points. For example, mice administered 75 mg/kg of Compound 1 exhibited fold increases or percentage decreases in the expression of Ccng1 (4.63-fold increase), Cdc25c (60.54% decrease), Cdk1 (59.92% decrease), Cdkn1a (5.16-fold increase), Chek1 (52.36% decrease), and Zmat3 (4.14-fold increase) at 24 hours. Mice administered 150 mg/kg of Compound 1 exhibited also exhibited changes in expression of these genes as follows: Ccng1 (6.84-fold increase), Cdc25c (41.75% decrease), Cdk1 (64.58% decrease), Cckn1a (10.93-fold increase), Chek1 (58.77% decrease), Zmat3 (6.17-fold increase) at 24 hours. Other genes that were significantly upregulated or downregulated in tumors of mice treated with Compound 1 at 75 and 150 mg/kg BIDx1 were related to growth and proliferation (e.g., Egfr—28.18% decrease and 47.37% decrease at 24 h, respectively), inflammation and immune response (e.g., IL6—70.78% decrease and 85.31% decrease at 24 h, respectively), ubiquitination (e.g., Mdm2—2.76-fold increase and 5.96-fold increase at 24 h, respectively) and cell growth (e.g., Sesn2—2.05-fold increase and 2.45-fold increase at 24 h, respectively). FIG. 6A and FIG. 6B show relative expression of 10 representative 10 genes from this panel. FIG. 6A illustrates expression of Bbc3, Birc5, Cdkn1a, Chek1, Sesn2, and Zmat3. FIG. 6B illustrates expression of Ccng1, Cdc25c, Egr1, and IL6.

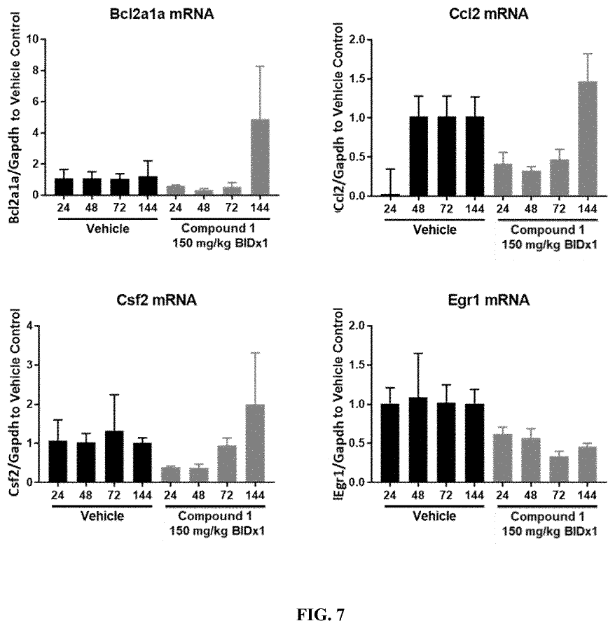

Expression levels of genes upstream and downstream of NF-κB were also evaluated to determine the effect of p53 reactivation on NF-κB signaling, which can affect inflammatory responses, cellular growth, and apoptosis. Mice were treated with 150 mg/kg of Compound 1, BIDx1. Several genes were downregulated at various timepoints that were related to an immune response, for example, Cc12 (57.69% at 24 h, 67.28% at 48 h, 52.53% at 72 h), Csf2 (61.29% at 24 h, 62.24% at 48 h), Ifnγ (35.82% at 24 h, 43.46% at 48 h, 22.04% at 72 h), Il1α (13.50% at 24 h, 28.88% at 48 h, 63.60% at 72 h), and Il1β (63.25% at 24 h, 18.73% at 48 h, 84.09% at 72 h). Egfr, a gene involved in proliferation, and Fasl, a gene involved in apoptosis, were also downregulated at several timepoints (28.45% at 24 h, 24.42% at 48 h, 41.90% at 144 h) and (31.85% at 24 h, 42.85% at 72 h), respectively. FIG. 7 shows changes in expression of four genes relative to the vehicle control at 24, 48, 72, and 144 h.

These results indicate that administration of Compound 1 alters the conformation of Y220C p53 from a mutant conformation to a wild type conformation, and results in alteration of signaling in the p53 pathway and NF-κB pathway. These data also demonstrate that syngeneic mouse cancer models utilizing cell lines generated from Hupki-Y220C mice can be used to study transcriptional effects of candidate anti-cancer therapeutics, such as p53 reactivating compounds.

### Example 7

### Synergistic Effects of Co-Administering a p53 Reactivating Compound and an Immune Checkpoint Inhibitor in Syngeneic Mouse Tumor Models

Combination therapies that included a p53 reactivating compound and an immune checkpoint inhibitor were tested in a mouse syngeneic tumor models utilizing the MT373 and MT245 cell lines generated in example 2. Cells were injected subcutaneously into C57BL/6 mice as described in example 4.

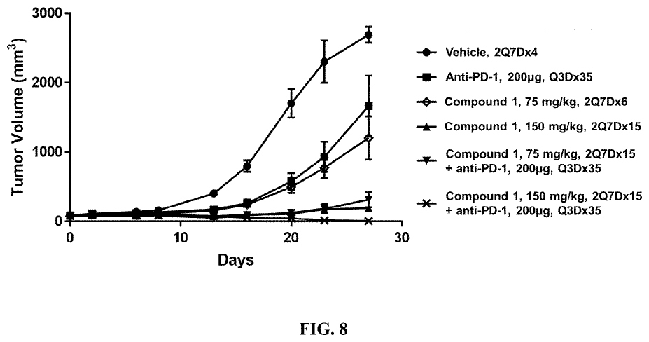

Compound 1 was administered to mice bearing MT373 tumors at 75 or 150 mg/kg, twice daily, once per week (2Q7D). Mice receiving anti-PD-1 were given intraperitoneal injections of 200 μg of anti-PD-1 (clone BE0146), once every three days (Q3D). As shown in FIG. 8, combination therapy increased efficacy, including from 96% tumor growth inhibition (TGI) to 92% regression for mice given 150 mg/kg of Compound 1 at day 27. All dosing was discontinued on day 102 and the remaining mice with no palatable tumors were monitored for survival until day 185. The median survival time (defined as median time taken for tumors in a group to reach 2000 mm³) was significantly increased in mice receiving the high dose of Compound 1 (150 mg/kg) in combination with anti-PD-1, to >185 days, compared to 20.9 days for vehicle control mice.

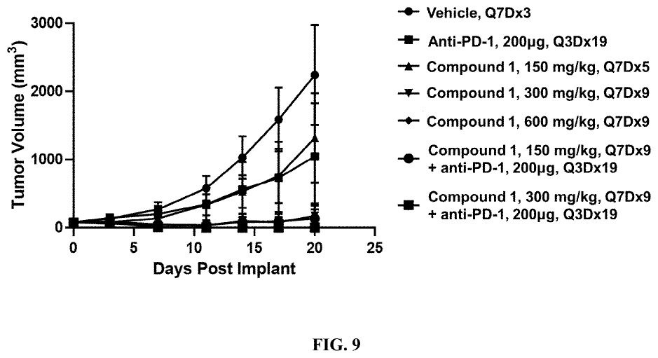

In another study, Compound 1 was administered to mice bearing MT245 tumors at 150, 300, or 600 mg/kg, once per week (Q7D). Mice receiving anti-PD-1 were given intraperitoneal injections of 200 μg of anti-PD-1 (clone BE0146), once every three days (Q3D). As shown in FIG. 9, combination therapy significantly increased efficacy for mice receiving Compound 1. Mice that received 150 mg/kg of Compound 1 in combination with anti-PD-1 demonstrated 97.3% TGI at day 20, with an extended median survival time of >157.00 days, while the group that received 300 mg/kg of compound 1 plus anti-PD-1 achieved 99.9% regression and extended median survival time to >157.0 days, compared to 19.3 days for vehicle, 24.9 days for anti-PD-1 monotherapy, 25.4 days for Compound 1 monotherapy at 150 mg/kg, and 41.2 days for Compound 1 monotherapy at 300 mg/kg.

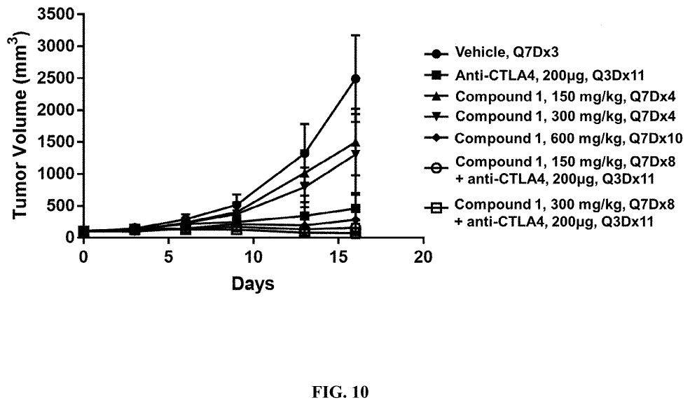

In an additional study, Compound 1 was administered to mice bearing MT373 tumors at 150, 300, or 600 mg/kg, once per week (Q7D). Some treatment groups received 200 μg of an anti-CTLA4 antibody (clone 9H10) via intraperitoneal injection, once every three days (Q3D). Mice in the 150 mg/kg combination group demonstrated an increase in median survival from 20.27 days with single agent to 38.84 day in combination with anti-CTLA4 (FIG. 10). The 300 mg/kg group had an increase in median survival from 31.99 days with single agent to >140 days in combination with anti-CTLA4, with 50% of that group surviving until termination of the study at day 140.

These results demonstrate that combination therapy with a p53 reactivating compound and an immune checkpoint inhibitor can result in synergistic effects, which can include tumor regression and an increase in median survival time. These results also suggest that reactivation of p53 can potentiate an anti-cancer immune response. Further, these results demonstrate that syngeneic mouse cancer models utilizing cell lines generated from Hupki-Y220C mice can be used to study anti-cancer combination therapies, including combination therapies that include immunomodulatory agents, such as immune checkpoint inhibitors.

### Example 8

### Profiling Tumor-Infiltrating Leukocyte Subsets and Cytokine Production in a Syngeneic Mouse Cancer Model

The effects of Compound 1 administration on anti-cancer immune responses was evaluated in a mouse syngeneic tumor model utilizing the MT373 cell line generated in example 2. Mice bearing MT373 tumors were treated with 75, 150, or 300 mg/kg of Compound 1 twice daily once per week for either 1, 2, or 3 doses (2Q7Dx1, 2Q7Dx2, or 2Q7Dx3). Tumors were harvested 72 hrs after the final dose, and tumor infiltrating leukocyte subsets were quantified by flow cytometry.

Tumors were dissected, and dissociated with a murine tumor dissociation kit and benchtop tissue dissociator. Re-suspended samples were washed, and the cells filtered through a cell strainer with 10 mL wash buffer to get single-cell suspensions. Tubes were spun by centrifuge at 300 g for 5 minutes, supernatant discarded, and cells re-suspended with 5 mL wash buffer. Cell concentration was adjusted to 1×10⁻⁶ cells per tube or per sample. Cells were Fc-blocked, stained for surface and intracellular markers, and analyzed via flow cytometry. Antibodies used are provided in TABLE 3.

TABLE 3
antibodies used in flow cytometry

| Target | Fluorochrome | Clone | Isotypes |
|---|---|---|---|
| CD45 | BUV661 | 30-F11 | Rat IgG2b, κ |
| CD3 | BUV395 | 145-2C11 | Armenian Hamster IgG1, κ |
| CD4 | BV421 | GK1.5 | Rat IgG2b, κ |
| CD8 | PE-eFluor610 | 53-6.7 | Rat IgG2a, κ |
| FoxP3 | PE | FJK-16S | Rat IgG2a, κ |
| CD335 | BV605 | 29A1.4 | Rat IgG2a, κ |
| CD11b | PE-Cy7 | M1/70 | Rat IgG2b, κ |
| F4/80 | BV510 | BM8 | Rat IgG2a, κ |
| CD206 | Percp-cy5.5 | C068C2 | Rat IgG2a, κ |
| I-A/I-E | BB515 | 2G9 | Rat IgG2a, κ |
| Ly-6G | BV785 | 1A8 | Rat IgG2a, κ |
| Ly-6C | APC | HK1.4 | Rat IgG2c, κ |
| L/D | efluo780 | NA | NA |

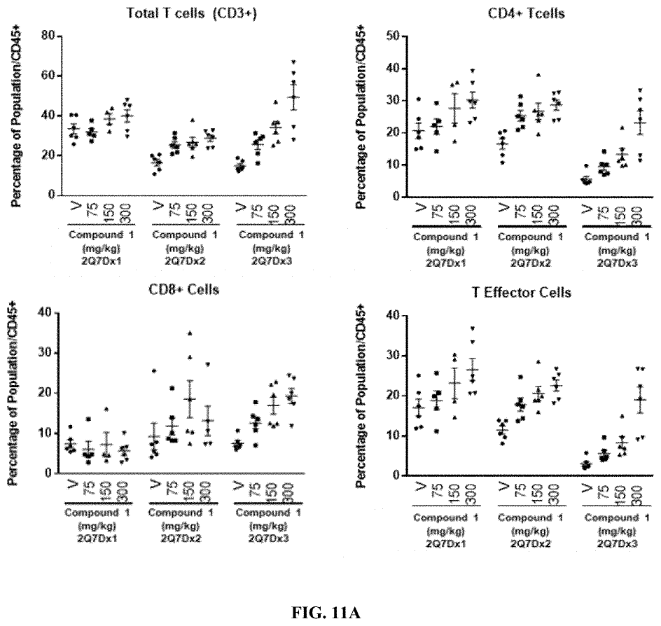

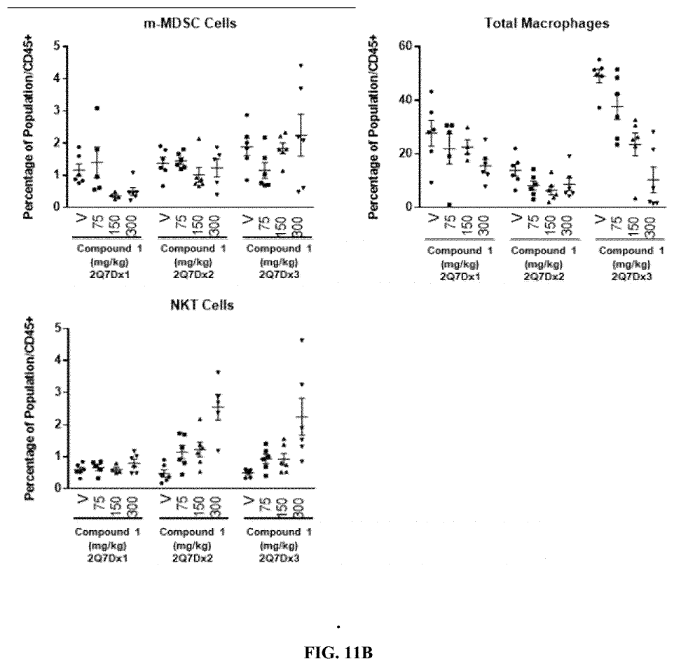

Treatment with Compound 1 elicited dose-proportional increases in total T cells (CD3+), CD4+ T cells, CD8+ T cells, T effector cells, and NKT cells (FIG. 11A and FIG. 11B). Decreases in total T cells in the vehicle treated groups over time was consistent with exclusion of T cells as tumor growth progressed. Dose-proportional decreases in suppressor cells were observed, for example, decreases in macrophages across all time points, and in m-MDSC cells at the earliest time point (FIG. 11B). These results demonstrate the reactivation of p53 with Compound 1 promotes an influx of T cells and NKT cells into a tumor, suggesting that p53 reactivation can increase an anti-cancer immune response and convert a tumor from immunologically “cold” to immunologically “hot.” These results also demonstrate that syngeneic mouse cancer models utilizing cell lines generated from Hupki-Y220C mice can be used to study the effects of candidate therapeutics on tumor-infiltrating leukocyte subsets.

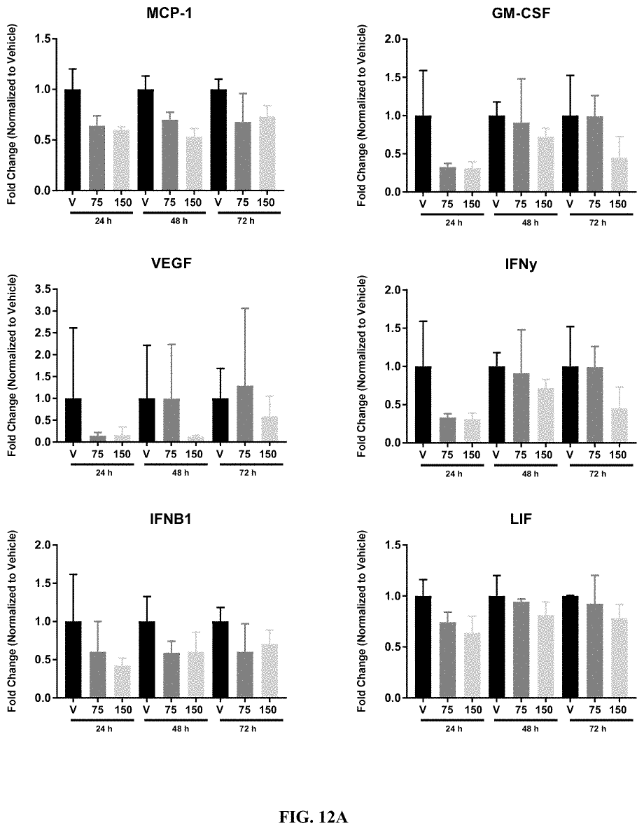

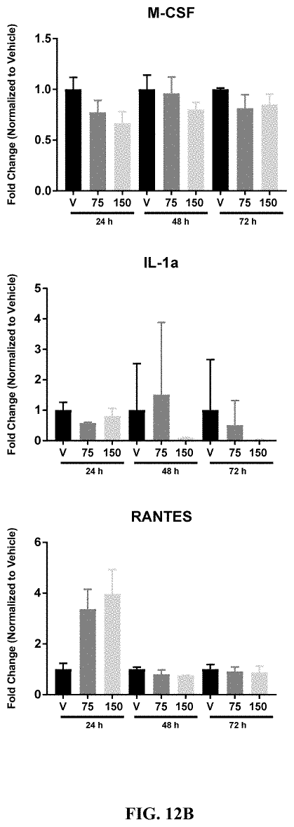

The effects of Compound 1 administration on cytokine production were also evaluated. Mice bearing MT373 tumors were treated with 75 or 150 mg/kg of Compound 1, twice in one day (BIDx1). Tumors were harvested at 24, 48, or 72 hours after dosing. Lysis buffer was added to tumor samples, which were homogenized using a bead mill. Homogenized samples were spun by centrifuge for 30 minutes at 20,817×g, and the supernatant transferred to a 1.5 mL tube. Total protein in samples was quantified using BCA. Samples were diluted to a protein concentration of 1 μg/mL. Cytokine concentrations in samples were determined using 44-plex cytokine panel. Results are shown in FIG. 12A, FIG. 12B, and TABLE 4 relative to vehicle control. Results are the average of n=3 per group, ±standard deviation. Mice that received

Compound 1 showed decreases in some cytokines relative to vehicle control. For example, 72 hours after treatment, mice that received Compound 1 exhibited reduced levels of IL1a (49.22% for 75mg/kg, 96.96% for 150 mg/kg), MCP-1 (32.15% for 75mg/kg, 26.90% for 150 mg/kg), and IFNB-1 (39.68% for 75 mg/kg, 29.51% for 150 mg/kg), compared to vehicle control. An increase in RANTES was seen at both dose levels at 24 h (3.37-fold for 75 mg/kg and 3.96-fold for 150 mg/kg). These results demonstrate that mouse syngeneic cancer models utilizing cell lines generated from Hupki-Y220C mice can be used to study in vivo cytokine profiles within tumors, including the impact of candidate therapeutics on cytokine profiles.

TABLE 4
changes in intra-tumor cytokine levels in mice treated with Compound 1
relative to vehicle control per timepoint (vehicle control set to 1.00). Increases are shown as fold
increases relative to vehicle control. Decreases are shown as percent reduction compared to
vehicle control, indicated by “%”. Averages are provided for 3 mice per group, ± St.Dev. N/A
signifies that sample measurements were out of range.
TABLE 4

| | Compound 1 75 mg/kg BIDx1 | | | Compound 1 150 mg/kg BIDx1 | | |
|---|---|---|---|---|---|---|
| | 24 h | 48 h | 72 h | 24 h | 48 h | 72 h |
| Eotaxin | 1.50 ± 0.05 | 1.03 ± 0.02 | 1.02 ± 0.08 | 1.43 ± 0.06 | 2.62% ± 4.13 | 1.01 ± 0.04 |
| G-CSF | 68.40% ± 24.21 | 4.49 ± 4.60 | 1.01 ± 0.61 | 64.22% ± 55.95 | 1.55 ± 1.65 | 1.53 ± 1.53 |
| GM-CSF | 67.34% ± 4.70 | 9.20% ± 57.29 | 1.14% ± 17.17 | 68.67% ± 8.01 | 27.78% ± 11.32 | 55.11% ± 27.67 |
| IFNγ | 51.31% ± 7.82 | 57.50% ± 41.62 | 9.06% ± 42.82 | 51.22% ± 13.29 | 36.62% ± 6.26 | 21.15% ± 25.96 |
| IL-1α | 41.77% ± 1.57 | 1.51 ± 2.37 | 49.22% ± 80.96 | 18.70% ± 24.80 | 91.13% ± 0.78 | 96.96% ± 0.92 |
| IL-1β | 18.38% ± 18.33 | 0.97% ± 32.49 | 1.14 ± 0.37 | 21.23% ± 16.26 | 1.08% ± 3.29 | 5.04% ± 8.43 |
| IL-2 | 31.69% ± 9.41 | 10.27% ± 37.27 | 1.24 ± 1.01 | 26.48% ± 25.45 | 11.31% ± 5.22 | 24.64% ± 23.35 |
| IL-3 | 78.81% ± 8.42 | 34.39% ± 30.58 | 59.78% ± 43.77 | 79.86% ± 8.16 | 55.73% ± 37.77 | 83.37% ± 1.59 |
| IL-4 | 12.00% ± 15.66 | 1.33 ± 0.22 | 16.54% ± 10.76 | 43.51% ± 8.15 | 18.19% ± 21.80 | 11.15% ± 6.25 |
| IL-5 | 29.58% ± 14.40 | 1.27 ± 0.98 | 19.96% ± 38.78 | 66.05% ± 14.40 | 32.87% ± 42.77 | 15.45% ± 3.57 |
| IL-6 | 68.42% ± 4.74 | 40.40% ± 17.23 | 2.03 ± 0.19 | 67.82% ± 10.22 | 59.46% ± 4.83 | 1.37 ± 0.49 |
| IL-7 | 13.05% ± 11.72 | 7.90% ± 33.82 | 18.49% ± 17.45 | 14.81% ± 7.19 | 10.95% ± 18.27 | 15.41% ± 19.95 |
| IL-9 | 6.63% ± 23.72 | 16.16% ± 22.71 | 5.19% ± 11.23 | 22.52% ± 10.43 | 18.58% ± 11.45 | 10.21% ± 7.96 |
| IL-10 | 34.82% ± 14.66 | 3.00% ± 59.83 | 1.18 ± 0.46 | 22.14% ± 16.70 | 13.04% ± 21.84 | 35.31% ± 6.37 |
| IL-12 (p40) | 18.87% ± 11.08 | 1.00 ± 0.52 | 2.05% ± 89.97 | 14.45% ± 27.49 | 54.23% ± 1.88 | 7.09% ± 7.39 |
| IL-12 (p70) | 52.75 ± 20.43 | 1.25 ± 0.91 | 73.18 ± 12.62 | 42.68 ± 14.25 | 44.97 ± 46.18 | 2.62 ± 4.04 |
| IL-13 | 49.63% ± 3.23 | 17.78% ± 19.75 | 6.25% ± 8.45 | 57.53% ± 9.84 | 15.56% ± 0.00 | 9.38% ± 2.71 |
| IL-15 | 23.94% ± 3.95 | 5.96% ± 30.83 | 1.04 ± 0.05 | 22.92% ± 19.06 | 14.17% ± 8.19 | 11.47% ± 10.00 |
| IL-17 | 30.72% ± 6.87 | 7.05% ± 15.32 | 13.76% ± 17.20 | 22.62% ± 18.32 | 15.26% ± 4.81 | 7.00% ± 11.98 |
| IP-10 | 63.55% ± 10.07 | 38.22% ± 15.54 | 24.39% ± 12.04 | 59.33% ± 4.53 | 59.71% ± 2.73 | 33.98% ± 18.18 |
| KC | 49.97% ± 2.88 | 18.22% ± 9.72 | 6.56% ± 14.93 | 53.30% ± 6.01 | 39.77% ± 3.28 | 20.61% ± 13.70 |
| LIF | 25.67% ± 9.92 | 5.51% ± 2.52 | 7.52% ± 27.72 | 36.16% ± 16.29 | 18.65% ± 12.99 | 21.85% ± 13.57 |
| LIX | 79.72% ± 10.09 | 2.96 ± 3.78 | 31.26% ± 26.57 | 67.84% ± 21.65 | 2.36 ± 2.16 | 72.62% ± 19.25 |
| MCP-1 | 36.01% ± 9.92 | 29.99% ± 7.31 | 32.15% ± 28.15 | 40.13% ± 3.13 | 46.90% ± 8.21 | 26.90% ± 10.59 |
| M-CSF | 22.71% ± 11.64 | 3.90% ± 16.14 | 18.70% ± 13.39 | 33.55% ± 11.46 | 19.89% ± 6.90 | 15.12% ± 10.28 |
| MIG | N/A | N/A | N/A | N/A | N/A | N/A |
| MIP-la | 29.50% ± 16.88 | 16.29% ± 15.75 | 9.52% ± 21.85 | 9.87% ± 26.67 | 19.26% ± 3.54 | 5.39% ± 16.93 |
| MIP-1B | 21.01% ± 19.14 | 9.75% ± 11.97 | 0.88% ± 26.42 | 4.28% ± 25.36 | 15.72% ± 4.57 | 3.77% ± 19.04 |
| MIP-2 | 57.27% ± 4.22 | 26.86% ± 7.95 | 1.16 ± 0.50 | 54.37% ± 15.39 | 34.35% ± 5.87 | 21.49% ± 7.42 |
| RANTES | 3.37 ± 0.78 | 20.38% ± 16.82 | 8.80% ± 18.49 | 3.96 ± 0.97 | 25.23% ± 1.45 | 12.67% ± 26.18 |
| TNFα | 38.56% ± 13.40 | 18.05% ± 16.37 | 1.02 ± 0.26 | 28.26% ± 25.00 | 29.21% ± 4.41 | 11.95% ± 24.54 |
| VEGF | 85.75% ± 7.38 | 0.82% ± 124.14 | 1.29 ± 1.77 | 84.07% ± 18.58 | 88.31% ± 3.75 | 41.22% ± 45.89 |
| EPO | 29.48% ± 13.48 | 12.73% ± 25.44 | 8.40% ± 10.57 | 7.07% ± 40.37 | 7.76% ± 17.31 | 12.00% ± 7.99 |
| 6Ckine/Exodus 2 | 3.33 ± 2.27 | N/A | N/A | N/A | 1.47 ± 1.43 | N/A |
| Fractalkine | 13.01% ± 19.98 | 23.57% ± 6.00 | 23.50% ± 18.28 | 4.30% ± 28.14 | 1.12 ± 0.15 | 13.73% ± 11.84 |
| IFNβ-1 | 39.69% ± 39.80 | 40.77% ± 15.09 | 39.68% ± 36.83 | 57.70% ± 9.69 | 39.82% ± 25.60 | 29.51% ± 18.07 |
| IL-11 | 11.39% ± 35.71 | 38.57% ± 20.57 | 18.33% ± 62.76 | 1.01 ± 0.34 | 51.05% ± 11.42 | 15.59% ± 42.35 |
| IL-16 | 1.61% ± 44.74 | 7.31% ± 15.65 | 10.24% ± 15.49 | 1.17 ± 0.20 | 4.39% ± 14.03 | 1.16 ± 0.11 |
| IL-20 | 2.39% ± 12.91 | 1.18 ± 0.32 | 1.24 ± 0.32 | 1.81 ± 0.42 | 2.06 ± 1.40 | 9.63% ± 29.29 |
| MDC | N/A | 23.57% ± 49.15 | 88.96% ± 12.99 | N/A | 46.92% ± 26.02 | 89.81% ± 7.45 |
| MCP-5 | 1.05 ± 0.06 | 1.01 ± 0.07 | 6.49% ± 7.22 | 1.12 ± 0.07 | 2.02% ± 11.00 | 1.01 ± 0.06 |
| MIP-3α | 6.80% ± 13.95 | 10.94% ± 2.12 | 6.03% ± 5.98 | 1.01 ± 0.10 | 1.07 ± 0.23 | 14.46% ± 0.88 |
| MIP-3β | 14.06% ± 12.55 | 1.05 ± 0.06 | 1.03 ± 0.31 | 9.49% ± 3.62 | 1.02 ± 0.17 | 1.10 ± 0.15 |
| TARC | 1.02 ± 1.04 | 1.23 ± 0.87 | 34.71% ± 52.51 | 67.13% ± 10.92 | 52.42% ± 21.81 | 48.38% ± 12.02 |
| TIMP-1 | 2.50% ± 4.21 | 2.56% ± 1.28 | 0.08% ± 2.46 | 1.45% ± 1.41 | 1.39% ± 1.44 | 1.00 ± 0.012 |

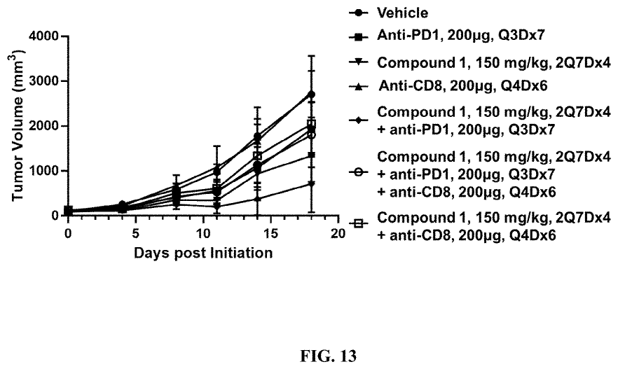

To study the role of CD8+ T cells in the anti-cancer immune response after p53 reactivation, mice bearing MT373 tumors were treated with Compound 1 and anti-PD-1 antibody (clone BE0146), with or without depletion of CD8+ T cells by treatment with an anti-CD8 antibody (clone 2.43). Anti-CD8 antibody was administered beginning three days prior to the first doses of Compound 1 and anti-PD-1. Anti-CD8 antibody was administered intraperitoneally at 200 μg/mouse every 4 days. Compound 1 was administered at 150 mg/kg, twice daily, once per week. Anti-PD-1 antibody was administered at 200m/mouse every 3 days. Results for 2Q7D administration are presented in FIG. 13. All tested dosing regimens were well tolerated by the mice across the course of study.

Single agent Compound 1 administration resulted in 52% tumor growth inhibition (TGI). The combination of Compound 1 and anti-PD-1 exhibited increased efficacy, with 76% TGI. However, when CD8+ T cells were depleted, single agent Compound 1 administration resulted in only 27% TGI, and the combination of Compound 1 and anti-PD-1 also exhibited a decrease in efficacy to 36% TGI. These results suggest that CD8+ T cells contribute to the anti-cancer immune response upon treatment with Compound 1 or a combination of Compound 1 and immune checkpoint inhibitor. These results also demonstrate that mouse syngeneic cancer models utilizing cell lines generated from Hupki-Y220C mice can be used to study the roles of particular cell subsets in cancer, for example, how cell subsets contribute to cancer progression or treatment efficacy.

### Example 9

### Nucleotide Sequence of the Top Strand of an Example Vector that was Used to Make the Y220C HUPKI p53

Features and locations are listed below. Amp: 576-1433; 5′arm: 2661-5739; E2: 5259-5341; E3: 5625-5646; hTP53 E4-E9: 5740-8477; hE4: 5740-6018; hE5: 6776-6959; hE6: 7041-7153; Y220C: 7139-7141; hE7: 7722-7831; hE8: 8175-8311; hE9: 8404-8477; LoxP: 8889-8922; Neo Cassette: 8960-12,718; LoxP: 12,721-12,754; 3′arm: 12,768-17,516; E10: 13,216-13,322; E11: 13,910-14,433; DTA Cassette: 17,568-19,003.

SEQ ID NO: 11

ttaatattttgttaaaattcgcgttaaatttttgttaaatcagctcattttt
taaccaataggccgaaatcggcaaaatcccttataaatcaaaagaatag
accgagatagggttgagtgttgttccagtttggaacaagagtccactattaaa
gaacgtggactccaacgtcaaagggcgaaaaaccgtctatc
agggcgatggcccactacgtgaaccatcaccctaatcaagttttttggggtc
gaggtgccgtaaagcactaaatcggaaccctaaagggagcc
cccgatttagagcttgacggggaaagccggcgaacgtggcgagaaaggaagg
gaagaaagcgaaaggagcgggcgctagggcgctgg
caagtgtagcggtcacgctgcgcgtaaccaccacacccgccgcgcttaatgc
gccgctacagggcgcgtcaggtggcacttttcggggaaa
tgtgcgcggaacccctatttgtttatttttctaaatacattcaaatatg
tatccgctcatgagacaataaccctgataaatgcttcaataatattgaaaa
aggaagagtatgagtattcaacatttccgtgtcgcccttattcccttat
tgcggcattttgccttcctgttatgctcacccagaaacgctggtgaaa
gtaaaagatgctgaagatcagttgggtgcacgagtgggttacatcgaact
ggatctcaacagcggtaagatccttgagagttttcgccccgaag
aacgttttccaatgatgagcacttttaaagttctgctatgtggcgcggta
ttatcccgtattgacgccgggcaagagcaactcggtcgccgcatac
actattctcagaatgacttggttgagtactcaccagtcacagaaaagca
tcttacggatggcatgacagtaagagaattatgcagtgctgccata
accatgagtgataacactgcggccaacttacttctgacaacgatcggag
gaccgaaggagctaaccgcttttttgcacaacatgggggatcat
gtaactcgccttgatcgttgggaaccggagctgaatgaagccataccaa
acgacgagcgtgacaccacgatgcctgtagcaatggcaacaac
gttgcgcaaactattaactggcgaactacttactctagcttcccggcaa
caattaatagactggatggaggcggataaagttgcaggaccacttc
tgcgctcggcccttccggctggctggtttattgctgataaatctggagc
cggtgagcgtgggtctcgcggtatcattgcagcactggggccaga
tggtaagccctcccgtatcgtagttatctacacgacggggagtcaggcaa
ctatggatgaacgaaatagacagatcgctgagataggtgcctc
actgattaagcattggtaactgtcagaccaagtttactcatatatactt
tagattgatttaaaacttcatttttaatttaaaaggatctaggtgaagatcc
tttttgataatctcatgaccaaaatcccttaacgtgagttttcgttcca
ctgagcgtcagaccccgtagaaaagatcaaaggatcttcttgagatcct
ttttttctgcgcgtaatctgctgcttgcaaacaaaaaaaccaccgctac
cagcggtggtttgtttgccggatcaagagctaccaactctttttccga
aggtaactggcttcagcagagcgcagataccaaatactgttcttctagtg
tagccgtagttaggccaccacttcaagaactctgtagcaccgcct
acatacctcgctctgctaatcctgttaccagtggctgctgccagtggcga
taagtcgtgtcttaccgggttggactcaagacgatagttaccggat
aaggcgcagcggtcgggctgaacggggggttcgtgcacacagcccagctt
ggagcgaacgacctacaccgaactgagatacctacagcgt
gagctatgagaaagcgccacgcttcccgaagggagaaaggcggacaggta
tccggtaagcggcagggtcggaacaggagagcgcacga
gggagcttccagggggaaacgcctggtatattatagtcctgtcgggttt
cgccacctctgacttgagcgtcgatttttgtgatgctcgtcagggg
ggcggagcctatggaaaaacgccagcaacgcggcctttttacggttcct
ggccttttgctggcatttgctcacatgttattcctgcgttatcccct
gattctgtggataaccgtattaccgcctttgagtgagctgataccgctc
gccgcagccgaacgaccgagcgcagcgagtcagtgagcgagga
agcggaagagcgcccaatacgcaaaccgcctctccccgcgcgttggccg
attcattaatgcagctggcacgacaggtttcccgactggaaa
gcgggcagtgagcgcaacgcaattaatgtgagttagctcactcattag
gcaccccaggattacactttatgcttccggctcgtatgttgtgtgga
attgtgagcggataacaatttcacacaggaaacagctatgaccatgat
tacgccaagctcgaaattaaccctcactaaagggaacaaaagctg
gtacgcggccgcaagtgataccagacacagccttcttattatcttag
aaccaacaatatatcggtccttggcccccacccccatgcccatttttc
caatgctgggtgttgaacccggagccttagacataacacacgaactc
tgcagctgagctaccctgccagctccgaaagatttgtatactaagttc
tatgaaacattaaggtcgttttattcagggtaaggtgtggcttctgg
cttccctgggtccagtcattgatggtggtgggtagtactgttcgttccatt
ccgtttggggttttgattgacaagccttgcacctttccaactgttct
acctcaagagccaaagataaagggtaaaagattcccttcccttatccctgt
caatagcagcctgcctagcttcctcaggatcaaatgagatgagcccct
gagaagagcaaggcccgctgggcctggaaggccagccctggtt
gtactcaaacctctcgagtctattgcattcccagccaacattttctta
cacatccagcctctgtggatactgtgaccctcctgatctggttcttgtga
aaagtttcatattggcaactgttcttaaggaaacactgaaaacctaatt
actactaacgacctggaagatagagcaggaagacctcctattgtcag
tgtggctatgtctccaagactgagacattgggccgccatcccagcatg
tgcgcgcgcgcgcgcgcacacacacacacacacacacagtattc
actaagcaagagttccttgacagggtgaactggcattaacgggtagtg
gttagcgccacagaagcactgctagctcagaacaaatgaggtgct
catacccacagggctgagctcttacatgagtgtgtactgttgggcgtg
gggtggggctggacccagggcttcacagatgagctccgcccaag
gccaaatctcaaaaaatgaaaaggtcaaaaggtaaccattcagatagat
accggagatatgatatttacaaaaaatatgataattaaatgctaca
gttttaattatgaatctgaagcagctctgggtctccctgacccattcca
gggccagccacctcattactcagatgtaggggctatataatacccag
tcattgattcttcatctagctgattgcaagagaactgtgcctaagagcc
tgtgacgcactagattgtttgggtggccatattaataaagagtcatt
cacctgcccaggggcagatgtgaccatcgagacagatgaaatagcccat
atggaaaggtttctcaaactcttaaataaatgagaacttgattgtt
ctacatacaagtgaatatattaaatgttgagattatagtcagtattaac
agtaattattctgaaatgcttgcattgctgttagaaaggaaacatgatgt
cccagggggttggccgaactcaatccccagaacccacttcttataagtt
gtcatctgacctccatacgcatgtcttggcacattaaataaggcac
aataaataagtaaatgtctaagtaaggaagataatagctccgtggttag
taacaagagaaaatgagtgagagagtatcatacggcttaacctctg
aaggaggtggtggtggtgtggctggctggagattggctggctgtgactg
tctccgaggagctatggcatacaagataaggaagagcctacct
catagcctagggagaacagaaactgtgactttgctcttgtagaggtaac
cacactgattcagccaggaggaagtaagtgctccctagagccctt
ggggaagaaggcagtaggaaacctgctgaatcttcaggaatttgtaag
gcgctggggacctgtccctagggggcagatgagacactgatgg
gcgtacttagagatttgccatgaagtgggtttgaagaatggagctgtg
tgtgaaatggtggatggggggggggagtccctccccagagggaa
gggaagagagatgagatgtagggtgcagatgtaggggggcttggggcta
gaagtacctccctgattacctgttccttgaagcagtgtgtggtt
cgagaagctgataggaagccaggccaaggcttgagcagcctgaataaa
agacggaagagctgccccattcctgcttctctggaaatggtgtc
cctcacggccatcttgggtcctgacttcttctcaaaggagcctggccga
cttcttggatacttgtaactttgcgattcccaccctcgcataagtttc
ctgaaataatgactctgaaactcaaaatatatttacaaacacctcggct
gtagattggtttgttctctgacttgcttataacttaatatccctcttattct
aacctaagttctgccacgtggttggttacctctgctcagcccccggctt
ctgtcctccatgttcctgggggaatccctacactttcagaatttaatttc
cctactggatgtcccaccttctttttattctaccctttcctataagcca
taggggtttgtttgtttgtatgttttttaattgacaagttatgcatccatacagt
acacaatctcttctctctacagatgactgccatggaggagtcacagtcg
gatatcagcctcgagctccctctgagccaggagacattttcaggct
tatggaaactgtgagtggatattttggggcccttaagatacatcccgcc
atacctgtatcctccccttgcctgagagaaacaaaaacagtagtgt
tcaaacatggtatggtgttgggtgtctgtaaatcctgcggggcggggtg
gcggggggggggggggactgcagggtctcagaagtttgaggt
catcattgactacatagcaagttggaggccagcctgggataagtgagat
tctgtcttcaaaaaatggaaggaaatcaggaactaactctctgctc
ttgttttccagacttcctccagaagatatcctggtaaggcccagagcag
aaagggacttgggattggtgttgggctggtaggctgagaacaca
gtcctgagggttcttattgtcccatccacagtcccccttgccgtcccaa
gcaatggatgatttgatgctgtccccggacgatattgaacaatggtt
cactgaagacccaggtccagatgaagctcccagaatgccagaggctgct
ccccccgtggcccctgcaccagcagctcctacaccggcggc
ccctgcaccagccccctcctggcccctgtcatcttctgtcccttcccag
aaaacctaccagggcagctacggtttccgtctgggcttcttgcattc
tgggacagccaagtctgtgacttgcacggtcagttgccctgaggggctg
gcttccatgagacttcaatgcctggccgtatccccctgcatttcttt
tgtttggaactttgggattcctcttcaccattggcttcctgtcagtgtt
tttttatagtttacccacttaatgtgtgatctctgactcctgtcccaaagttg
aatattccccccttgaatttgggatttatccatcccatcacaccctcag
catctctcctggggatgcagaacttttctttttcttcatccacgtgtattc
cttggatttgaaaataagctcctgaccaggcttggtggctcacacctgc
aatcccagcactctcaaagaggccaaggcaggcagatcacctg
agcccaggagttcaagaccagcctgggtaacatgatgaaacctcgtctc
tacaaaaaaatacaaaaaattagccaggcatggtggtgcacac
ctatagtcccagccacttaggaggctgaggtgggaagatcacttgaggc
caggagatggaggctgcagtgagctgtgatcacaccactgtgc
tccagcctgagtgacagagcaagaccctatctcaaaaaaaaaaaaaaaa
aagaaaagctcctgaggtgtagacgccaactctctctagctcg
ctagtgggttgcaggaggtgcttacgcatgtttgtttattgctgccgtct
tccagttgctttatctgttcacttgtgccctgactttcaactctgtctcct
tcctcttcctacagtactcccctgccctcaacaagatgttttgccaactg
gccaagacctgccctgtgcagctgtgggttgattccacacccccgc
ccggcacccgcgtccgcgccatggccatctacaagcagtcacagcacatga
cggaggttgtgaggcgctgcccccaccatgagcgctgctc
agatagcgatggtgagcagctggggctggagagacgacagggctggttgc
ccagggtccccaggcctctgattcctcactgattgctcttag
gtctggcccctcctcagcatcttatccgagtggaaggaaatttgcgtgtgg
agtatttggatgacagaaacacttttcgacatagtgtggtggtgc
cctgtgagccgcctgaggtctggtttgcaactggggtctctgggaggag
gggttaagggtggttgtcagtggccctccaggtgagcagtagg
ggggctttctcctgctgcttatttgacctccctataaccccatgagatg
tgcaaagtaaatgggtttaactattgcacagttgaaaaaactgaagctt
acagaggctaagggcctcccctgcttggctgggcgcagtggctcatgc
ctgtaatcccagcactttgggaggccaaggcaggcggatcacg
aggttgggagatcgagaccatcctggctaacggtgaaaccccgtctcta
ctgaaaaatacaaaaaaaaattagccgggcgtggtgctgggca
cctgtagtcccagctactcgggaggctgaggaaggagaatggcgtgaa
cctgggcggtggagcttgcagtgagctgagatcacgccactgc
actccagcctgggcgacagagcgagattccatctcaaaaaaaaaaaaa
aaaggcctcccctgcttgccacaggtctccccaaggcgcactg
gcctcatcttgggcctgtgttatctcctaggttggctctgactgtacc
accatccactacaactacatgtgtaacagttcctgcatgggcggcatga
accggaggcccatcctcaccatcatcacactggaagactccaggtcagg
agccacttgccaccctgcacactggcctgctgtgccccagcct
ctgcttgcctctgacccctgggcccacctcttaccgatttcttccatac
tactacccatccacctctcatcacatccccggcggggaatctccttac
tgctcccactcagttttcttttctctggattgggacctcttaacctgtg
gcttctcctccacctacctggagctggagcttaggctccagaaaggac
aagggtggttgggagtagatggagcctggttttttaaatgggacaggt
aggacctgatttccttactgcctcttgcttctcttttcctatcctgagtag
tggtaatctactgggacggaacagattgaggtgcgtgtttgtgcctgtc
ctgggagagaccggcgcacagaggaagagaatctccgcaaga
aaggggagcctcaccacgagctgcccccagggagcactaagcgaggta
agcaagcaggacaagaagcggtggaggagaccaagggtg
cagttatgcctcagattcacttttatcacattccttgcctattcctagc
actgcccaacaacaccagctcctctccccagccaaagaagaaacca
ctggatggagaatatttcacccttcaggtaccaaggctggagagtcgca
tgccagagacaagctgtacccattattgcctctgtctctcgcatgt
ataaaatagtgttattagcaggttgccaggtctttttcagtggattatc
tctagccgtgacattagcttgagagctcatgctctgaggctgtgcctcc
tccgacagtggttctcagtgtaactaacttgacaacaccaacttacacca
taagacaggtgctcctccactggggatgggaacatgtcctagga
aactcaccataaattgaaaataacacacgacaaaaatgtgtttagaggca
ggcctggtggcacctgcctgtaattacagcactggtcgacctgc
agccaagctatcgaattcctgcagcccaattccgatcatattcaataacc
cttaatataacttcgtataatgtatgctatacgaagttattaggtctga
agaggagtttacgtccagccaagctagctccatgggccaggcaaatatcc
cttaccagcctcacagagacctcccccaccccccgcaaccct
agagttatttactagtgagggacaagtggacaatggtgctgttgtgggcc
ccaccctgtgtcccctgtgcccacagtggtcactctgcttggca
ggcaggtgttgcaggctggctgctccaggccctggcaggaggtactgaag
gacctggtaggctcagatgccctggatgccaaggcactgct
ggagtacttccaaccggtcagccagtggctggaagagcagaatcagcgga
atggcgaagtcctaggctggccagagaatcagtggcgtcc
accgttacccgacaactatccagagggcattggtaaagctctgagtgagg
gtggactgggaccaagagaagtcctggcctctggcctctggc
ttctgggtcaaagcctcagcatcctggtcactttgctgccagctgag
ccccagtgtcattgcttcagtgccaagccacccctgggctcatcctca
gggccctaagcagaaatgggtatgtattctctcagggtcctagaga
cagtgtgcccaagcctgagggcccttggggtcaggctggctggca
cattgctctatgaggtcacactgcaggcttggctcttattggccggtg
atgggagcttcagggctctgattcctgcggccatgcccaagaagaa
gaggaaggtgtccaatttactgaccgtacaccaaaatttgcctgcatt
accggtcgatgcaacgagtgatgaggttcgcaagaacctgatggac
atgttcagggatcgccaggcgttttctgagcatacctggaaaatgctt
ctgtccgtttgccggtcgtgggcggcatggtgcaagttgaataaccg
gaaatggtttcccgcagaacctgaagatgttcgcgattatcttctata
tcttcaggcgcgcggtctggcagtaaaaactatccagcaacatttggg
ccagctaaacatgcttcatcgtcggtccgggctgccacgaccaagtga
cagcaatgctgtttcactggttatgcggcggatccgaaaagaaaa
cgttgatgccggtgaacgtgcaaaacaggctctagcgttcgaacgcac
tgatttcgaccaggttcgttcactcatggaaaatagcgatcgctgc
caggatatacgtaatctggcatttctggggattgcttataacaccctg
ttacgtatagccgaaattgccaggatcagggttaaagatatctcacgta
ctgacggtgggagaatgttaatccatattggcagaacgaaaacgctggtt
agcaccgcaggtgtagagaaggcacttagcctgggggtaact
aaactggtcgagcgatggatttccgtctctggtgtagctgatgatccgaa
taactacctgttttgccgggtcagaaaaaatggtgttgccgcgcc
atctgccaccagccagctatcaactcgcgccctggaagggatttttgaag
caactcatcgattgatttacggcgctaaggtaaatataaaattttta
agtgtataatgtgttaaactactgattctaattgtttgtgtattttagga
tgactctggtcagagatacctggcctggtctggacacagtgcccgtgtc
ggagccgcgcgagatatggcccgcgctggagtttcaataccggagatcat
gcaagctggtggctggaccaatgtaaatattgtcatgaactat
atccgtaacctggatagtgaaacaggggcaatggtgcgcctgctggaaga
tggcgattagccattaacgcgtaaatgattgctataattatttga
tatttatggtgacatatgagaaaggatttcaacatcgacggaaaatatgt
agtgctgtctgtaagcactaatattcagtcgccagccgtcattgtca
ctgtaaagctgagcggcaataaaaagacagaataaaacgcacgggtgttg
ggtcgtttgttcggtcgagctcgcgaagctagcttggctgcag
gtcgtcgaaattctaccgggtaggggaggcgcttttcccaaggcagtct
ggagcatgcgctttagcagccccgctgggcacttggcgctacac
aagtggcctctggcctcgcacacattccacatccaccggtaggcgccaac
cggctccgttattggtggccccttcgcgccaccttctactcctc
ccctagtcaggaagttcccccccgccccgcagctcgcgtcgtgcaggacg
tgacaaatggaagtagcacgtctcactagtctcgtgcagatg
gacagcaccgctgagcaatggaagcgggtaggcctttggggcagcggcc
aatagcagattgctccttcgattctgggctcagaggctggg
aaggggtgggtccgggggcgggctcaggggcgggctcaggggcggggcgg
gcgcccgaaggtcctccggaggcccggcattctgcac
gcttcaaaagcgcacgtctgccgcgctgttctcctcttcctcatctccgg
gcctttcgacctgcagcctgttgacaattaatcatcggcatagtata
tcggcatagtataatacgacaaggtgaggaactaaaccatgggatcggccat
tgaacaagatggattgcacgcaggttctccggccgcttggg
tggagaggctattcggctatgactgggcacaacagacaatcggctgctctg
atgccgccgtgttccggctgtcagcgcaggggcgcccggtt
ctttttgtcaagaccgacctgtccggtgccctgaatgaactgcaggacgagg
cagcgcggctatcgtggctggccacgacgggcgttccttgc
gcagctgtgctcgacgttgtcactgaagcgggaagggactggctgctattgg
gcgaagtgccggggcaggatctcctgtcatctcaccttgct
cctgccgagaaagtatccatcatggctgatgcaatgcggcggctgcatacg
cttgatccggctacctgcccattcgaccaccaagcgaaacat
cgcatcgagcgagcacgtactcggatggaagccggtcttgtcgatcaggat
gatctggacgaagagcatcaggggctcgcgccagccgaa
ctgttcgccaggctcaaggcgcgcatgcccgacggcgatgatctcgtcgtg
acccatggcgatgcctgcttgccgaatatcatggtggaaaat
ggccgcttttctggattcatcgactgtggccggctgggtgtggcggaccgc
tatcaggacatagcgttggctacccgtgatattgctgaagagc
ttggcggcgaatgggctgaccgcttcctcgtgctttacggtatcgccgctc
ccgattcgcagcgcatcgccttctatcgccttcttgacgagttctt
ctgaggggatcaattctctagagctcgctgatcagcctcgactgtgccttct
agttgccagccatctgttgtttgcccctcccccgtgccttccttg
accctggaaggtgccactcccactgtcattcctaataaaatgaggaaattgca
tcgcattgtctgagtaggtgtcattctattctggggggtggg
gtggggcaggacagcaagggggaggattgggaagacaatagcaggcatgctg
gggatgcggtgggctctatggcttctgaggcggaaag
aaccagctggggctcgactagagcttgcggaacccttaatataacttcgtat
aatgtatgctatacgaagttattaggtccctcgagaagagggt
ggttcaaagctggcaagaaaaagtaacccaaggaaaggacattggggtttaa
gggtataactcagttccaggatacttgccaagtgtgtgcca
ggccccggattaggtccccagcggctcacggtgcattagtccacctaaccc
acaactggactctacagctcagctcccggttgccctgttaag
cgtctgttccctctaactgaaaagatccaagcttgttgtacacgttctact
gaatgcctgttactttcacaccatcttattaaagatgatctccccccc
ccccaaaaaaaaaaacaaaacaaaacaaaacaaaaacctgtaagtggagcca
gcttaagttgggaaccaactttcagaaagaaagttgttaa
aatcgtgaaagtggttgtgtgaccttgtccagtgcttccatctcacttcatc
tctgctgcagatccgcgggcgtaaacgcttcgagatgttccggg
agctgaatgaggccttagagttaaaggatgcccatgctacagaggagtctg
gagacagcagggctcactccaggtaagtggcctggggcag
cgcctgcctgtggtgctctacccagacctccctccagctcagcattgtagtg
aaagataaaaaccccaccctgctagatgcttagggctgcac
cctacgagaactgacttcttgactttttaggctctgttaaggggtatgaggg
acaaggtatggtgtcatgctcctataatctcagcagtaaggaag
acaaagtcaggaggatttggggaagtttgaagccttcataaactatataaaa
ctttaggccagctagggctagctacaatagcaagaccctgtct
catggtgatggtgatgatggtggtggtggtgacagttgtgataataatggca
gtggtggtgatgatgatggtggtggtgatgatggtgatggtga
aagggaggataaactgattctcagaagtattccagtgtgttctgtgaatatc
cctacccatagtagaagccatcttaaattcctttttttcagcctcca
gcctagagccttccaagccttgatcaaggaggaaagcccaaactgctagctc
ccatcacttcatccctccccttttctgtcttcctatagctacctg
aagaccaagaagggccagtctacttcccgccataaaaaaacaatggtcaaga
aagtggggcctgactcagactgactgcctctgcatcccgt
ccccatcaccagcctccccctctccttgctgtcttatgacttcagggctgag
acacaatcctcccggtcccttctgctgccttttttaccttgtagcta
gggctcagccccctctctgagtagtggttcctggcccaagttggggaataggt
tgatagttgtcaggtctctgctggcccagcgaaattctatcc
agccagttgttggaccctggcacctacaatgaaatctcaccctaccccacac
cctgtaagattctatcttgggccctcatagggtccatatcctcc
agggcctactttccttccattctgcaaagcctgtctgcatttatccaccccc
caccctgtctccctcttttttatttttacccattttatatatcaatttcc
tattttacaataaaattttgttatcacttatatggttttgagaggttgatatc
agcataagctgtctgggcccccaggggcaggatgaatttgggagg
tacccacctgagtccaggcagtctgttggagtcaggggtggggaacttgggtt
ccagaagaggaacaaagccggggactgggtcagtctgt
gggctgcatgacaacaagagagcagggtgactccattcataacttgggaacc
aactgtccctcctccctcctgccaggactggcacatggtcc
ttacccacccctacttctagggttgggctcctgctgtcctctggggagcctct
caccaaggattaagggatttaaatgtctgatatagcaaacctg
agcctctggagtgaccatctgctccacaagaaaggatcagggtgcctggg
ttccacagggaagggggtggctgttcctggatgaagagaca
agtgggaggcccgccagctggggtcccaggaaactgggagcagttaaggtg
aggctaggggccttcccagcatccccaaactccgggcct
cacgccaggcaagtgaatgaatcgaagattgcactttgccaggaaatcatt
gaaagggcttcttggagtagggaggagagtcagagttggg
gagcctaacaccctctcacccacccccttcctcagtgctgctggctcccag
agagcgggactaggccaagctctccccatagcctgctgagc
cagtgccagtagggagagcagtcggtgagcacatggcttggccatggctctt
ggtaacagagagaggggtactgaggtccagaggcacttt
ggggccactgtcctctgcctgtcccaggcttaaagagccagtgagagtcat
tgccttgcccgagggtcctatacaagttgggagggagcagc
cccttagcctcaaaattttgtacttgtgagtaccaaatattttcgcagcag
actagggtctatcttataactgttgtgggaatgtgtgatgccagtca
aacccactacgcttacccctgtttttgtcccacagattactgagaactaaatc
caaagaactgtgtttaagggccacaaatttgagcccttatctc
aaaaagggaggaggggctaaaacaattgccaaatgaaaagctatctctgcat
ttgtgcctgtatggtaaacgttaagactgacagagaactact
caacaaaagctcctccacccaactcattattatttcagacagtaccttgct
aactaagtagcccaggctaccctcaacattggtctattatttagct
ttccaagtggagggagggatcgatgggctaccatacttccttaccttccccat
ttccctatcttttgtccttaattctgtccttgtacctcaagtcttca
gaaagcaggtccattttggtacctcaaatcccttctctgagagctgatatcaa
tggacaaaggataatgaggttatgcttctcctgcaagctcttct
atgtaacttcacatcagtcacttacctcccaggttccacttccgggctgttt
ccattgatgcatcaactaggcatcgatccataaagtacttgacatt
cacagggaccctatgtctgctatgatgtctgcccaagaggtaaatacctcag
gccttactctattgaggtctaaaatcaaacctcccaatctctaat
cttgtaactctaacctacttttacattttaggtcaatatattttttatttatg
tattttgagaaatggtggcgtgccagaccatgataagcaagacattgta
ccagcaattaaccccccacccccaactccatagctgggtggtgatggagcac
acctttaatcctggcacttgggacacagaggcaggcagag
ttcagcctagtaaacagagtgagttccagtccagccagggctatacagagaaac
cctgtctcaaaaaaaacaaaacaaaaacaccaaacaaa
caggaaaatcaaccccaggctggagagatggctcagcagttaagagcactga
ctgttcttccaaaggtcctgagttcaaatcccagcaagcac
atggtggctcacaaccatctgacatctgatgccctctacaggtgtgtctga
agatagctacagtgtacttacatataaataataaatctttttttaaaa
aaagaagaaaagaaaaatcaacccctattctggcttgatttttgtgatggtt
tcaagatcttgttgttgctgctgttttgttttgttaatttctgagatgat
ggagtcttgtacaacacaggctagctctaactcctgattctacctctacct
ccaaaatgctgagattataataaacaagtttcatgactcccagtac
atatattgtttgtttggttggttggtttggtttgttttttttcgagacaag
gttcctctgtatagccctggctgtcctggaactcactttgtagaccagtctg
gcctcgaactcagaaatctgcctgcctctgcctcccaagtgctgggaatac
ttttttaatttagaaattaatttaagaagcatgttgggtgccgggt
gtggtggcgcactcctttaatcccagcactcgggaggcagaggcaggcgga
tttctgagttcgaggccagactggtctacaaagtgagttcca
ggacagccagggctacacagagaaaccctgtctcaaaaaaccaaaaaaaaa
aaaaaaaaaaaaagcatgttggatctccaaatttgaggcc
agcctggtctacagcacaagttccaggacaaacacagacaaaccctatttca
aacatacatagggtcaggaggcaaaggcaggtagatctct
gtgagtaccagtctcgcttgcctggtctatatagtgagttctaggtattca
ggaaataacctagataatgttagccccaagtgctgtcattatttaag
agagagagaaaataaaggaacctaggggctagagaagtggctcaatgatta
tgaaagagtacaagctgctcttccagaggaccagggttctg
atcccagcacccatgtcaagtcactcacaaatccatatcaccaagagattc
accctcggcgcgccactcttcgcgacagctagatctcatcgcc
taggatcgcccgggttgattcgaggctgctaacaaatcgagtcgagcatcg
agcagtgtggttttcaagaggaagcaaaaagcctctccaccc
aggcctggaatgtttccacccaatgtcgagcagtgtggttttgcaagagga
agcaaaaagcctctccacccaggcctggaatgtttccacccaa
tgtcgagcaaaccccgcccagcgtcttgtcattggcgaattcgaacacgca
gatgcagtcggggcggcgcggtcccaggtccacttcgcata
ttaaggtgacgcgtgtggcctcgaacaccgagcgaccctgcagcgacccgc
ttaacagcgtcaacagcgtgccgcagatcttggtggcgtg
aaactcccgcacctcttcggccagcgccttgtagaagcgcgtgccatgga
tcctgatgatgttgttgattcttctaaatcttttgtgatggaaaactt
ttcttcgtaccacgggactaaacctggttatgtagattccattcaaaaag
gtatacaaaagccaaaatctggtacacaaggaaattatgacgatga
ttggaaagggttttatagtaccgacaataaatacgacgctgcgggatactc
tgtagataatgaaaacccgctctctggaaaagctggaggcgtg
gtcaaagtgacgtatccaggactgacgaaggttctcgcactaaaagtgga
taatgccgaaactattaagaaagagttaggtttaagtctcactga
accgttgatggagcaagtcggaacggaagagtttatcaaaaggttcggtga
tggtgcttcgcgtgtagtgctcagccttcccttcgctgagggg
agttctagcgttgaatatattaataactgggaacaggcgaaagcgttaag
cgtagaacttgagattaattttgaaacccgtggaaaacgtggcca
agatgcgatgtatgagtatatggctcaagcctgtgcaggaaatcgtgtca
ggcgatctattgtgaaggaaccttacttctgtggtgtgacataatt
ggacaaactacctacagagatttaaagctctaaggtaaatataaaatttt
taagtgtataatgtgttaaactactgattctaattgtttgtgtatatagat
tccaacctatggaactgatgaatgggagcagtggtggaatgcagatcctag
agctcgctgatcagcctcgactgtgccttctagttgccagcca
tctgttgtttgcccctcccccgtgccttccttgaccctggaaggtgccac
tcccactgtcattcctaataaaatgaggaaattgcatcgcattgtct
gagtaggtgtcattctattctggggggtggggtggggcaggacagcaagggg
gaggattgggaagacaatagcaggcatgctggggatgc
ggtgggctctatggcttctgaggcggaaagaaccagcccgggcggtggag
ctccaattcgccctatagtgagtcgtattacaattcactggcc
gtcgttttacaacgtcgtgactgggaaaaccctggcgttacccaacttaa
tcgccttgcagcacatccccattcgccagctggcgtaatagcga
agaggcccgcaccgatcgcccttcccaacagttgcgcagcctgaatggc
gaatggaaattgtaagcg

### Example 10

### Nucleotide Sequence of the Top Strand of an Example Vector that was Used to Make the Y220C HUPKI p53 with Arginine Instead of Proline at Position 72 (P72R)

Features and locations are listed below. Amp: 576-1433; 5′arm: 2661-5739; E2: 5259-5341; E3: 5625-5646; hTP53 E4-E9: 5740-8477; hE4: 5740-6018; 72 Arg: 5857-5859; hE5: 6776-6959; hE6: 7041-7153; Y220C: 7139-7141; hE7: 7722-7831; hE8: 8175-8311; hE9: 8404-8477; LoxP: 8889-8922; Neo Cassette: 8960-12,718; LoxP: 12,721-12,754; 3′arm: 12,768-17,516; E10: 13,216-13,322: E11: 13,910-14,433: DTA Cassette: 17.568-19.003.

SEQ ID NO: 12

ttaatattttgttaaaattcgcgttaaatttttgttaaatcagctcattttttaacc
aataggccgaaatcggcaaaatcccttataaatcaaaagaatag
accgagatagggttgagtgttgttccagtttggaacaagagtccactattaaagaac
gtggactccaacgtcaaagggcgaaaaaccgtctatc
agggcgatggcccactacgtgaaccatcaccctaatcaagttttttggggtcgag
gtgccgtaaagcactaaatcggaaccctaaagggagcc
cccgatttagagcttgacggggaaagccggcgaacgtggcgagaaaggaagggaa
gaaagcgaaaggagcgggcgctagggcgctgg
caagtgtagcggtcacgctgcgcgtaaccaccacacccgccgcgcttaatgc
gccgctacagggcgcgtcaggtggcacttttcggggaaa
tgtgcgcggaacccctatttgtttatttttctaaatacattcaaatatgtat
ccgctcatgagacaataaccctgataaatgcttcaataatattgaaaa
aggaagagtatgagtattcaacatttccgtgtcgcccttattcccttattg
cggcattttgccttcctgttatgctcacccagaaacgctggtgaaa
gtaaaagatgctgaagatcagttgggtgcacgagtgggttacatcgaactg
gatctcaacagcggtaagatccttgagagttttcgccccgaag
aacgttttccaatgatgagcacttttaaagttctgctatgtggcgcggtat
tatcccgtattgacgccgggcaagagcaactcggtcgccgcatac
actattctcagaatgacttggttgagtactcaccagtcacagaaaagcatc
ttacggatggcatgacagtaagagaattatgcagtgctgccata
accatgagtgataacactgcggccaacttacttctgacaacgatcggagga
ccgaaggagctaaccgcttttttgcacaacatgggggatcat
gtaactcgccttgatcgttgggaaccggagctgaatgaagccataccaaa
cgacgagcgtgacaccacgatgcctgtagcaatggcaacaac
gttgcgcaaactattaactggcgaactacttactctagcttcccggcaa
caattaatagactggatggaggcggataaagttgcaggaccacttc
tgcgctcggcccttccggctggctggtttattgctgataaatctggag
ccggtgagcgtgggtctcgcggtatcattgcagcactggggccaga
tggtaagccctcccgtatcgtagttatctacacgacggggagtcaggc
aactatggatgaacgaaatagacagatcgctgagataggtgcctc
actgattaagcattggtaactgtcagaccaagtttactcatatatacttt
agattgatttaaaacttcatttttaatttaaaaggatctaggtgaagatcc
tttttgataatctcatgaccaaaatcccttaacgtgagttttcgttccac
tgagcgtcagaccccgtagaaaagatcaaaggatcttcttgagatcct
ttttttctgcgcgtaatctgctgcttgcaaacaaaaaaaccaccgcta
ccagcggtggtttgtttgccggatcaagagctaccaactctttttccga
aggtaactggcttcagcagagcgcagataccaaatactgttcttctagtgt
agccgtagttaggccaccacttcaagaactctgtagcaccgcct
acatacctcgctctgctaatcctgttaccagtggctgctgccagtggcgat
aagtcgtgtcttaccgggttggactcaagacgatagttaccggat
aaggcgcagcggtcgggctgaacggggggttcgtgcacacagcccagcttg
gagcgaacgacctacaccgaactgagatacctacagcgt
gagctatgagaaagcgccacgcttcccgaagggagaaaggcggacaggtat
ccggtaagcggcagggtcggaacaggagagcgcacga
gggagcttccagggggaaacgcctggtatattatagtcctgtcgggtttcg
ccacctctgacttgagcgtcgatttttgtgatgctcgtcagggg
ggcggagcctatggaaaaacgccagcaacgcggcctttttacggttcctgg
ccttttgctggcatttgctcacatgttattcctgcgttatcccct
gattctgtggataaccgtattaccgcctttgagtgagctgataccgctcg
ccgcagccgaacgaccgagcgcagcgagtcagtgagcgagga
agcggaagagcgcccaatacgcaaaccgcctctccccgcgcgttggccgat
tcattaatgcagctggcacgacaggtttcccgactggaaa
gcgggcagtgagcgcaacgcaattaatgtgagttagctcactcattaggca
ccccaggattacactttatgcttccggctcgtatgttgtgtgga
attgtgagcggataacaatttcacacaggaaacagctatgaccatgattac
gccaagctcgaaattaaccctcactaaagggaacaaaagctg
gtacgcggccgcaagtgataccagacacagccttcttattatcttagaacc
aacaatatatcggtccttggcccccacccccatgcccatttttc
caatgctgggtgttgaacccggagccttagacataacacacgaactctgca
gctgagctaccctgccagctccgaaagatttgtatactaagttc
tatgaaacattaaggtcgttttattcagggtaaggtgtggcttctggcttc
cctgggtccagtcattgatggtggtgggtagtactgttcgttccatt
ccgtttggggttttgattgacaagccttgcacctttccaactgttctacct
caagagccaaagataaagggtaaaagattcccttcccttatccctgt
caatagcagcctgcctagcttcctcaggatcaaatgagatgagcccctgag
aagagcaaggcccgctgggcctggaaggccagccctggtt
gtactcaaacctctcgagtctattgcattcccagccaacattttcttacac
atccagcctctgtggatactgtgaccctcctgatctggttcttgtga
aaagtttcatattggcaactgttcttaaggaaacactgaaaacctaattac
tactaacgacctggaagatagagcaggaagacctcctattgtcag
tgtggctatgtctccaagactgagacattgggccgccatcccagcatgtgc
gcgcgcgcgcgcgcacacacacacacacacacacagtattc
actaagcaagagttccttgacagggtgaactggcattaacgggtagtggtt
agcgccacagaagcactgctagctcagaacaaatgaggtgct
catacccacagggctgagctcttacatgagtgtgtactgttgggcgtgggg
tggggctggacccagggcttcacagatgagctccgcccaag
gccaaatctcaaaaaatgaaaaggtcaaaaggtaaccattcagatagatacc
ggagatatgatatttacaaaaaatatgataattaaatgctaca
gttttaattatgaatctgaagcagctctgggtctccctgacccattccaggg
ccagccacctcattactcagatgtaggggctatataatacccag
tcattgattcttcatctagctgattgcaagagaactgtgcctaagagcctgt
gacgcactagattgtttgggtggccatattaataaagagtcatt
cacctgcccaggggcagatgtgaccatcgagacagatgaaatagcccatatg
gaaaggtttctcaaactcttaaataaatgagaacttgattgtt
ctacatacaagtgaatatattaaatgttgagattatagtcagtattaacagt
aattattctgaaatgcttgcattgctgttagaaaggaaacatgatgt
cccagggggttggccgaactcaatccccagaacccacttcttataagttgtc
atctgacctccatacgcatgtcttggcacattaaataaggcac
aataaataagtaaatgtctaagtaaggaagataatagctccgtggttagtaa
caagagaaaatgagtgagagagtatcatacggcttaacctctg
aaggaggtggtggtggtgtggctggctggagattggctggctgtgactgtct
ccgaggagctatggcatacaagataaggaagagcctacct
catagcctagggagaacagaaactgtgactttgctcttgtagaggtaaccac
actgattcagccaggaggaagtaagtgctccctagagccctt
ggggaagaaggcagtaggaaacctgctgaatcttcaggaatttgtaaggcgc
tggggacctgtccctagggggcagatgagacactgatgg
gcgtacttagagatttgccatgaagtgggtttgaagaatggagctgtgtgtg
aaatggtggatggggggggggagtccctccccagagggaa
gggaagagagatgagatgtagggtgcagatgtaggggggcttggggctagaa
gtacctccctgattacctgttccttgaagcagtgtgtggtt
cgagaagctgataggaagccaggccaaggcttgagcagcctgaataaaagacg
gaagagctgccccattcctgcttctctggaaatggtgtc
cctcacggccatcttgggtcctgacttcttctcaaaggagcctggccgactt
cttggatacttgtaactttgcgattcccaccctcgcataagtttc
ctgaaataatgactctgaaactcaaaatatatttacaaacacctcggctgtag
attggtttgttctctgacttgcttataacttaatatccctcttattct
aacctaagttctgccacgtggttggttacctctgctcagcccccggcttctg
tcctccatgttcctgggggaatccctacactttcagaatttaatttc
cctactggatgtcccaccttctttttattctaccctttcctataagccatag
gggtttgtttgtttgtatgttttttaattgacaagttatgcatccatacagt
acacaatctcttctctctacagatgactgccatggaggagtcacagtcggata
tcagcctcgagctccctctgagccaggagacattttcaggct
tatggaaactgtgagtggatattttggggcccttaagatacatcccgccatac
ctgtatcctccccttgcctgagagaaacaaaaacagtagtgt
tcaaacatggtatggtgttgggtgtctgtaaatcctgcggggcggggtggcg
gggggggggggggactgcagggtctcagaagtttgaggt
catcattgactacatagcaagttggaggccagcctgggataagtgagattct
gtcttcaaaaaatggaaggaaatcaggaactaactctctgctc
ttgttttccagacttcctccagaagatatcctggtaaggcccagagcagaaag
ggacttgggattggtgttgggctggtaggctgagaacaca
gtcctgagggttcttattgtcccatccacagtcccccttgccgtcccaagcaa
tggatgatttgatgctgtccccggacgatattgaacaatggtt
cactgaagacccaggtccagatgaagctcccagaatgccagaggctgctcccc
gcgtggcccctgcaccagcagctcctacaccggcggc
ccctgcaccagccccctcctggcccctgtcatcttctgtcccttcccagaaaa
cctaccagggcagctacggtttccgtctgggcttcttgcattc
tgggacagccaagtctgtgacttgcacggtcagttgccctgaggggctggcttc
catgagacttcaatgcctggccgtatccccctgcatttcttt
tgtttggaactttgggattcctcttcaccattggcttcctgtcagtgtttttt
tatagtttacccacttaatgtgtgatctctgactcctgtcccaaagttg
aatattccccccttgaatttgggatttatccatcccatcacaccctcagcatct
ctcctggggatgcagaacttttctttttcttcatccacgtgtattc
cttggatttgaaaataagctcctgaccaggcttggtggctcacacctgcaatcc
cagcactctcaaagaggccaaggcaggcagatcacctg
agcccaggagttcaagaccagcctgggtaacatgatgaaacctcgtctctacaa
aaaaatacaaaaaattagccaggcatggtggtgcacac
ctatagtcccagccacttaggaggctgaggtgggaagatcacttgaggccagga
gatggaggctgcagtgagctgtgatcacaccactgtgc
tccagcctgagtgacagagcaagaccctatctcaaaaaaaaaaaaaaaaaagaa
aagctcctgaggtgtagacgccaactctctctagctcg
ctagtgggttgcaggaggtgcttacgcatgtttgtttattgctgccgtcttcca
gttgctttatctgttcacttgtgccctgactttcaactctgtctcct
tcctcttcctacagtactcccctgccctcaacaagatgttttgccaactggcca
agacctgccctgtgcagctgtgggttgattccacacccccgc
ccggcacccgcgtccgcgccatggccatctacaagcagtcacagcacatgacgg
aggttgtgaggcgctgcccccaccatgagcgctgctc
agatagcgatggtgagcagctggggctggagagacgacagggctggttgcccag
ggtccccaggcctctgattcctcactgattgctcttag
gtctggcccctcctcagcatcttatccgagtggaaggaaatttgcgtgtggagt
atttggatgacagaaacacttttcgacatagtgtggtggtgc
cctgtgagccgcctgaggtctggtttgcaactggggtctctgggaggaggggtt
aagggtggttgtcagtggccctccaggtgagcagtagg
ggggctttctcctgctgcttatttgacctccctataaccccatgagatgtgcaa
agtaaatgggtttaactattgcacagttgaaaaaactgaagctt
acagaggctaagggcctcccctgcttggctgggcgcagtggctcatgcctgtaa
tcccagcactttgggaggccaaggcaggcggatcacg
aggttgggagatcgagaccatcctggctaacggtgaaaccccgtctctactgaa
aaatacaaaaaaaaattagccgggcgtggtgctgggca
cctgtagtcccagctactcgggaggctgaggaaggagaatggcgtgaacctggg
cggtggagcttgcagtgagctgagatcacgccactgc
actccagcctgggcgacagagcgagattccatctcaaaaaaaaaaaaaaaaggcc
tcccctgcttgccacaggtctccccaaggcgcactg
gcctcatcttgggcctgtgttatctcctaggttggctctgactgtaccaccatc
cactacaactacatgtgtaacagttcctgcatgggcggcatga
accggaggcccatcctcaccatcatcacactggaagactccaggtcaggagcca
cttgccaccctgcacactggcctgctgtgccccagcct
ctgcttgcctctgacccctgggcccacctcttaccgatttcttccatactactac
ccatccacctctcatcacatccccggcggggaatctccttac
tgctcccactcagttttcttttctctggattgggacctcttaacctgtggcttc
tcctccacctacctggagctggagcttaggctccagaaaggac
aagggtggttgggagtagatggagcctggttttttaaatgggacaggtaggacc
tgatttccttactgcctcttgcttctcttttcctatcctgagtag
tggtaatctactgggacggaacagattgaggtgcgtgtttgtgcctgtcctggg
agagaccggcgcacagaggaagagaatctccgcaaga
aaggggagcctcaccacgagctgcccccagggagcactaagcgaggtaagcaagc
aggacaagaagcggtggaggagaccaagggtg
cagttatgcctcagattcacttttatcacattccttgcctattcctagcactgc
ccaacaacaccagctcctctccccagccaaagaagaaacca
ctggatggagaatatttcacccttcaggtaccaaggctggagagtcgcatgccag
agacaagctgtacccattattgcctctgtctctcgcatgt
ataaaatagtgttattagcaggttgccaggtctttttcagtggattatctctagc
cgtgacattagcttgagagctcatgctctgaggctgtgcctcc
tccgacagtggttctcagtgtaactaacttgacaacaccaacttacaccataaga
caggtgctcctccactggggatgggaacatgtcctagga
aactcaccataaattgaaaataacacacgacaaaaatgtgtttagaggcaggcct
ggtggcacctgcctgtaattacagcactggtcgacctgc
agccaagctatcgaattcctgcagcccaattccgatcatattcaataaccctta
atataacttcgtataatgtatgctatacgaagttattaggtctga
agaggagtttacgtccagccaagctagctccatgggccaggcaaatatccctta
ccagcctcacagagacctcccccaccccccgcaaccct
agagttatttactagtgagggacaagtggacaatggtgctgttgtgggcccca
ccctgtgtcccctgtgcccacagtggtcactctgcttggca
ggcaggtgttgcaggctggctgctccaggccctggcaggaggtactgaaggacc
tggtaggctcagatgccctggatgccaaggcactgct
ggagtacttccaaccggtcagccagtggctggaagagcagaatcagcggaatg
gcgaagtcctaggctggccagagaatcagtggcgtcc
accgttacccgacaactatccagagggcattggtaaagctctgagtgagggtg
gactgggaccaagagaagtcctggcctctggcctctggc
ttctgggtcaaagcctcagcatcctggtcactttgctgccagctgagccccagt
gtcattgcttcagtgccaagccacccctgggctcatcctca
gggccctaagcagaaatgggtatgtattctctcagggtcctagagacagtgtg
cccaagcctgagggcccttggggtcaggctggctggca
cattgctctatgaggtcacactgcaggcttggctcttattggccggtgatggga
gcttcagggctctgattcctgcggccatgcccaagaagaa
gaggaaggtgtccaatttactgaccgtacaccaaaatttgcctgcattaccgg
tcgatgcaacgagtgatgaggttcgcaagaacctgatggac
atgttcagggatcgccaggcgttttctgagcatacctggaaaatgcttctgtc
cgtttgccggtcgtgggcggcatggtgcaagttgaataaccg
gaaatggtttcccgcagaacctgaagatgttcgcgattatcttctatatcttca
ggcgcgcggtctggcagtaaaaactatccagcaacatttggg
ccagctaaacatgcttcatcgtcggtccgggctgccacgaccaagtgacagcaa
tgctgtttcactggttatgcggcggatccgaaaagaaaa
cgttgatgccggtgaacgtgcaaaacaggctctagcgttcgaacgcactgatt
tcgaccaggttcgttcactcatggaaaatagcgatcgctgc
caggatatacgtaatctggcatttctggggattgcttataacaccctgttacgt
atagccgaaattgccaggatcagggttaaagatatctcacgta
ctgacggtgggagaatgttaatccatattggcagaacgaaaacgctggttagca
ccgcaggtgtagagaaggcacttagcctgggggtaact
aaactggtcgagcgatggatttccgtctctggtgtagctgatgatccgaata
actacctgttttgccgggtcagaaaaaatggtgttgccgcgcc
atctgccaccagccagctatcaactcgcgccctggaagggatttttgaagcaa
ctcatcgattgatttacggcgctaaggtaaatataaaattttta
agtgtataatgtgttaaactactgattctaattgtttgtgtattttaggatgac
tctggtcagagatacctggcctggtctggacacagtgcccgtgtc
ggagccgcgcgagatatggcccgcgctggagtttcaataccggagatcatgcaa
gctggtggctggaccaatgtaaatattgtcatgaactat
atccgtaacctggatagtgaaacaggggcaatggtgcgcctgctggaagatggc
gattagccattaacgcgtaaatgattgctataattatttga
tatttatggtgacatatgagaaaggatttcaacatcgacggaaaatatgtagtg
ctgtctgtaagcactaatattcagtcgccagccgtcattgtca
ctgtaaagctgagcggcaataaaaagacagaataaaacgcacgggtgttgggt
cgtttgttcggtcgagctcgcgaagctagcttggctgcag
gtcgtcgaaattctaccgggtaggggaggcgcttttcccaaggcagtctggag
catgcgctttagcagccccgctgggcacttggcgctacac
aagtggcctctggcctcgcacacattccacatccaccggtaggcgccaaccgg
ctccgttattggtggccccttcgcgccaccttctactcctc
ccctagtcaggaagttcccccccgccccgcagctcgcgtcgtgcaggacgtg
acaaatggaagtagcacgtctcactagtctcgtgcagatg
gacagcaccgctgagcaatggaagcgggtaggcctttggggcagcggccaata
gcagattgctccttcgattctgggctcagaggctggg
aaggggtgggtccgggggcgggctcaggggcgggctcaggggcggggcgggcg
cccgaaggtcctccggaggcccggcattctgcac
gcttcaaaagcgcacgtctgccgcgctgttctcctcttcctcatctccgggcc
tttcgacctgcagcctgttgacaattaatcatcggcatagtata
tcggcatagtataatacgacaaggtgaggaactaaaccatgggatcggccatt
gaacaagatggattgcacgcaggttctccggccgcttggg
tggagaggctattcggctatgactgggcacaacagacaatcggctgctctgatg
ccgccgtgttccggctgtcagcgcaggggcgcccggtt
ctttttgtcaagaccgacctgtccggtgccctgaatgaactgcaggacgagg
cagcgcggctatcgtggctggccacgacgggcgttccttgc
gcagctgtgctcgacgttgtcactgaagcgggaagggactggctgctattggg
cgaagtgccggggcaggatctcctgtcatctcaccttgct
cctgccgagaaagtatccatcatggctgatgcaatgcggcggctgcatacgc
ttgatccggctacctgcccattcgaccaccaagcgaaacat
cgcatcgagcgagcacgtactcggatggaagccggtcttgtcgatcaggatga
tctggacgaagagcatcaggggctcgcgccagccgaa
ctgttcgccaggctcaaggcgcgcatgcccgacggcgatgatctcgtcgtga
cccatggcgatgcctgcttgccgaatatcatggtggaaaat
ggccgcttttctggattcatcgactgtggccggctgggtgtggcggaccgcta
tcaggacatagcgttggctacccgtgatattgctgaagagc
ttggcggcgaatgggctgaccgcttcctcgtgctttacggtatcgccgctccc
gattcgcagcgcatcgccttctatcgccttcttgacgagttctt
ctgaggggatcaattctctagagctcgctgatcagcctcgactgtgccttcta
gttgccagccatctgttgtttgcccctcccccgtgccttccttg
accctggaaggtgccactcccactgtcattcctaataaaatgaggaaattgca
tcgcattgtctgagtaggtgtcattctattctggggggtggg
gtggggcaggacagcaagggggaggattgggaagacaatagcaggcatgctgg
ggatgcggtgggctctatggcttctgaggcggaaag
aaccagctggggctcgactagagcttgcggaacccttaatataacttcgtata
atgtatgctatacgaagttattaggtccctcgagaagagggt
ggttcaaagctggcaagaaaaagtaacccaaggaaaggacattggggtttaag
ggtataactcagttccaggatacttgccaagtgtgtgcca
ggccccggattaggtccccagcggctcacggtgcattagtccacctaacccac
aactggactctacagctcagctcccggttgccctgttaag
cgtctgttccctctaactgaaaagatccaagcttgttgtacacgttctactga
atgcctgttactttcacaccatcttattaaagatgatctccccccc
ccccaaaaaaaaaaacaaaacaaaacaaaacaaaaacctgtaagtggagccag
cttaagttgggaaccaactttcagaaagaaagttgttaa
aatcgtgaaagtggttgtgtgaccttgtccagtgcttccatctcacttcatct
ctgctgcagatccgcgggcgtaaacgcttcgagatgttccggg
agctgaatgaggccttagagttaaaggatgcccatgctacagaggagtctgga
gacagcagggctcactccaggtaagtggcctggggcag
cgcctgcctgtggtgctctacccagacctccctccagctcagcattgtagtga
aagataaaaaccccaccctgctagatgcttagggctgcac
cctacgagaactgacttcttgactttttaggctctgttaaggggtatgaggga
caaggtatggtgtcatgctcctataatctcagcagtaaggaag
acaaagtcaggaggatttggggaagtttgaagccttcataaactatataaaac
tttaggccagctagggctagctacaatagcaagaccctgtct
catggtgatggtgatgatggtggtggtggtgacagttgtgataataatggcag
tggtggtgatgatgatggtggtggtgatgatggtgatggtga
aagggaggataaactgattctcagaagtattccagtgtgttctgtgaatatcc
ctacccatagtagaagccatcttaaattcctttttttcagcctcca
gcctagagccttccaagccttgatcaaggaggaaagcccaaactgctagctccc
atcacttcatccctccccttttctgtcttcctatagctacctg
aagaccaagaagggccagtctacttcccgccataaaaaaacaatggtcaagaaa
gtggggcctgactcagactgactgcctctgcatcccgt
ccccatcaccagcctccccctctccttgctgtcttatgacttcagggctgaga
cacaatcctcccggtcccttctgctgccttttttaccttgtagcta
gggctcagccccctctctgagtagtggttcctggcccaagttggggaataggtt
gatagttgtcaggtctctgctggcccagcgaaattctatcc
agccagttgttggaccctggcacctacaatgaaatctcaccctaccccacacc
ctgtaagattctatcttgggccctcatagggtccatatcctcc
agggcctactttccttccattctgcaaagcctgtctgcatttatccacccccc
accctgtctccctcttttttatttttacccattttatatatcaatttcc
tattttacaataaaattttgttatcacttatatggttttgagaggttgatatc
agcataagctgtctgggcccccaggggcaggatgaatttgggagg
tacccacctgagtccaggcagtctgttggagtcaggggtggggaacttgggtt
ccagaagaggaacaaagccggggactgggtcagtctgt
gggctgcatgacaacaagagagcagggtgactccattcataacttgggaacca
actgtccctcctccctcctgccaggactggcacatggtcc
ttacccacccctacttctagggttgggctcctgctgtcctctggggagcctct
caccaaggattaagggatttaaatgtctgatatagcaaacctg
agcctctggagtgaccatctgctccacaagaaaggatcagggtgcctgggttcc
acagggaagggggtggctgttcctggatgaagagaca
agtgggaggcccgccagctggggtcccaggaaactgggagcagttaaggtgag
gctaggggccttcccagcatccccaaactccgggcct
cacgccaggcaagtgaatgaatcgaagattgcactttgccaggaaatcattgaa
agggcttcttggagtagggaggagagtcagagttggg
gagcctaacaccctctcacccacccccttcctcagtgctgctggctcccagaga
gcgggactaggccaagctctccccatagcctgctgagc
cagtgccagtagggagagcagtcggtgagcacatggcttggccatggctcttgg
taacagagagaggggtactgaggtccagaggcacttt
ggggccactgtcctctgcctgtcccaggcttaaagagccagtgagagtcattgc
cttgcccgagggtcctatacaagttgggagggagcagc
cccttagcctcaaaattttgtacttgtgagtaccaaatattttcgcagcagact
agggtctatcttataactgttgtgggaatgtgtgatgccagtca
aacccactacgcttacccctgtttttgtcccacagattactgagaactaaatcca
aagaactgtgtttaagggccacaaatttgagcccttatctc
aaaaagggaggaggggctaaaacaattgccaaatgaaaagctatctctgcatt
tgtgcctgtatggtaaacgttaagactgacagagaactact
caacaaaagctcctccacccaactcattattatttcagacagtaccttgctaac
taagtagcccaggctaccctcaacattggtctattatttagct
ttccaagtggagggagggatcgatgggctaccatacttccttaccttccccatt
tccctatcttttgtccttaattctgtccttgtacctcaagtcttca
gaaagcaggtccattttggtacctcaaatcccttctctgagagctgatatcaatg
gacaaaggataatgaggttatgcttctcctgcaagctcttct
atgtaacttcacatcagtcacttacctcccaggttccacttccgggctgtttcca
ttgatgcatcaactaggcatcgatccataaagtacttgacatt
cacagggaccctatgtctgctatgatgtctgcccaagaggtaaatacctcaggc
cttactctattgaggtctaaaatcaaacctcccaatctctaat
cttgtaactctaacctacttttacattttaggtcaatatattttttatttatgt
attttgagaaatggtggcgtgccagaccatgataagcaagacattgta
ccagcaattaaccccccacccccaactccatagctgggtggtgatggagcacac
ctttaatcctggcacttgggacacagaggcaggcagag
ttcagcctagtaaacagagtgagttccagtccagccagggctatacagagaaac
cctgtctcaaaaaaaacaaaacaaaaacaccaaacaaa
caggaaaatcaaccccaggctggagagatggctcagcagttaagagcactgact
gttcttccaaaggtcctgagttcaaatcccagcaagcac
atggtggctcacaaccatctgacatctgatgccctctacaggtgtgtctgaaga
tagctacagtgtacttacatataaataataaatattttttaaaa
aaagaagaaaagaaaaatcaacccctattctggcttgatttttgtgatggtttc
aagatcttgttgttgctgctgttttgttttgttaatttctgagatgat
ggagtcttgtacaacacaggctagctctaactcctgattctacctctacctccaa
aatgctgagattataataaacaagtttcatgactcccagtac
atatattgtttgtttggttggttggtttggtttgttttttttcgagacaaggtt
cctctgtatagccctggctgtcctggaactcactttgtagaccagtctg
gcctcgaactcagaaatctgcctgcctctgcctcccaagtgctgggaatactttt
ttaatttagaaattaatttaagaagcatgttgggtgccgggt
gtggtggcgcactcctttaatcccagcactcgggaggcagaggcaggcggatttc
tgagttcgaggccagactggtctacaaagtgagttcca
ggacagccagggctacacagagaaaccctgtctcaaaaaaccaaaaaaaaaaaaa
aaaaaaaaagcatgttggatctccaaatttgaggcc
agcctggtctacagcacaagttccaggacaaacacagacaaaccctatttcaaac
atacatagggtcaggaggcaaaggcaggtagatctct
gtgagtaccagtctcgcttgcctggtctatatagtgagttctaggtattcaggaa
ataacctagataatgttagccccaagtgctgtcattatttaag
agagagagaaaataaaggaacctaggggctagagaagtggctcaatgattatgaa
agagtacaagctgctcttccagaggaccagggttctg
atcccagcacccatgtcaagtcactcacaaatccatatcaccaagagattcacc
ctcggcgcgccactcttcgcgacagctagatctcatcgcc
taggatcgcccgggttgattcgaggctgctaacaaatcgagtcgagcatcgagca
gtgtggttttcaagaggaagcaaaaagcctctccaccc
aggcctggaatgtttccacccaatgtcgagcagtgtggttttgcaagaggaagca
aaaagcctctccacccaggcctggaatgtttccacccaa
tgtcgagcaaaccccgcccagcgtcttgtcattggcgaattcgaacacgcagatg
cagtcggggcggcgcggtcccaggtccacttcgcata
ttaaggtgacgcgtgtggcctcgaacaccgagcgaccctgcagcgacccgcttaa
cagcgtcaacagcgtgccgcagatcttggtggcgtg
aaactcccgcacctcttcggccagcgccttgtagaagcgcgtgccatggatcctg
atgatgttgttgattcttctaaatcttttgtgatggaaaactt
ttcttcgtaccacgggactaaacctggttatgtagattccattcaaaaaggtata
caaaagccaaaatctggtacacaaggaaattatgacgatga
ttggaaagggttttatagtaccgacaataaatacgacgctgcgggatactctgta
gataatgaaaacccgctctctggaaaagctggaggcgtg
gtcaaagtgacgtatccaggactgacgaaggttctcgcactaaaagtggataatg
ccgaaactattaagaaagagttaggtttaagtctcactga
accgttgatggagcaagtcggaacggaagagtttatcaaaaggttcggtgatggt
gcttcgcgtgtagtgctcagccttcccttcgctgagggg
agttctagcgttgaatatattaataactgggaacaggcgaaagcgttaagcgtag
aacttgagattaattttgaaacccgtggaaaacgtggcca
agatgcgatgtatgagtatatggctcaagcctgtgcaggaaatcgtgtcaggcga
tctattgtgaaggaaccttacttctgtggtgtgacataatt
ggacaaactacctacagagatttaaagctctaaggtaaatataaaatttttaagt
gtataatgtgttaaactactgattctaattgtttgtgtatatagat
tccaacctatggaactgatgaatgggagcagtggtggaatgcagatcctagagctc
gctgatcagcctcgactgtgccttctagttgccagcca
tctgttgtttgcccctcccccgtgccttccttgaccctggaaggtgccactccca
ctgtcattcctaataaaatgaggaaattgcatcgcattgtct
gagtaggtgtcattctattctggggggtggggtggggcaggacagcaagggggag
gattgggaagacaatagcaggcatgctggggatgc
ggtgggctctatggcttctgaggcggaaagaaccagcccgggcggtggagctcca
attcgccctatagtgagtcgtattacaattcactggcc
gtcgttttacaacgtcgtgactgggaaaaccctggcgttacccaacttaatcgcc
ttgcagcacatccccctttcgccagctggcgtaatagcga
agaggcccgcaccgatcgcccttcccaacagttgcgcagcctgaatggcgaatg
gaaattgtaagcg

### Example 11

### Amino Acid Sequence of One of the Y220C HUPKI p53 Isoform Translated from the Example Vector, Which Includes Proline Instead of Arginine at Position 72 (P72)

The corresponding amino acid positions in mouse exon 3 were shifted 3 amino acids higher relative to human p53, but the corresponding amino acid positions in human p53 in exons 4 to 9 were shifted 3 amino acids higher relative to mouse p53.

SEQ ID NO: 13

MTAMEESQSDISLELPLSQETFSGLWKLLPPEDILSPLPSQAMDDLMLS
PDDIEQWFTEDPGPDEAPRMPEAAPPVAPAPAAPTPAAPAPAPSWPLSS
SVPSQKTYQGSYGFRLGFLHSGTAKSVTCTYSPALNKMFCQLAKTCPVQ
LWVDSTPPPGTRVRAMAIYKQSQHMTEVVRRCPHHERCSDSDGLAPPQH
LIRVEGNLRVEYLDDRNTFRHSVVVPCEPPEVGSDCTTIHYNYMCNSSC
MGGMNRRPILTIITLEDSSGNLLGRNSFEVRVCACPGRDRRTEEENLRK
KGEPHHELPPGSTKRALPNNTSSSPQPKKKPLDGEYFTLQIRGRKRFEM
FRELNEALELKDAHATEESGDSRAHSSYLKTKKGQSTSREIKKTMVKKV
GPDSD

### Example 12

### Amino Acid Sequence of One of the Y220C HUPKI p53 Isoform Translated from the Example Vector, Which Includes Arginine Instead of Proline at Position 72 (P72R)

The corresponding amino acid positions in mouse exon 3 were shifted 3 amino acids higher relative to human p53, but the corresponding amino acid positions in human p53 in exons 4 to 9 were shifted 3 amino acids higher relative to mouse p53.

SEQ ID NO: 14

MTAMEESQSDISLELPLSQETFSGLWKLLPPEDILSPLPSQAMDDLMLS
PDDIEQWFTEDPGPDEAPRMPEAAPRVAPAPAAPTPAAPAPAPSWPLSS
SVPSQKTYQGSYGFRLGFLHSGTAKSVTCTYSPALNKMFCQLAKTCPVQ
LWVDSTPPPGTRVRAMAIYKQSQHMTEVVRRCPHHERCSDSDGLAPPQH
LIRVEGNLRVEYLDDRNTFRHSVVVPCEPPEVGSDCTTIHYNYMCNSSC
MGGMNRRPILTIITLEDSSGNLLGRNSFEVRVCACPGRDRRTEEENLRK
KGEPHHELPPGSTKRALPNNTSSSPQPKKKPLDGEYFTLQIRGRKRFEM
FRELNEALELKDAHATEESGDSRAHSSYLKTKKGQSTSREIKKTMVKKV
GPDSD

### Example 13

### Example of Endogenous Mouse Trp53 (p53) Nucleotide and Amino Acid Sequences, Broken Out by Exon

| Description | Sequence listing |
|---|---|
| Exon 1 nucleotide sequence | TTTCCCCTCCCACGTGCTCACCCTGGCTAAAGTTCTGTAG |
| (SEQ ID NO: 15) | CTTCAGTTCATTGGGACCATCCTGGCTGTAGGTAGCGACT |
| | ACAGTTAGGGGGCACCTAGCATTCAGGCCCTCATCCTCCT |
| | CCTTCCCAGCAGGGTGTCACGCTTCTCCGAAGACTGG |
| Exon 1 amino acid sequence | Non-coding |
| Exon 2 nucleotide sequence | ATGACTGCCATGGAGGAGTCACAGTCGGATATCAGCCTC |
| (SEQ ID NO: 16) | GAGCTCCCTCTGAGCCAGGAGACATTTTCAGGCTTATGGA |
| | AACT |
| Exon 2 amino acid sequence^{a} | MTAMEESQSDISLELPLSQETFSGLWK |
| (SEQ ID NO: 17) | |
| Exon 3 nucleotide sequence | ACTTCCTCCAGAAGATATCCTG |
| (SEQ ID NO: 18) | |
| Exon 3 amino acid sequence^{b} | LLPPEDIL |
| (SEQ ID NO: 19) | |
| Exon 4 nucleotide sequence | CCATCACCTCACTGCATGGACGATCTGTTGCTGCCCCAGG |
| (SEQ ID NO: 20) | ATGTTGAGGAGTTTTTTGAAGGCCCAAGTGAAGCCCTCCG |
| | AGTGTCAGGAGCTCCTGCAGCACAGGACCCTGTCACCGA |
| | GACCCCTGGGCCAGTGGCCCCTGCCCCAGCCACTCCATGG |
| | CCCCTGTCATCTTTTGTCCCTTCTCAAAAAACTTACCAGG |
| | GCAACTATGGCTTCCACCTGGGCTTCCTGCAGTCTGGGAC |
| | AGCCAAGTCTGTTATGTGCACG |
| Exon 4 amino acid sequence | PSPHCMDDLLLPQDVEEFFEGPSEALRVSGAPAAQDPVTETP |
| (SEQ ID NO: 21) | GPVAPAPATPWPLSSFVPSQKTYQGNYGFHLGFLQSGTAKS |
| | VMCT |
| Exon 5 nucleotide sequence | TACTCTCCTCCCCTCAATAAGCTATTCTGCCAGCTGGCGA |
| (SEQ ID NO: 22) | AGACGTGCCCTGTGCAGTTGTGGGTCAGCGCCACACCTCC |
| | AGCTGGGAGCCGTGTCCGCGCCATGGCCATCTACAAGAA |
| | GTCACAGCACATGACGGAGGTCGTGAGACGCTGCCCCCA |
| | CCATGAGCGCTGCTCCGATGGTGATG |
| Exon 5 amino acid sequence^{c} | YSPPLNKLFCQLAKTCPVQLWVSATPPAGSRVRAMAIYKKS |
| (SEQ ID NO: 23) | QHMTEVVRRCPHHERCSDGD |
| Exon 6 nucleotide sequence | GCCTGGCTCCTCCCCAGCATCTTATCCGGGTGGAAGGAAA |
| (SEQ ID NO: 24) | TTTGTATCCCGAGTATCTGGAAGACAGGCAGACTTTTCGC |
| | CACAGCGTGGTGGTACCTTATGAGCCACCCGAG |
| Exon 6 amino acid sequence^{d} | GLAPPQHLIRVEGNLYPEYLEDRQTFRHSVVVPYEPPE |
| (SEQ ID NO: 25) | |
| Exon 7 nucleotide sequence | GCCGGCTCTGAGTATACCACCATCCACTACAAGTACATGT |
| (SEQ ID NO: 26) | GTAATAGCTCCTGCATGGGGGGCATGAACCGCCGACCTA |
| | TCCTTACCATCATCACACTGGAAGACTCCAG |
| Exon 7 amino acid sequence^{e} | AGSEYTTIHYKYMCNSSCMGGMNRRPILTIITLEDS |
| (SEQ ID NO: 27) | |
| Exon 8 nucleotide sequence | TGGGAACCTTCTGGGACGGGACAGCTTTGAGGTTCGTGTT |
| (SEQ ID NO: 28) | TGTGCCTGCCCTGGGAGAGACCGCCGTACAGAAGAAGAA |
| | AATTTCCGCAAAAAGGAAGTCCTTTGCCCTGAACTGCCCC |
| | CAGGGAGCGCAAAGAGAG |
| Exon 8 amino acid sequence^{f} | SGNLLGRDSFEVRVCACPGRDRRTEEENFRKKEVLCPELPPG |
| (SEQ ID NO: 29) | SAKR |
| Exon 9 nucleotide sequence | CGCTGCCCACCTGCACAAGCGCCTCTCCCCCGCAAAAGA |
| (SEQ ID NO: 30) | AAAAACCACTTGATGGAGAGTATTTCACCCTCAAG |
| Exon 9 amino acid sequence^{g} | ALPTCTSASPPQKKKPLDGEYFTLK |
| (SEQ ID NO: 31) | |
| Exon 10 nucleotide sequence | ATCCGCGGGCGTAAACGCTTCGAGATGTTCCGGGAGCTG |
| (SEQ ID NO: 32) | AATGAGGCCTTAGAGTTAAAGGATGCCCATGCTACAGAG |
| | GAGTCTGGAGACAGCAGGGCTCACTCCAG |
| Exon 10 amino acid sequence^{h} | IRGRKRFEMFRELNEALELKDAHATEESGDSRAHS |
| (SEQ ID NO: 33) | |
| Exon 11 nucleotide sequence | CTACCTGAAGACCAAGAAGGGCCAGTCTACTTCCCGCCA |
| (SEQ ID NO: 34) | TAAAAAAACAATGGTCAAGAAAGTGGGGCCTGACTCAGA |
| | CTGACTGCCTCTGCATCCCGTCCCCATCACCAGCCTCCCC |
| | CTCTCCTTGCTGTCTTATGACTTCAGGGCTGAGACACAAT |
| | CCTCCCGGTCCCTTCTGCTGCCTTTTTTACCTTGTAGCTAG |
| | GGCTCAGCCCCCTCTCTGAGTAGTGGTTCCTGGCCCAAGT |
| | TGGGGAATAGGTTGATAGTTGTCAGGTCTCTGCTGGCCCA |
| | GCGAAATTCTATCCAGCCAGTTGTTGGACCCTGGCACCTA |
| | CAATGAAATCTCACCCTACCCCACACCCTGTAAGATTCTA |
| | TCTTGGGCCCTCATAGGGTCCATATCCTCCAGGGCCTACT |
| | TTCCTTCCATTCTGCAAAGCCTGTCTGCATTTATCCACCCC |
| | CCACCCTGTCTCCCTCTTTTTTTTTTTTTTACCCCTTTTTAT |
| | ATATCAATTTCCTATTTTACAATAAAATTTTGTTATCACTT |
| | A |
| Exon 11 amino acid sequenceⁱ | SYLKTKKGQSTSREIKKTMVKKVGPDSD |
| (SEQ ID NO: 35) | |

^{a}The last 2 nucleotides of the exon are for the first amino acid in Exon 3
^{b}The first amino acid of the exon utilizes the last 2 nucleotides of exon 2
^{c}The last nucleotide of the exon is for the first amino acid in Exon 6
^{d}The first amino acid of the exon utilizes the last nucleotide of Exon 5
^{e}The last 2 nucleotides of the exon are for the first amino acid in Exon 8
^{f}The first amino acid of the exon utilizes the last 2 nucleotides of Exon 7, and the last nucleotide of the exon is for the first amino acid in Exon 9
^{g}The first amino acid of the exon utilizes the last nucleotide of Exon 8
^{h}The last 2 nucleotides of the exon are for the first amino acid in Exon 11
ⁱThe first amino acid of the exon utilizes the last 2 nucleotides of Exon 10

### Example 14

### An example of Endogenous Human TP53 (p53) Nucleotide and Amino Acid Sequences, Broken Out by Exon

| Description | Sequence Listing |
|---|---|
| Exon 1 nucleotide sequence | CTCAAAAGTCTAGAGCCACCGTCCAGGGAGCAGGTAGCT |
| (SEQ ID NO: 36) | GCTGGGCTCCGGGGACACTTTGCGTTCGGGCTGGGAGCG |
| | TGCTTTCCACGACGGTGACACGCTTCCCTGGATTGG |
| Exon 1 amino acid sequence | Non-coding |
| Exon 2 nucleotide sequence | CAGCCAGACTGCCTTCCGGGTCACTGCCATGGAGGAGCC |
| (SEQ ID NO: 37) | GCAGTCAGATCCTAGCGTCGAGCCCCCTCTGAGTCAGGA |
| | AACATTTTCAGACCTATGGAAACT |
| Exon 2 amino acid sequence^{a} | MEEPQSDPSVEPPLSQETFSDLWK |
| (SEQ ID NO: 38) | |
| Exon 3 nucleotide sequence | ACTTCCTGAAAACAACGTTCTG |
| (SEQ ID NO: 39) | |
| Exon 3 amino acid sequence^{b} | LLPENNVL |
| (SEQ ID NO: 40) | |
| Exon 4 nucleotide sequence | TCCCCCTTGCCGTCCCAAGCAATGGATGATTTGATGCTGT |
| (SEQ ID NO: 41) | CCCCGGACGATATTGAACAATGGTTCACTGAAGACCCAG |
| | GTCCAGATGAAGCTCCCAGAATGCCAGAGGCTGCTCCCC |
| | CCGTGGCCCCTGCACCAGCAGCTCCTACACCGGCGGCCCC |
| | TGCACCAGCCCCCTCCTGGCCCCTGTCATCTTCTGTCCCTT |
| | CCCAGAAAACCTACCAGGGCAGCTACGGTTTCCGTCTGG |
| | GCTTCTTGCATTCTGGGACAGCCAAGTCTGTGACTTGCAC |
| | G |
| Exon 4 amino acid sequence | SPLPSQAMDDLMLSPDDIEQWFTEDPGPDEAPRMPEAAPPV |
| (SEQ ID NO: 42) | APAPAAPTPAAPAPAPSWPLSSSVPSQKTYQGSYGFRLGFLH |
| | SGTAKSVTCT |
| Exon 5 nucleotide sequence | TACTCCCCTGCCCTCAACAAGATGTTTTGCCAACTGGCCA |
| (SEQ ID NO: 43) | AGACCTGCCCTGTGCAGCTGTGGGTTGATTCCACACCCCC |
| | GCCCGGCACCCGCGTCCGCGCCATGGCCATCTACAAGCA |
| | GTCACAGCACATGACGGAGGTTGTGAGGCGCTGCCCCCA |
| | CCATGAGCGCTGCTCAGATAGCGATG |
| Exon 5 amino acid sequence^{c} | YSPALNKMFCQLAKTCPVQLWVDSTPPPGTRVRAMAIYKQ |
| (SEQ ID NO: 44) | SQHMTEVVRRCPHHERCSDSD |
| Exon 6 nucleotide sequence | GTCTGGCCCCTCCTCAGCATCTTATCCGAGTGGAAGGAAA |
| (SEQ ID NO: 45) | TTTGCGTGTGGAGTATTTGGATGACAGAAACACTTTTCGA |
| | CATAGTGTGGTGGTGCCCTATGAGCCGCCTGAG |
| Exon 6 amino acid sequence^{d} | GLAPPQHLIRVEGNLRVEYLDDRNTFRHSVVVPYEPPE |
| (SEQ ID NO: 46) | |
| Exon 7 nucleotide sequence | GTTGGCTCTGACTGTACCACCATCCACTACAACTACATGT |
| (SEQ ID NO: 47) | GTAACAGTTCCTGCATGGGCGGCATGAACCGGAGGCCCA |
| | TCCTCACCATCATCACACTGGAAGACTCCAG |
| Exon 7 amino acid sequence^{e} | VGSDCTTIHYNYMCNSSCMGGMNRRPILTIITLEDS |
| (SEQ ID NO: 48) | |
| Exon 8 nucleotide sequence | TGGTAATCTACTGGGACGGAACAGCTTTGAGGTGCGTGTT |
| (SEQ ID NO: 49) | TGTGCCTGTCCTGGGAGAGACCGGCGCACAGAGGAAGAG |
| | AATCTCCGCAAGAAAGGGGAGCCTCACCACGAGCTGCCC |
| | CCAGGGAGCACTAAGCGAG |
| Exon 8 amino acid sequence^{f} | SGNLLGRNSFEVRVCACPGRDRRTEEENLRKKGEPHHELPP |
| (SEQ ID NO: 50) | GSTKR |
| Exon 9 nucleotide sequence | CACTGCCCAACAACACCAGCTCCTCTCCCCAGCCAAAGA |
| (SEQ ID NO: 51) | AGAAACCACTGGATGGAGAATATTTCACCCTTCAG |
| Exon 9 amino acid sequence^{g} | ALPNNTSSSPQPKKKPLDGEYFTLQ |
| (SEQ ID NO: 52) | |
| Exon 10 nucleotide sequence | ATCCGTGGGCGTGAGCGCTTCGAGATGTTCCGAGAGCTG |
| (SEQ ID NO: 53) | AATGAGGCCTTGGAACTCAAGGATGCCCAGGCTGGGAAG |
| | GAGCCAGGGGGGAGCAGGGCTCACTCCAG |
| Exon 10 amino acid sequence^{h} | IRGRERFEMFRELNEALELKDAQAGKEPGGSRAHS |
| (SEQ ID NO: 54) | |
| Exon 11 nucleotide sequence | CCACCTGAAGTCCAAAAAGGGTCAGTCTACCTCCCGCCAT |
| (SEQ ID NO: 55) | AAAAAACTCATGTTCAAGACAGAAGGGCCTGACTCAGAC |
| | TGACATTCTCCACTTCTTGTTCCCCACTGACAGCCTCCCAC |
| | CCCCATCTCTCCCTCCCCTGCCATTTTGGGTTTTGGGTCTT |
| | TGAACCCTTGCTTGCAATAGGTGTGCGTCAGAAGCACCCA |
| | GGACTTCCATTTGCTTTGTCCCGGGGCTCCACTGAACAAG |
| | TTGGCCTGCACTGGTGTTTTGTTGTGGGGAGGAGGATGGG |
| | GAGTAGGACATACCAGCTTAGATTTTAAGGTTTTTACTGT |
| | GAGGGATGTTTGGGAGATGTAAGAAATGTTCTTGCAGTT |
| | AAGGGTTAGTTTACAATCAGCCACATTCTAGGTAGGGGC |
| | CCACTTCACCGTACTAACCAGGGAAGCTGTCCCTCACTGT |
| | TGAATTTTCTCTAACTTCAAGGCCCATATCTGTGAAATGC |
| | TGGCATTTGCACCTACCTCACAGAGTGCATTGTGAGGGTT |
| | AATGAAATAATGTACATCTGGCCTTGAAACCACCTTTTAT |
| | TACATGGGGTCTAGAACTTGACCCCCTTGAGGGTGCTTGT |
| | TCCCTCTCCCTGTTGGTCGGTGGGTTGGTAGTTTCTACAGT |
| | TGGGCAGCTGGTTAGGTAGAGGGAGTTGTCAAGTCTCTG |
| | CTGGCCCAGCCAAACCCTGTCTGACAACCTCTTGGTGAAC |
| | CTTAGTACCTAAAAGGAAATCTCACCCCATCCCACACCCT |
| | GGAGGATTTCATCTCTTGTATATGATGATCTGGATCCACC |
| | AAGACTTGTTTTATGCTCAGGGTCAATTTCTTTTTTCTTTT |
| | TTTTTTTTTTTTTTCTTTTTCTTTGAGACTGGGTCTCGCTTT |
| | GTTGCCCAGGCTGGAGTGGAGTGGCGTGATCTTGGCTTAC |
| | TGCAGCCTTTGCCTCCCCGGCTCGAGCAGTCCTGCCTCAG |
| | CCTCCGGAGTAGCTGGGACCACAGGTTCATGCCACCATG |
| | GCCAGCCAACTTTTGCATGTTTTGTAGAGATGGGGTCTCA |
| | CAGTGTTGCCCAGGCTGGTCTCAAACTCCTGGGCTCAGGC |
| | GATCCACCTGTCTCAGCCTCCCAGAGTGCTGGGATTACAA |
| | TTGTGAGCCACCACGTCCAGCTGGAAGGGTCAACATCTTT |
| | TACATTCTGCAAGCACATCTGCATTTTCACCCCACCCTTC |
| | CCCTCCTTCTCCCTTTTTATATCCCATTTTTATATCGATCTC |
| | TTATTTTACAATAAAACTTTGCTGCCA |
| Exon 11 amino acid sequenceⁱ | SHLKSKKGQSTSRHKKLMFKTEGPDSD |
| (SEQ ID NO: 56) | |

^{a}28 nucleotides of this exon are non-coding; last 2 nucleotides of this exon are for the first amino acid in Exon 3
^{b}The first amino acid of this exon utilizes the last 2 nucleotides of Exon 2
^{c}The last nucleotide of this exon is for the first amino acid in Exon 6
^{d}The first amino acid of this exon utilizes the last nucleotide of Exon 5
^{e}The last 2 nucleotides of this exon are for the first amino acid in Exon 8
^{f}The first amino acid of this exon utilizes the last 2 nucleotides of Exon 7, and the last nucleotide of this exon is for the first amino acid in Exon 9
^{g}The first amino acid of this exon utilizes the last nucleotide of Exon 8
^{h}The last 2 nucleotides of this exon are for the first amino acid in Exon 11
ⁱThe first amino acid of this exon utilizes the last 2 nucleotides of Exon 10

### EMBODIMENTS

The following non-limiting embodiments provide illustrative examples of the invention but do not limit the scope of the invention.

Embodiment 1. An engineered non-human mammalian cell comprising a nucleic acid that encodes a p53 protein, wherein the nucleic acid comprises a sequence with at least 90% sequence identity to exon 6 of human TP53, wherein the sequence with at least 90% sequence identity to exon 6 of human TP53 encodes an amino acid substitution at position 220 relative to human p53.

Embodiment 2. The engineered non-human mammalian cell of embodiment 1, wherein the amino acid substitution is a Y220C substitution.

Embodiment 3. The engineered non-human mammalian cell of embodiment 1, wherein the amino acid substitution is a Y220S substitution.

Embodiment 4. The engineered non-human mammalian cell of embodiment 1, wherein the amino acid substitution is a Y220H substitution.

Embodiment 5. The engineered non-human mammalian cell of any one of embodiments 1-4, wherein the nucleic acid further comprises a sequence with at least 90% sequence identity to exon 5 of human TP53 and a sequence with at least 90% sequence identity to exon 7 of human TP53.

Embodiment 6. The engineered non-human mammalian cell of any one of embodiments 1-5, wherein the nucleic acid further comprises a sequence with at least 90% sequence identity to exon 4 of human TP53, a sequence with at least 90% sequence identity to exon 8 of human TP53, and a sequence with at least 90% sequence identity to exon 9 of human TP53.

Embodiment 7. The engineered non-human mammalian cell of any one of embodiments 1-6, wherein the human TP53 comprises the amino acid sequence of SEQ ID NO: 1 or SEQ ID NO: 10.

Embodiment 8. The engineered non-human mammalian cell of any one of embodiments 1-6, wherein the human TP53 comprises the amino acid sequence of any one of SEQ ID NOs: 2-9.

Embodiment 9. The engineered non-human mammalian cell of any one of embodiments 1-8, wherein the nucleic acid that encodes the p53 protein comprises an exon from a non-human TP53 gene.

Embodiment 10. The engineered non-human mammalian cell of any one of embodiments 1-9, wherein the nucleic acid that encodes the p53 protein further comprises an exon from a mouse TP53 gene.

Embodiment 11. The engineered non-human mammalian cell of any one of embodiments 1-10, wherein the nucleic acid that encodes the p53 protein further comprises exons 1-2 of a mouse TP53 gene.

Embodiment 12. The engineered non-human mammalian cell of any one of embodiments 1-11, wherein the nucleic acid that encodes the p53 protein further comprises exons 10-11 of a mouse TP53 gene.

Embodiment 13. The engineered non-human mammalian cell of any one of embodiments 1-12, wherein the nucleic acid that encodes the p53 protein is integrated into a genome of the non-human mammalian cell.

Embodiment 14. The engineered non-human mammalian cell of any one of embodiments 1-13, wherein the p53 protein is constitutively expressed from an endogenous p53 promoter of the non-human mammalian cell.

Embodiment 15. The engineered non-human mammalian cell of any one of embodiments 1-13, wherein the p53 protein is constitutively expressed by the non-human mammalian cell.

Embodiment 16. The engineered non-human mammalian cell of any one of embodiments 1-13, wherein expression of the p53 protein by the non-human mammalian cell is inducible.

Embodiment 17. An assay comprising: (a) contacting a population of engineered non-human mammalian cells with a therapeutic agent, wherein the engineered non-human mammalian cells each comprise a nucleic acid that encodes a p53 protein, wherein the nucleic acid comprises a sequence with at least 90% sequence identity to exon 6 of human TP53, wherein the sequence with at least 90% sequence identity to exon 6 of human TP53 encodes an amino acid substitution at position 220 relative to human p53; and (b) after the contacting, observing an effect of the therapeutic agent on the population of engineered non-human mammalian cells.

Embodiment 18. The assay of embodiment 17, wherein the observing the effect of the therapeutic agent comprises observing a viability of the population of engineered non-human mammalian cells in response to the therapeutic agent.

Embodiment 19. The assay of embodiment 17 or 18, wherein the observing the effect of the therapeutic agent comprises observing a metabolic activity of the population of engineered non-human mammalian cells in response to the therapeutic agent.

Embodiment 20. The assay of any one of embodiments 17-19, wherein the observing the effect of the therapeutic agent comprises observing a conformation of the p53 protein in the population of engineered non-human mammalian cells.

Embodiment 21. The assay of any one of embodiments 17-20, wherein the observing the effect of the therapeutic agent comprises determining an expression level of a gene in the population of engineered non-human mammalian cells.

Embodiment 22. The assay of any one of embodiments 17-21, wherein the observing the effect of the therapeutic agent comprises determining an expression level of a p53 target gene in the population of engineered non-human mammalian cells.

Embodiment 23. The assay of any one of embodiments 17-22, wherein the observing the effect of the therapeutic agent comprises determining an expression level of a protein in the population of engineered non-human mammalian cells.

Embodiment 24. The assay of any one of embodiments 17-23, wherein the candidate therapeutic agent is a p53 modulating agent.

Embodiment 25. The assay of any one of embodiments 17-23, wherein the candidate therapeutic agent is a p53 activating agent.

Embodiment 26. The assay of any one of embodiments 17-23, wherein the candidate therapeutic agent is a p53 reactivating agent.

Embodiment 27. The assay of any one of embodiments 17-23, wherein the candidate therapeutic agent is a chemotherapeutic agent.

Embodiment 28. The assay of any one of embodiments 17-23, wherein the candidate therapeutic agent is a radiotherapy.

Embodiment 29. The assay of any one of embodiments 17-23, wherein the candidate therapeutic agent is an immunotherapy.

Embodiment 30. A method of evaluating a therapeutic agent, comprising administering a therapeutically-effective amount of the therapeutic agent to a subject with a cancer, wherein the cancer comprises an engineered non-human mammalian cell, wherein the engineered non-human mammalian cell comprises a nucleic acid that encodes a p53 protein, wherein the nucleic acid comprises a sequence with at least 90% sequence identity to exon 6 of human TP53, wherein the sequence with at least 90% sequence identity to exon 6 of human TP53 encodes an amino acid substitution at position 220 relative to human p53.

Embodiment 31. The method of embodiment 30, wherein the cancer is a sarcoma.

Embodiment 32. The method of embodiment 30, wherein the cancer is a carcinoma.

Embodiment 33. The method of embodiment 30, wherein the cancer is a leukemia.

Embodiment 34. The method of embodiment 30, wherein the cancer is a lymphoma.

Embodiment 35. The method of embodiment 30, wherein the cancer is a myeloma.

Embodiment 36. The method of any one of embodiments 30-35, wherein the subject is a mouse.

Embodiment 37. The method of any one of embodiments 30-36, wherein the subject is syngeneic to the engineered non-human mammalian cell.

Embodiment 38. The method of any one of embodiments 30-37, wherein the candidate therapeutic agent is a p53 modulating agent.

Embodiment 39. The method of any one of embodiments 30-37, wherein the candidate therapeutic agent is a p53 activating agent.

Embodiment 40. The method of any one of embodiments 30-37, wherein the candidate therapeutic agent is a p53 reactivating agent.

Embodiment 41. The method of any one of embodiments 30-37, wherein the candidate therapeutic agent is a chemotherapeutic agent.

Embodiment 42. The method of any one of embodiments 30-37, wherein the candidate therapeutic agent is a radiotherapy.

Embodiment 43. The method of any one of embodiments 30-37, wherein the candidate therapeutic agent is an immunotherapy.

Embodiment 44. The method of any one of embodiments 30-43, further comprising administering a therapeutically-effective amount of an additional therapeutic agent to the subject.

Embodiment 45. The method of embodiment 44, wherein the additional therapeutic agent is an immunomodulator.

Embodiment 46. The method of embodiment 44, wherein the additional therapeutic agent is an immune checkpoint inhibitor.

Embodiment 47. The method of embodiment 44, wherein the additional therapeutic agent is a chemotherapeutic.

Embodiment 48. The method of embodiment 44, wherein the additional therapeutic agent is a radiotherapy.

Embodiment 49. The method of any one of embodiments 30-48, further comprising determining an effect of the candidate therapeutic agent on survival of the subject.

Embodiment 50. The method of any one of embodiments 30-49, further comprising determining an effect of the candidate therapeutic agent on tumor volume in the subject.

Embodiment 51. The method of any one of embodiments 30-50, further comprising determining an effect of the candidate therapeutic agent on an anti-cancer immune response in the subject.

Embodiment 52. The method of any one of embodiments 30-51, further comprising determining a conformation of the p53 protein in the subject in response to the therapeutic agent.

Embodiment 53. The method of any one of embodiments 30-52, further comprising determining an expression level of a gene in the subject in response to the therapeutic agent.

Embodiment 54. The method of any one of embodiments 30-53, further comprising determining an expression level of a p53 target gene in the subject in response to the therapeutic agent.

Embodiment 55. The method of any one of embodiments 30-54, further comprising determining an expression level of a protein in the subject in response to the therapeutic agent.

Embodiment 56. The method of any one of embodiments 30-55, further comprising evaluating a pharmacokinetic parameter of the therapeutic agent in the subject in response to the therapeutic agent.

Embodiment 57. A non-human animal comprising a nucleic acid that encodes a p53 protein, wherein the nucleic acid comprises a sequence with at least 90% sequence identity to exon 6 of human TP53, wherein the sequence with at least 90% sequence identity to exon 6 of human TP53 encodes an amino acid substitution at position 220 relative to human p53.

Embodiment 58. The non-human animal of embodiment 57, wherein the nucleic acid that encodes the p53 protein is integrated into a genome of the non-human animal.

Embodiment 59. The non-human animal of embodiment 57 or embodiment 58, wherein the amino acid substitution is a Y220C substitution.

Embodiment 60. The non-human animal of embodiment 57 or embodiment 58, wherein the amino acid substitution is a Y220S substitution.

Embodiment 61. The non-human animal of embodiment 57 or embodiment 58, wherein the amino acid substitution is a Y220H substitution.

Embodiment 62. The non-human animal of any one of embodiments 57-61, wherein the non-human animal is homozygous for the nucleic acid that encodes the p53 protein.

Embodiment 63. The non-human animal of any one of embodiments 57-62, wherein the non-human animal is heterozygous for the nucleic acid that encodes the p53 protein.

Embodiment 64. The non-human animal of any one of embodiments 57-63, wherein expression of the p53 protein is from an endogenous p53 promoter of the non-human animal.

Embodiment 65. The non-human animal of any one of embodiments 57-63, wherein expression of the p53 protein is tissue-specific.

Embodiment 66. The non-human animal of any one of embodiments 57-63, wherein expression of the p53 protein is cell type-specific.

Embodiment 67. The non-human animal of any one of embodiments 57-63, wherein expression of the p53 protein is constitutive.

Embodiment 68. The non-human animal of any one of embodiments 57-63, wherein expression of the p53 protein is inducible.

Embodiment 69. The non-human animal of any one of embodiments 57-68, wherein the non-human animal is a mouse.

Embodiment 70. The non-human animal of any one of embodiments 57-69, wherein the non-human animal is immunocompetent.

Embodiment 71. The non-human animal of any one of embodiments 57-69, wherein the non-human animal is immunodeficient.

Embodiment 72. The non-human animal of any one of embodiments 57-69, wherein the non-human animal is an immunocompetent mouse.

Embodiment 73. The non-human animal of any one of embodiments 57-72, wherein the non-human animal is a mouse with a C57BL/6 genetic background.

Embodiment 74. The non-human animal of any one of embodiments 57-72, wherein the non-human animal is a mouse with a BALB/C, C3H, DBA/2J, A/J, or FVB/N genetic background, or a mixed genetic background.

Embodiment 75. A non-human animal comprising the engineered non-human mammalian cell of any of embodiments 1-16.

1. An engineered non-human mammalian cell comprising a nucleic acid that encodes a p53 protein, wherein the nucleic acid comprises a sequence with at least 90% sequence identity to exon 6 of human TP53, wherein the sequence with at least 90% sequence identity to exon 6 of human TP53 encodes an amino acid substitution at position 220 relative to human p53.

2. The engineered non-human mammalian cell of claim 1, wherein the amino acid substitution is a Y220C substitution.

3. The engineered non-human mammalian cell of claim 1, wherein the amino acid substitution is a Y220S substitution.

4. The engineered non-human mammalian cell of claim 1, wherein the amino acid substitution is a Y220H substitution.

5. The engineered non-human mammalian cell of claim 1, wherein the nucleic acid further comprises a sequence with at least 90% sequence identity to exon 5 of human TP53 and a sequence with at least 90% sequence identity to exon 7 of human TP53.

6. The engineered non-human mammalian cell of claim 5, wherein the nucleic acid further comprises a sequence with at least 90% sequence identity to exon 4 of human TP53, a sequence with at least 90% sequence identity to exon 8 of human TP53, and a sequence with at least 90% sequence identity to exon 9 of human TP53.

7. The engineered non-human mammalian cell of claim 1, wherein the human TP53 comprises the amino acid sequence of SEQ ID NO: 1 or SEQ ID NO: 10.

8. The engineered non-human mammalian cell of claim 1, wherein the human TP53 comprises the amino acid sequence of any one of SEQ ID NOs: 2-9.

9. The engineered non-human mammalian cell of claim 1, wherein the nucleic acid that encodes the p53 protein comprises an exon from a non-human TP53 gene.

10. The engineered non-human mammalian cell of claim 1, wherein the nucleic acid that encodes the p53 protein further comprises an exon from a mouse TP53 gene.

11. The engineered non-human mammalian cell of claim 1, wherein the nucleic acid that encodes the p53 protein further comprises exons 1-2 of a mouse TP53 gene.

12. The engineered non-human mammalian cell of claim 1, wherein the nucleic acid that encodes the p53 protein further comprises exons 10-11 of a mouse TP53 gene.

13. The engineered non-human mammalian cell of claim 1, wherein the nucleic acid that encodes the p53 protein is integrated into a genome of the non-human mammalian cell.

14. The engineered non-human mammalian cell of claim 1, wherein the p53 protein is constitutively expressed from an endogenous p53 promoter of the non-human mammalian cell.

15. The engineered non-human mammalian cell of claim 1, wherein the p53 protein is constitutively expressed by the non-human mammalian cell.

16. The engineered non-human mammalian cell of claim 1, wherein expression of the p53 protein by the non-human mammalian cell is inducible.

17-74. (canceled)

75. A non-human animal comprising an engineered non-human mammalian cell, wherein the engineered non-human mammalian cell comprises a nucleic acid that encodes a p53 protein, wherein the nucleic acid comprises a sequence with at least 90% sequence identity to exon 6 of human TP53, wherein the sequence with at least 90% sequence identity to exon 6 of human TP53 encodes an amino acid substitution at position 220 relative to human p53.

76. The non-human animal of claim 75, wherein the amino acid substitution is a Y220C substitution.

77. The non-human animal of claim 75, wherein the nucleic acid that encodes the p53 protein further comprises an exon from a mouse TP53 gene.

78. The non-human animal of claim 75, wherein the nucleic acid that encodes the p53 protein is integrated into a genome of the non-human mammalian cell.
